SEQUENCE LISTING (96 sequences)

SEQ ID NO: 1 - DNA, 408 bp, Artificial sequence
  Synthetic oligonucleotide
caggtgcagctggtgcagtctggggctgaggtgaagaagcctggggcctcactgaaggtctcctacaaggcctctggatacaccttcatcgcctactatgcgcactgggtgcgacaggcccctggacaagggcttgagtggatgggacggatcaacccgaacagtggtggcacaaagtatcacagaggtttcagggcagggtcaccgtgaccagggacacgtccttcaccacagcctgcctggaaatgaacaggctaacatctgacgacacggccgtatttacttgtgcgagtaaatattgcgctattgtagtaggagcagctgccgtactcgagatagcaacagccaagaccgtccccctcaagatcggatgatggggccagggaaccctggtcagaagggatttgg

SEQ ID NO: 2 - DNA, 331 bp, Artificial sequence
  Synthetic oligonucleotide
cagtctgtggtgactcaaccaccctcgacgtctgggacccccgggcagagggtcaccatctcttgttctggaagcagcgccaacatcggaagaaatgttgtgaactggtaccagcaggtcccaggaacggcccccaaactcctcatctttggtaatagtcagcggccctcaagggtccctgaccgattctctggctccaagtctggcacctcagcctccctggccatcagtgggctccagtctgaggatgaggctgattattattgtgcaacgtgggatgacagcctgaatggtccggtcttcggcggagggacccaggtgaccgtcctag

SEQ ID NO: 3 - DNA, 361 bp, Artificial sequence
  Synthetic oligonucleotide
caggtgcagctggtgcagtctgggggaggcgtggtccagcctgggaggtccctgagactctcctgtgcagcctctggattccccttcagaatctactctatgcactgggtccgccaggctccaggcaaggggctggagtgggtggcactcatctcatatgatggaaccaataaacagtacgcagactccgtgaacggccgattcaccatctccagagacaattccgagaacacgatgtatttgcaaatgaacagtctgagacctgaggacacggctatctattactgcgcgacagatattgtcgaactggtgactgctactgactactggggccagggaaccctggtcaccgtctcctcag

SEQ ID NO: 4 - DNA, 322 bp, Artificial sequence
  Synthetic oligonucleotide
aggctgtggtgactcagtctccaggcaccctgtctttgtctccaggggacaggagccaccctctcctgcagggccagtcagagtgttagcacctccttaggctggtaccagcagaaacctggccagtcgcccaggctcctcatctatgggacatccagaagggccactggcgtcccggacaggttcagtggcagtggatctgagacagacttcactctcaccatcagcagactggagcctgaagatttttcagtgtattactgtcagcagtatggtagttcaccttacacttttggccaggggaccaggctggagatcaaac

SEQ ID NO: 5 - DNA, 378 bp, Artificial sequence
  Synthetic oligonucleotide
caggtccagctgggggagtctggtcctgcgctggtgaaacccacacagaccctcacactgacctgcacgttctctgggttctcactcagcacgagtgaaatgtgtgtgagctggatccgtcagcccccagggaaggccctggagtggcttgcactcattgattgggatggtgataaattcttcagtacatctctgcagtccaggctcaccatctccaagagcccctccaataaccaggtggtccttacaatgaccaacatggaccctgtggactcaggcacctatttctgtgcacggtctactgttcgcaggtcgtccggctactactactatgttttggacgtctggggccaaggaaccctggtcaccgtctcctca

SEQ ID NO: 6 - DNA, 319 bp, Artificial sequence
  Synthetic oligonucleotide
cagattgtgatgactcagtctccatcctccctgtccgcctctgtcggagacagagtcaccatcagttgtcgggcaagtcagagcatcggcacctatgtaaattggtatcaacacaagccagggaaagcccctaaggtcctgatctctggtgcctccaatttgcacagtggggtcccatccaggttcagtggcagtggatctgggacagacttcactctcaccatcagcagtctgcaacctgaagattttgcaacttactactgtcaacagagttacagtccgctcactttcggcggagggaccacggtggagatgaaag

SEQ ID NO: 7 - DNA, 376 bp, Artificial sequence
  Synthetic oligonucleotide
caggtgcagctggtgcagtctgggggaggcctggcacagccagggcggtccctgagactctcctgtagagcttctgggttcacctttggtgattttaatatgaactggttccgccaggctccagggaaggggctggagtgggtaggattcattagaaggaaagcttttggtggggcaacagaatacgccgcgtcggtgaaaggcagactcaccatctcaagggatgattccaagagcatcgcctatctgcaaatgaacagcctgaaaaccgaggacacagccgtgtattactgtactagagaacggggatatgttggttcgggggggcccttctttgactactggggccagggaaccctggtcaccgtctcctcag

SEQ ID NO: 8 - DNA, 334 bp, Artificial sequence
  Synthetic oligonucleotide
caggctgtggtgactcagccgccctcagtgtctggggccccagggcagagggtcaccatctcctgcactgggagcagctccaacatcggggcaggttatgatgtacactggtaccagcaacttccaggaacagcccccaaactcctcatctatggtgacagcaatcggccctcaggggtccctgaccgattctctggctccagctctggcacctcagcctccctggccatcactgggctccaggctgaggatgaggctgattattactgccagtcctatgacaacagcctgagtggttctgtcttcggaactgggaccaaggtcaccgtcctag

SEQ ID NO: 9 - DNA, 373 bp, Artificial sequence
  Synthetic oligonucleotide
caggtgcagctggtgcagtctggggctgaggtgaagaagcctgggtcctcagtgacggtctcctgcaaggcttctggaggcagcttcaccaactatgctttcagctgggtgcgacaggcccctggacaagggcttgagtggatgggcgggatcatccctctccttaatatgccaaattacgcacagaagtttcggggcagagtcacgatttccgcggaccaatccaccaccacagcctacatggaactgagcagactgacatctgaagacacggccatctatttctgtgcgagagggggtcaagttggagattttatcgttcttcgtcactttgactcctggggccaaggaaccctggtcaccgtctcctcag

SEQ ID NO: 10 - DNA, 270 bp, Artificial sequence
  Synthetic oligonucleotide
ccaccctctcctgcagggccagtgagagtgttagcaactacttagcctggtatcagcagaaacctgggcagactcccagactcctcatctatggtgcatccacgagggccactggtatcccagccaggttcagtggcagtgggtctgggtcagagttcactctcaccatcagcagcctgcagtctgaagattttgcggtttattattgtcagcagtataatgactggcccaggttcagttttggccaggggaccaagctggagatcaaac

SEQ ID NO: 11 - DNA, 367 bp, Artificial sequence
  Synthetic oligonucleotide
caggtgcagctggtgcagtctgggggaggcgtggtccagcctgggcagtccctgagactctcctgtgcagcctctggattcagtttcagtgactatcctatacactgggtccgccaggctccaggcaaggggctggaatgggtggcaggaatttcatattatggatccaataaattttacgcagactccgtgaggggccgcttcaccatctcccgagacacttccaagaacacatttaatctgcaaatgaacagcctgaaaagtgaggacacggctgtgtattactgtgcgagagatggcaaccccccccgatttttggaatactttgactactggggccagggaaccctggtcaccgtctcctcag

SEQ ID NO: 12 - DNA, 334 bp, Artificial sequence
  Synthetic oligonucleotide
cagcctgtggtgactcagcctcgctcagtgtccgggtctcctggacagtcagtcaccatctcctgcactgggagcagcagtgatgtcggtggttataactttgtctcctggtaccgacatcacccaggcaaggcccccaaactcctcatttatcatgtcactaagcggccctcaggggtccctgatcgcttctctggctccaagtctggcaacacggcctccctgaccatctctgggctccaggctgaggatgaggctgattattactgctgctcatatgcaggcagctatacttatgttctattcggcggagggaccaagctgaccgtcctag

SEQ ID NO: 13 - DNA, 364 bp, Artificial sequence
  Synthetic oligonucleotide
caggtgcagctggtggagtctggaggaggcttgatccagcctggggggtccctgagactctcctgtgcagtctcggggttcaccgtcagtagcaagtacatgacctgggtccgccaggctccagggaaggggctggaatgggtctcagttatttatggcggtggtagcacatactacgcagactccgtggtgggccgattcaccatctccagagacaattccaagaacacgttgtatcttcaaatgaacagcctgagagccgaggacacggccgtgtattactgtgcgagtcgattaggggttcgggcaactacgggcgatcttgactactggggccagggaaccctggtcaccgtctcctcag

SEQ ID NO: 14 - DNA, 325 bp, Artificial sequence
  Synthetic oligonucleotide
cagattgtgatgacccagtctccttccaccctgtctgcatctgtaggagacagagtcaccatcacttgccgggccagtcagagtattagtagctggttggcctggtatcagcagaaaccagggaaagcccctaaactcctgatctatgatgcctccagtttggaaagtggggtcccatcaaggttcagcggcagtggatctgggacagaattcactctcaccatcagcagcctgcagcctgatgattttgcaacttattactgccaacagtataatacttattcttggtggacgttcggccaagggaccaaggtggaaatcaaac

SEQ ID NO: 15 - DNA, 361 bp, Artificial sequence
  Synthetic oligonucleotide
caggtgcagctggtggagtctgggggaggcgtggtccagcctgggaagtccctgagactctcctgtgcagcctctggatacatcttcagtagctatgacatgcactgggtccgccaggctccaggcaaggggctggagtgggtggcagttatttcatttgacggaactactcaacactatgcagactctgtgaggggccgattcaccgtctccagagacaattcccagaacacggtgtttctgcaaatgaacagcctgagacctgaggacacggctgtgtattactgtgtgaaggaatatgtgattgtgtcgactttctttgactactggggccagggaaccctggtcaccgtctcctcag

SEQ ID NO: 16 - DNA, 322 bp, Artificial sequence
  Synthetic oligonucleotide
gacatcgtgatgacccagtctccatcctccctgtctgcatctgtaggagacagagtcaccatcacttgccgggcaagtcagggcattagaaatgctttaggctggtatcagcacaaaccagggaaagcccctaaggtcctgatctatgctgcatcccgtttacaaagtggggtcccatcaaggttcagcggcagtggatctggcacagatttcactctcaccatcagcagcctgcagcctgaagattttgcaacttattactgtcttcaagatttcaattacccgtggacgttcggccacgggaccaaggtggaaatcaaac

SEQ ID NO: 17 - DNA, 376 bp, Artificial sequence
  Synthetic oligonucleotide
gaggtgcagctggtggagtctgggggaggcttggtacagcctggggggtccctgagactctcctgtgcggcctctggattcacctttagcagttttaccatgaactgggtccgccaggctccagggaaggggctgcagtgggtctcaactattagtggtagtggtggtctcacatactacgcagactccgtgaagggccggttcaccatctccagagacaattccaagaacacgctgtctctgcaaatgaacagcctgagagccgaggacacggccgtatattactgtgcgagagatctcgaatttacggtgacttcctacgggggatactactttgagtactggggccagggaaccctggtcaccgtctcctcag

SEQ ID NO: 18 - DNA, 328 bp, Artificial sequence
  Synthetic oligonucleotide
gaaattgtgttgactcagtctccaggcaccctgtctttgtctccaggggaaagagccaccctctcctgcagggccagtcagagtgttagcagcaactacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtttggtagctcaccccgattcactttcggccctgggaccaaagtggatatcaaac

SEQ ID NO: 19 - DNA, 21 bp, Artificial sequence
  Synthetic oligonucleotide
  modified_base (1)..(20): phosphorothioate-modified oligodeoxynucleotide
tcgtcgtttttcggtcgtttt

SEQ ID NO: 20 - protein, 136 aa, Artificial sequence
  Synthetic amino acid sequence
QVQLVQSGAEVKKPGASLKVSYKASGYTFIAYYAHWVRQAPGQGLEWMGRINPNSGGTKYTQRFQGRVTVTRDTSFTTACLEMNRLTSDDTAVFTCASKYCAIVVGAAAVLEIATAKTVPLKIGNWGQGTLVRRDL

SEQ ID NO: 21 - protein, 110 aa, Artificial sequence
  Synthetic amino acid sequence
QSVVTQPPSTSGTPGQRVTISCSGSSANIGRNVVNWYQQVPGTAPKLLIFGNSQRPSRVPDRFSGSKSGTSASLAISGLQSEDEADYYCATWDDSLNGPVFGGGTQVTVL

SEQ ID NO: 22 - protein, 120 aa, Artificial sequence
  Synthetic amino acid sequence
QVQLVQSGGGVVQPGRSLRLSCAASGFPFRIYSMHWVRQAPGKGLEWVALISYDGTNKQYADSVNGRFTISRDNSENTMYLQMNSLRPEDTAIYYCATDIVELVTATDYWGQGTLVTVSS

SEQ ID NO: 23 - protein, 103 aa, Artificial sequence
  Synthetic amino acid sequence
LSLQAPCLCLQGTGATLSCRASQSVSTSLGWYQQKPGQSPRLLIYGTSRRATGVPDRFSGSGSETDFTLTISRLEPEDFSVYYCQQYGSSPYTFGQGTRLEIK

SEQ ID NO: 24 - protein, 126 aa, Artificial sequence
  Synthetic amino acid sequence
QVQLGESGPALVKPTQTLTLTCTFSGFSLSTSEMCVSWIRQPPGKALEWLALIDWDGDKFFSTSLQSRLTISKSPSNNQVVLTMTNMDPVDSGTYFCARSTVRRSSGYYYYVLDVWGQGTLVTVSS

SEQ ID NO: 25 - protein, 106 aa, Artificial sequence
  Synthetic amino acid sequence
QIVMTQSPSSLSASVGDRVTISCRASQSIGTYVNWYQHKPGKAPKVLISGASNLHSGVPSRFSGSGSGTDFTLTISSLQPEDFATYYCQQSYSPLTFGGGTTVEMK

SEQ ID NO: 26 - protein, 125 aa, Artificial sequence
  Synthetic amino acid sequence
QVQLVQSGGGLAQPGRSLRLSCRASGFTFGDFNMNWFRQAPGKGLEWVGFIRRKAFGGATEYAASVKGRLTISRDDSKSIAYLQMNSLKTEDTAVYYCTRERGYVGSGGPFFDYWGQGTLVTVSS

SEQ ID NO: 27 - protein, 111 aa, Artificial sequence
  Synthetic amino acid sequence
QAVVTQPPSVSGAPGQRVTISCTGSSSNIGAGYDVHWYQQLPGTAPKLLIYGDSNRPSGVPDRFSGSSSGTSASLAITGLQAEDEADYYCQSYDNSLSGSVFGTGTKVTVL

SEQ ID NO: 28 - protein, 124 aa, Artificial sequence
  Synthetic amino acid sequence
QVQLVQSGAEVKKPGSSVTVSCKASGGSFTNYAFSWVRQAPGQGLEWMGGIIPLLNMPNYAQKFRGRVTISADQSTTTAYMELSRLTSEDTAIYFCARGGQVGDFIVLRHFDSWGQGTLVTVSS

SEQ ID NO: 29 - protein, 89 aa, Artificial sequence
  Synthetic amino acid sequence
TLSCRASESVSNYLAWYQQKPGQTPRLLIYGASTRATGIPARFSGSGSGSEFTLTISSLQSEDFAVYYCQQYNDWPRFSFGQGTKLEIK

SEQ ID NO: 30 - protein, 122 aa, Artificial sequence
  Synthetic amino acid sequence
QVQLVQSGGGVVQPGQSLRLSCAASGFSFSDYPIHWVRQAPGKGLEWVAGISYYGSNKFYADSVRGRFTISRDTSKNTFNLQMNSLKSEDTAVYYCARDGNPPRFLEYFDYWGQGTLVTVSS

SEQ ID NO: 31 - protein, 111 aa, Artificial sequence
  Synthetic amino acid sequence
QPVVTQPRSVSGSPGQSVTISCTGSSSDVGGYNFVSWYRHHPGKAPKLLIYHVTKRPSGVPDRFSGSKSGNTASLTISGLQAEDEADYYCCSYAGSYTYVLFGGGTKLTVL

SEQ ID NO: 32 - protein, 121 aa, Artificial sequence
  Synthetic amino acid sequence
QVQLVESGGGLIQPGGSLRLSCAVSGFTVSSKYMTWVRQAPGKGLEWVSVIYGGGSTYYADSVVGRFTISRDNSKNTLYLQMNSLRAEDTAVYYCASRLGVRATTGDLDYWGQGTLVTVSS

SEQ ID NO: 33 - protein, 108 aa, Artificial sequence
  Synthetic amino acid sequence
QIVMTQSPSTLSASVGDRVTITCRASQSISSWLAWYQQKPGKAPKLLIYDASSLESGVPSRFSGSGSGTEFTLTISSLQPDDFATYYCQQYNTYSWWTFGQGTKVEIK

SEQ ID NO: 34 - protein, 120 aa, Artificial sequence
  Synthetic amino acid sequence
QVQLVESGGGVVQPGKSLRLSCAASGYIFSSYDMHWVRQAPGKGLEWVAVISFDGTTQHYADSVRGRFTVSRDNSQNTVFLQMNSLRPEDTAVYYCVKEYVIVSTFFDYWGQGTLVTVSS

SEQ ID NO: 35 - protein, 107 aa, Artificial sequence
  Synthetic amino acid sequence
DIVMTQSPSSLSASVGDRVTITCRASQGIRNALGWYQHKPGKAPKVLIYAASRLQSGVPSRFSGSGSGTDFTLTISSLQPEDFATYYCLQDFNYPWTFGHGTKVEIK

SEQ ID NO: 36 - protein, 125 aa, Artificial sequence
  Synthetic amino acid sequence
EVQLVESGGGLVQPGGSLRLSCAASGFTFSSFTMNWVRQAPGKGLQWVSTISGSGGLTYYADSVKGRFTISRDNSKNTLSLQMNSLRAEDTAVYYCARDLEFTVTSYGGYYFEYWGQGTLVTVSS

SEQ ID NO: 37 - protein, 109 aa, Artificial sequence
  Synthetic amino acid sequence
EIVLTQSPGTLSLSPGERATLSCRASQSVSSNYLAWYQQKPGQAPRLLIYGASSRATGIPDRFSGSGSGTDFTLTISRLEPEDFAVYYCQQFGSSPRFTFGPGTKVDIK

SEQ ID NO: 38 - protein, 8 aa, Artificial sequence
  Synthetic amino acid sequence
GYTFIAYY

SEQ ID NO: 39 - protein, 8 aa, Artificial sequence
  Synthetic amino acid sequence
INPNSGGT

SEQ ID NO: 40 - protein, 31 aa, Artificial sequence
  Synthetic amino acid sequence
CASKYCAIVVGAAAVLEIATAKTVPLKIGNW

SEQ ID NO: 41 - protein, 8 aa, Artificial sequence
  Synthetic amino acid sequence
GFPFRIYS

SEQ ID NO: 42 - protein, 8 aa, Artificial sequence
  Synthetic amino acid sequence
ISYDGTNK

SEQ ID NO: 43 - protein, 13 aa, Artificial sequence
  Synthetic amino acid sequence
ATDIVELVTATDY

SEQ ID NO: 44 - protein, 10 aa, Artificial sequence
  Synthetic amino acid sequence
GFSLSTSEMC

SEQ ID NO: 45 - protein, 7 aa, Artificial sequence
  Synthetic amino acid sequence
IDWDGDK

SEQ ID NO: 46 - protein, 18 aa, Artificial sequence
  Synthetic amino acid sequence
ARSTVRRSSGYYYYVLDV

SEQ ID NO: 47 - protein, 8 aa, Artificial sequence
  Synthetic amino acid sequence
GFTFGDFN

SEQ ID NO: 48 - protein, 10 aa, Artificial sequence
  Synthetic amino acid sequence
IRRKAFGGAT

SEQ ID NO: 49 - protein, 16 aa, Artificial sequence
  Synthetic amino acid sequence
TRERGYVGSGGPFFDY

SEQ ID NO: 50 - protein, 8 aa, Artificial sequence
  Synthetic amino acid sequence
GGSFTNYA

SEQ ID NO: 51 - protein, 8 aa, Artificial sequence
  Synthetic amino acid sequence
IIPLLNMP

SEQ ID NO: 52 - protein, 17 aa, Artificial sequence
  Synthetic amino acid sequence
ARGGQVGDFIVLRHFDS

SEQ ID NO: 53 - protein, 8 aa, Artificial sequence
  Synthetic amino acid sequence
GFSFSDYP

SEQ ID NO: 54 - protein, 8 aa, Artificial sequence
  Synthetic amino acid sequence
ISYYGSNK

SEQ ID NO: 55 - protein, 15 aa, Artificial sequence
  Synthetic amino acid sequence
ARDGNPPRFLEYFDY

SEQ ID NO: 56 - protein, 8 aa, Artificial sequence
  Synthetic amino acid sequence
GFTVSSKY

SEQ ID NO: 57 - protein, 7 aa, Artificial sequence
  Synthetic amino acid sequence
IYGGGST

SEQ ID NO: 58 - protein, 15 aa, Artificial sequence
  Synthetic amino acid sequence
ASRLGVRATTGDLDY

SEQ ID NO: 59 - protein, 8 aa, Artificial sequence
  Synthetic amino acid sequence
GYIFSSYD

SEQ ID NO: 60 - protein, 8 aa, Artificial sequence
  Synthetic amino acid sequence
ISFDGTTQ

SEQ ID NO: 61 - protein, 13 aa, Artificial sequence
  Synthetic amino acid sequence
VKEYVIVSTFFDY

SEQ ID NO: 62 - protein, 8 aa, Artificial sequence
  Synthetic amino acid sequence
GFTFSSFT

SEQ ID NO: 63 - protein, 8 aa, Artificial sequence
  Synthetic amino acid sequence
ISGSGGLT

SEQ ID NO: 64 - protein, 18 aa, Artificial sequence
  Synthetic amino acid sequence
ARDLEFTVTSYGGYYFEY

SEQ ID NO: 65 - protein, 8 aa, Artificial sequence
  Synthetic amino acid sequence
SANIGRNV

SEQ ID NO: 66 - protein, 3 aa, Artificial sequence
  Synthetic amino acid sequence
GNS

SEQ ID NO: 67 - protein, 11 aa, Artificial sequence
  Synthetic amino acid sequence
ATWDDSLNGPV

SEQ ID NO: 68 - protein, 6 aa, Artificial sequence
  Synthetic amino acid sequence
QSVSTS

SEQ ID NO: 69 - protein, 3 aa, Artificial sequence
  Synthetic amino acid sequence
GTS

SEQ ID NO: 70 - protein, 9 aa, Artificial sequence
  Synthetic amino acid sequence
QQYGSSPYT

SEQ ID NO: 71 - protein, 6 aa, Artificial sequence
  Synthetic amino acid sequence
QSIGTY

SEQ ID NO: 72 - protein, 3 aa, Artificial sequence
  Synthetic amino acid sequence
GAS

SEQ ID NO: 73 - protein, 8 aa, Artificial sequence
  Synthetic amino acid sequence
QQSYSPLT

SEQ ID NO: 74 - protein, 9 aa, Artificial sequence
  Synthetic amino acid sequence
SSNIGAGYD

SEQ ID NO: 75 - protein, 3 aa, Artificial sequence
  Synthetic amino acid sequence
GDS

SEQ ID NO: 76 - protein, 11 aa, Artificial sequence
  Synthetic amino acid sequence
QSYDNSLSGSV

SEQ ID NO: 77 - protein, 6 aa, Artificial sequence
  Synthetic amino acid sequence
ESVSNY

SEQ ID NO: 78 - protein, 3 aa, Artificial sequence
  Synthetic amino acid sequence
GAS

SEQ ID NO: 79 - protein, 10 aa, Artificial sequence
  Synthetic amino acid sequence
QQYNDWPRFS

SEQ ID NO: 80 - protein, 9 aa, Artificial sequence
  Synthetic amino acid sequence
SSDVGGYNF

SEQ ID NO: 81 - protein, 3 aa, Artificial sequence
  Synthetic amino acid sequence
HVT

SEQ ID NO: 82 - protein, 11 aa, Artificial sequence
  Synthetic amino acid sequence
CSYAGSYTYVL

SEQ ID NO: 83 - protein, 6 aa, Artificial sequence
  Synthetic amino acid sequence
QSISSW

SEQ ID NO: 84 - protein, 3 aa, Artificial sequence
  Synthetic amino acid sequence
DAS

SEQ ID NO: 85 - protein, 10 aa, Artificial sequence
  Synthetic amino acid sequence
QQYNTYSWWT

SEQ ID NO: 86 - protein, 6 aa, Artificial sequence
  Synthetic amino acid sequence
QGIRNA

SEQ ID NO: 87 - protein, 3 aa, Artificial sequence
  Synthetic amino acid sequence
AAS

SEQ ID NO: 88 - protein, 9 aa, Artificial sequence
  Synthetic amino acid sequence
LQDFNYPWT

SEQ ID NO: 89 - protein, 7 aa, Artificial sequence
  Synthetic amino acid sequence
QSVSSNY

SEQ ID NO: 90 - protein, 3 aa, Artificial sequence
  Synthetic amino acid sequence
GAS

SEQ ID NO: 91 - protein, 10 aa, Artificial sequence
  Synthetic amino acid sequence
QQFGSSPRFT

SEQ ID NO: 92 - protein, 234 aa, Artificial sequence
  Synthetic amino acid sequence
EVQLVESGGGLIQPGGSLRLSCAVSGFTVSSKYMTWVRQAPGKGLEWVSVIYGGGSTYYADSVVGRFTISRDNSKNTLYLQMNSLRAEDTAVYYCASRLGVRATTGDLDYWGQGTLVTVSSASTKGDIQMTQSPSTLSASVGDRVTITCRASQSISSWLAWYQQKPGKAPKLLIYDASSLESGVPSRFSGSGSGTEFTLTISSLQPDDFATYYCQQYNTYSWWTFGQGTKVDIK

SEQ ID NO: 93 - protein, 13 aa, Artificial sequence
  Synthetic amino acid sequence
LGVRATTGDLDYW

SEQ ID NO: 94 - protein, 10 aa, Artificial sequence
  Synthetic amino acid sequence
LLIYDASSLE

SEQ ID NO: 95 - protein, 17 aa, Artificial sequence
  Synthetic amino acid sequence
CASRLGVTATTGDLDYW

SEQ ID NO: 96 - protein, 12 aa, Artificial sequence
  Synthetic amino acid sequence
CQQYNTYSWWTF

(19) United States
(12) Patent Application Publication
(10) Pub. No.: US 2019/0240316 A1
(43) Pub. Date: Aug. 8, 2019

(54) HUMAN RESPIRATORY SYNCYTIAL VIRUS ANTIBODIES AND METHODS OF USE THEREFOR

(71) Applicant: Vanderbilt University, Nashville, TN (US)

(72) Inventors: James E. Crowe, Jr., Nashville, TN (US); Jarrod Mousa, Nashville, TN (US)

(73) Assignee: Vanderbilt University, Nashville, TN (US)

(21) Appl. No.: 16/342,528

(22) PCT Filed: Oct. 16, 2017
(86) PCT No.: PCT/US17/56730

§ 371 Date: Apr. 16, 2019

Related U.S. Application Data
(60) Provisional application No. 62/408,895, filed on Oct. 17, 2016.

(51) Int. Cl. A61K 39/155; C07K 14/005; G01N 33/569; C07K 16/10; A61P 31/16

(52) U.S. Cl. CPC A61K 39/155; C07K 14/005; A61P 31/16; C07K 16/1027; G01N 33/56983

(57) ABSTRACT

The present disclosure is directed to antibodies binding to human respiratory syncytial virus F protein, including both neutralizing and non-neutralizing antibodies, and methods for use thereof.

This application claims benefit of priority to U.S. Provisional Application Serial No. 62/408,895, filed Oct. 17, 2016, the entire contents of which is hereby incorporated by reference.

### BACKGROUND

### 1. Field of the Disclosure

The present disclosure relates generally to the fields of medicine, infectious disease, and immunology. More particular, the disclosure relates to human antibodies binding to respiratory syncytial virus (RSV).

### 2. Background

Respiratory syncytial virus (RSV) is a highly contagious human pathogen, infecting the majority of infants before age two, and is the leading cause of viral bronchiolitis and viral pneumonia in infants and children (Hall et al., 2009; Shefali-Patel et al., 2012). RSV remains a top priority for vaccine development, as thousands of deaths are recorded worldwide each year due to complications from infection (Nair et al., 2010). To date, there is no licensed RSV vaccine. A major focus of RSV vaccine development has been inclusion of the RSV fusion (F) protein, a class I fusion glycoprotein that is synthesized as a precursor and cleaved into two disulfide-linked fragments upon maturation into a trimer (McLellan, 2015). While the RSV virion contains two additional surface proteins, the highly-glycosylated attachment (G) protein and the small hydrophobic protein, the F protein is highly conserved among strains of RSV strains and is the major target of protective neutralizing antibodies.

The F protein is known to adopt at least two major conformations, the metastable pre-fusion conformation and the post-fusion conformation. Following attachment of the virion to a cell by the G attachment protein, the F protein undergoes a dramatic structural rearrangement resulting in fusion of the viral and cell membranes, and in cultured cells causes formation of cell syncytia. Four major neutralizing antigenic regions have been identified to date in the F protein, generally designated antigenic sites I, II, IV, and Ø, with the latter present only in the pre-fusion conformation. Site II is the target of palivizumab (Group TIm-RS, 1998), a prophylactic treatment licensed for use in high-risk infants during the RSV season. An RSV F protein subunit vaccine candidate comprising aggregates of the post-fusion conformation of RSV F is being tested currently in clinical trials (Glenn et al., 2015), and serum antibody competition with palivizumab has been proposed as a potential serologic correlate of immunity for that vaccine (Smith et al., 2012; Raghunanda et al., 2014). The inventors and others have isolated and studied RSV F-specific mAbs using murine hybridomas (Wu et al., 2007a), sorted macaque B cells (Correia et al., 2014), transformed human B cells or human antibody gene phage display libraries (Crowe et al., 1998a; 1998b). Examples include mAbs 101F (Wu et al., 2007a), D25 (McLellan et al., 2013a), and the next-generation site II mAb motavizumab (Wu et al., 2007b). However, there are no reported naturally-occurring human mAbs to site II, and palivizumab is an engineered humanized version of the murine mAb 1129 (Beeler and va Wyke Coelingh, 1989). Therefore, the repertoire of human antibodies interacting with site II and the structural basis for their recognition of this major antigenic site is poorly understood.

### SUMMARY

Thus, in accordance with the present disclosure, there is provided a method of detecting a human respiratory syncytial virus infection in a subject comprising (a) contacting a sample from said subject with an antibody or antibody fragment having clone-paired heavy and light chain CDR sequences from Tables 3 and 4, respectively; and (b) detecting human respiratory syncytial virus in said sample by binding of said antibody or antibody fragment to a Human respiratory syncytial virus antigen in said sample. The sample may be a body fluid, such as blood, sputum, tears, saliva, mucous or serum, urine, exudate, transudate, tissue scrapings or feces. Detection may comprise ELISA, RIA or Western blot. The method may further comprise performing steps (a) and (b) a second time and determining a change in human respiratory syncytial virus antigen levels as compared to the first assay.

The antibody or antibody fragment may be encoded by clone-paired variable sequences as set forth in Table 1, may be encoded by light and heavy chain variable sequences having 70%, 80%, or 90% identity to clone-paired variable sequences as set forth in Table 1, or may be encoded by light and heavy chain variable sequences having 95% identity to clone-paired sequences as set forth in Table 1. The antibody or antibody fragment may comprise light and heavy chain variable sequences according to clone-paired sequences from Table 2, may comprise light and heavy chain variable sequences having 70%, 80% or 90% identity to clone-paired sequences from Table 2 and may comprise light and heavy chain variable sequences having 95% identity to clone-paired sequences from Table 2. The antibody fragment may be a recombinant ScFv (single chain fragment variable) antibody, Fab fragment, F(ab′)₂ fragment, or Fv fragment.

Also provided is a method of treating a subject infected with human respiratory syncytial virus, or reducing the likelihood of infection of a subject at risk of contracting human respiratory syncytial virus, comprising delivering to said subject an antibody or antibody fragment having clone-paired heavy and light chain CDR sequences from Tables 3 and 4, respectively. The antibody fragment may be a recombinant ScFv (single chain fragment variable) antibody, Fab fragment, F(ab′)₂ fragment, or Fv fragment, a chimeric antibody and/or is an IgG. The antibody or antibody fragment may recognize an epitope on RSV F protein in antigenic site II. The antibody or antibody fragment may escape competition with non-neutralizing site II antibodies. The antibody or antibody fragment may be administered prior to infection, or after infection.

The antibody or antibody fragment may be encoded by clone-paired variable sequences as set forth in Table 1, may be encoded by light and heavy chain variable sequences having 70%, 80%, or 90% identity to clone-paired variable sequences as set forth in Table 1, or may be encoded by light and heavy chain variable sequences having 95% identity to clone-paired sequences as set forth in Table 1. The antibody or antibody fragment may comprise light and heavy chain variable sequences according to clone-paired sequences from Table 2, may comprise light and heavy chain variable sequences having 70%, 80% or 90% identity to clone-paired sequences from Table 2 and may comprise light and heavy chain variable sequences having 95% identity to clone-paired sequences from Table 2. Delivering may comprises antibody or antibody fragment administration, or genetic delivery with an RNA or DNA sequence or vector encoding the antibody or antibody fragment.

In another embodiment, there is provided a monoclonal antibody, wherein the antibody or antibody fragment is characterized by clone-paired heavy and light chain CDR sequences from Tables 3 and 4, respectively. The antibody or antibody fragment may be encoded by clone-paired variable sequences as set forth in Table 1, may be encoded by light and heavy chain variable sequences having 70%, 80%, or 90% identity to clone-paired variable sequences as set forth in Table 1, or may be encoded by light and heavy chain variable sequences having 95% identity to clone-paired sequences as set forth in Table 1. The antibody or antibody fragment may comprise light and heavy chain variable sequences according to clone-paired sequences from Table 2, may comprise light and heavy chain variable sequences having 70%, 80% or 90% identity to clone-paired sequences from Table 2 and may comprise light and heavy chain variable sequences having 95% identity to clone-paired sequences from Table 2.

The antibody fragment may be a recombinant ScFv (single chain fragment variable) antibody, Fab fragment, F(ab′)₂ fragment, or Fv fragment. The antibody may be a chimeric antibody, a bispecific antibody, and/or is an IgG. The antibody or antibody fragment may recognize an epitope on RSV F protein in antigenic site II, and optionally escapes competition with non-neutralizing site II antibodies. The antibody or antibody fragment may further comprise a cell penetrating peptide and/or is an intrabody.

In still another embodiment, there is provided a hybridoma or engineered cell encoding an antibody or antibody fragment wherein the antibody or antibody fragment is characterized by clone-paired heavy and light chain CDR sequences from Tables 3 and 4, respectively. The hybridoma or engineered cell may encode clone-paired variable sequences as set forth in Table 1, may encode by light and heavy chain variable sequences having 70%, 80%, or 90% identity to clone-paired variable sequences as set forth in Table 1, or may encode by light and heavy chain variable sequences having 95% identity to clone-paired sequences as set forth in Table 1. The hybridoma or engineered cell may express light and heavy chain variable sequences according to clone-paired sequences from Table 2, may express light and heavy chain variable sequences having 70%, 80% or 90% identity to clone-paired sequences from Table 2, and may express light and heavy chain variable sequences having 95% identity to clone-paired sequences from Table 2.

The hybridoma or engineered cell may express an antibody fragment that is a recombinant ScFv (single chain fragment variable) antibody, Fab fragment, F(ab′)₂ fragment, or Fv fragment. The hybridoma or engineered cell may express a chimeric antibody, a bispecific antibody, and/or is an IgG. The hybridoma or engineered cell may express an antibody or antibody fragment that recognizes an epitope on RSV F protein in antigenic site II, and optionally escapes competition with non-neutralizing site II antibodies. The hybridoma or engineered cell may produce an antibody or antibody fragment that further comprises a cell penetrating peptide and/or is an intrabody.

In a further embodiment, there is provided a vaccine formulation comprising one or more antibodies or antibody fragments characterized by clone-paired heavy and light chain CDR sequences from Tables 3 and 4, respectively. The vaccine formulation may comprise antibodies or antibody fragments encoded by light and heavy chain variable sequences according to clone-paired sequences from Table 1, encoded by light and heavy chain variable sequences having at least 70%, 80%, or 90% identity to clone-paired sequences from Table 1, or encoded by light and heavy chain variable sequences having at least 95% identity to clone-paired sequences from Table 1. The vaccine formulation may comprise antibodies or antibody fragments that comprise light and heavy chain variable sequences according to clone-paired sequences from Table 2, may express light and heavy chain variable sequences having 70%, 80% or 90% identity to clone-paired sequences from Table 2, or that comprise light and heavy chain variable sequences having 95% identity to clone-paired sequences from Table 2.

The vaccine formulation may comprise antibody fragments such as a recombinant ScFv (single chain fragment variable) antibody, Fab fragment, F(ab′)₂ fragment, or Fv fragment, or a chimeric antibody, a bispecific antibody, or an IgG. The vaccine formulation may comprises antibody or antibody fragment that recognize an epitope on RSV F protein in antigenic site II, and optionally escapes competition with non-neutralizing site II antibodies. The vaccine formulation may comprise antibodies or antibody fragments further comprises a cell penetrating peptide and/or is an intrabody.

In yet a further embodiment, there is provided a method of identifying an anti-human respiratory syncytial virus (hRSV) protein F site II-specific neutralizing antibody comprising (a) contacting a candidate antibody with hRSV protein F in the presence of a known site II-specific neutralizing antibody or antigen binding fragment thereof (b) assessing binding of said candidate antibody to hRSV protein F; and (c) identifying said candidate antibody as a protein F site II-specific neutralizing antibody when said known site II-specific neutralizing antibody or antigen binding fragment thereof blocks binding of said candidate antibody to hRSV protein F. The method may further comprise performing a control reaction where said candidate antibody is contacted with hRSV protein F in the absence of a known site II-specific neutralizing antibody or fragment thereof. Detection may comprise ELISA, RIA or Western blot. The known site II-specific neutralizing antibody or fragment thereof may be encoded by clone-paired variable sequences as set forth in Table 1, may be encoded by light and heavy chain variable sequences having 70%, 80%, or 90% identity to clone-paired variable sequences as set forth in Table 1, or may be encoded by light and heavy chain variable sequences having 95% identity to clone-paired sequences as set forth in Table 1. The known site II-specific neutralizing antibody or fragment thereof may comprise light and heavy chain variable sequences according to clone-paired sequences from Table 2, may comprise light and heavy chain variable sequences having 70%, 80% or 90% identity to clone-paired sequences from Table 2, or may comprise light and heavy chain variable sequences having 95% identity to clone-paired sequences from Table 2. The antigen fragment may be a recombinant ScFv (single chain fragment variable) antibody, Fab fragment, F(ab′)₂ fragment, or Fv fragment.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

The patent or application file contains at least one drawing executed in color. Copies of this patent or patent application publication with color drawing(s) will be provided by the Office upon request and payment of the necessary fee.

FIGS. 1A-E. Epitope binning and saturation alanine scanning mutagenesis for mAbs binding RSV F protein in the post-fusion (FIG. 1A) or DS-Cav1 pre-fusion (FIG. 1B) conformations. Data indicate the percent binding of the competing antibody in the presence of the primary antibody, as compared to the competing antibody alone. Cells filled in black indicate full competition, in which <33% of the un-competed signal was observed, intermediate competition (grey) if signal was between 33-66%, and non-competing (white) if signal was >66%. Antigenic sites are highlighted at the top and side based on competition-binding with the control mAbs D25 (site Ø), 131-2a (site I), palivizumab (PALI) or motavizumab (MOTA) (site II) or 101F (site IV). Competition for antigenic site II mAbs formed three groups, corresponding to site VII (center black/grey box), IIa (box between sites VII and IIb), or IIb (lower right black box). Competition with non-neutralizing mAbs was less pronounced in the pre-fusion conformation. (FIG. 1C) Binding values for isolated mAbs 14N4 and 12I1 with palivizumab or D25 control mAbs. The mAb reactivity for each RSV F mutation was calculated relative to that of wild-type RSV F. Error bars indicate standard deviations. (FIG. 1D) The residues important for binding of 14N4 or 12I1 are mapped on the RSV F trimeric structure as spheres. Residues important for 14N4 and 12I1 binding are very distant on the same protomer, yet are in close contact through quaternary interactions at the protomer 1-protomer 2 interface, leading to competition between neutralizing mAb 14N4 and non-neutralizing mAb 12I1. (FIG. 1E) Quaternary interactions between antigenic sites IIa and VII were less pronounced in the pre-fusion conformation, as site IIa is farther away from site VII on the same and adjacent protomers.

FIGS. 2A-D. The complex of mAb 14N4 with RSV F. (FIG. 2A) X-ray crystal structure of Fab 14N4 in complex with post-fusion RSV strain A2 F protein. The overall structure is displayed in surface form and rotated 90° in cartoon form. MAb 14N4 bound RSV F at each protomer in the trimeric structure. EM class averages with RSV 18537 B are also displayed, confirming the binding location of 14N4-Fab. The side length of panels is 32.7 nm. (FIG. 2B) Chemical interactions between Fab 14N4 and RSV strain A2 F protein. Several key hydrogen bonds are important for molecular recognition. (FIG. 2C) Overlay of the complex with the motavizumab-site II peptide complex (PDB: 3IXT). Motavizumab binds antigenic site II at a different orientation than mAb 14N4, allowing it to be free of interactions with site VII. (FIG. 2D) Interactions between motavizumab and the antigenic site II peptide (PDB: 3IXT). Lys271 does not interact with motavizumab, unlike its role in the 14N4-RSV F complex.

FIGS. 3A-C. Human mAbs bind to synthetic immunogens. (FIG. 3A) X-ray structure of FFL_001 displayed with RSV antigenic site VII (PDB: 4JLR). A model of RPM-1 shows the region surrounding the corresponding antigenic site VII in the MPV F protein, and RSV antigenic site VII. (FIG. 3B) ELISA binding curves for three human mAbs 14N4, 13A8, and 3J20 along with antigenic site VII mAbs motavizumab and palivizumab. Binding curves for FFL_001 are solid circles and for RPM-1 are open boxes. Binding to MPV F protein is solid boxes. EC₅₀ values are displayed for each, in corresponding colors. Error bars indicate 95% confidence intervals. (FIG. 3C) Surface plasmon resonance of 14N4, 13A8, and 3J20 Fabs binding to FFL_001 with calculated K_{D} values displayed. Data points are overlaid with the curve fit line in solid black. Dotted lines indicate the start of association and dissociation steps.

FIGS. 4A-C. Hydrogen deuterium exchange with FFL_001 and comparison with mab 17HD9. (FIG. 4A) HD exchange protection of 14N4 upon scaffold binding (SEQ ID NO: 92). Each antibody-derived peptide was monitored for deuterium incorporation in the presence or absence of the scaffold protein. Peptides are colored according to the difference in incorporated deuterium atoms in the bound versus unbound form, with a large reduction in incorporation indicating a putative binding site. Values from the 30 minute deuteration time point are shown. HD exchange profile of 14N4-derived peptides is mapped onto the 14N4 Fab structure. (FIG. 4B) Interactions between the macaque mAb 17HD9 and FFL_001 (PDB: 4N9G). (FIG. 4C) Overlay of 14N4 with antigenic site II and 17-HD9 with FFL_001. 14N4 and 17HD9 (PDB: 4N9G) are shown. 17HD9 interacts with the lower loop of antigenic site II along with both helices, while 14N4 interacts only with the two helices.

FIG. 5. Neutralization curves for the isolated mAbs. IC₅₀ values are displayed in Table 5. An Ebola virus-specific mAb EBOV284 was included as a control. Error bars represent 95% confidence intervals.

FIG. 6. ELISA binding curves for the isolated mAbs and controls to RSV F protein strain and construct variants. EC₅₀ values for these curves are displayed in Table 5. West Nile virus envelope (Env) protein was used as a negative control. Error bars represent 95% confidence intervals for mAb neutralization experiments, and SEM for serum neutralization experiments.

FIGS. 7A-C. (FIG. 7A) Palivizumab competition assay for donor serum, and (FIG. 7B) for mAbs 12I1 and 14N4. Increasing donor serum or mAb concentration reduces the signal from biotinylated palivizumab. Competition was not detected between 12I1 and palivizumab on pre-fusion RSV F, confirming the observation in epitope binning, as 12I1 favors the post-fusion F conformation. (FIG. 7C) Competition neutralization assays where RSV A2 was incubated initially with 50 μg/mL mAb 12I1 revealed that site VII mAbs do not block neutralization of 14N4 or palivizumab. All error bars represent 95% confidence intervals.

FIGS. 8A-C. Structural differences between the CH1 region of free 14N4-Fab and the 14N4-Fab-RSV F complex. (FIG. 8A) Overlay of crystal structures of 14N4-Fab and 14N4-Fab-RSV F complex. The CH1 region of 14N4-Fab is shifted upward in the complex. (FIG. 8B) Symmetry partners of the 14N4-Fab-RSV F complex. (FIG. 8C) Interactions between symmetry-related 14N4-Fab CH1 regions, to which is attributed the shift in the CH1 region from free 14N4 Fab.

FIG. 9. Stereo-view of the region surrounding the 14N4-Fab/RSV F interface. The composite omit electron density map is contoured to 2.0 σ. Density surrounding the residues in this region is well-ordered, allowing for accurate determination of the atomic positions in the CDR loops and antigenic site II.

FIG. 10. Surface plasmon resonance control binding experiments using mutated FFL_001. Fabs do not bind FFL_001 with R33C, N72Y, and K82E mutations.

FIGS. 11A-B. Sequence coverage and individual HD exchange plots of 14N4 Fab. (FIG. 11A) Peptide coverage map of 14N4 (SEQ ID NO: 92). Each analyzed peptide is depicted as a solid line beneath the sequence. CDR loops are highlighted above the sequence. (FIG. 11B) Two deuterium uptake profile examples for peptides analyzed, both in the apo (circle) or antigen-bound (box) forms (SEQ ID NO: 93, upper; SEQ ID NO: 94, lower). Deuterium uptake was measured as a percentage of the theoretical maximum. Peptides were deuterated for either 0, 15, 30, or 60 min. Error bars represent the standard deviation of three replicates.

FIG. 12. Epitope binning for select RSV F mAbs with macaque mAb 17HD9. 17HD9 competes with site VII mAbs similar to 14N4 and palivizumab, and also competes with 101F. Data indicate the percent binding of the competing antibody in the presence of the primary antibody, as compared to the competing antibody alone. Cells filled in black indicate full competition, in which ≤33% of the un-competed signal was observed, intermediate competition (grey) if signal was between 33-66%, and non-competing (white) if signal was 66%.

FIG. 13. IMGT and Kabat numbering for heavy chain and light chain junction regions (SEQ ID NOs: 95 and 96, respectively).

### DESCRIPTION OF ILLUSTRATIVE EMBODIMENTS

Respiratory syncytial virus is a highly contagious human pathogen, infecting the majority of infants before age two, and is the leading cause of viral bronchiolitis and viral pneumonia in infants and children. An approved prophylactic humanized mouse monoclonal antibody, palivizumab, is currently the standard-of-care treatment for immunocompromised individuals, and competition with palivizumab has been proposed as the basis for measuring effective immune responses for vaccine trials.

In order to characterize the human immune response to the RSV F protein, the inventors isolated and characterized human mAbs targeting the RSV F protein, and in particular focused discovery efforts on antigenic site II. Using a combination of X-ray crystallography, hydrogen-deuterium exchange, and saturation alanine mutagenesis scanning, the inventors show the structural basis for neutralization by a human antibody at the palivizumab antigenic site. Furthermore, the inventors describe non-neutralizing antibodies that directly compete with palivizumab and another human antibody 14N4. Defining the structural basis for interaction of site II-specific antibodies revealed new insights into the complexity of this site and diverse modes of recognition that determined whether or not site II human antibodies neutralize RSV. Taken together, the data presented provide new concepts in structure-based vaccine design. These and other aspects of the disclosure are described in detail below.

### I. RESPIRATORY SYNCYTIAL VIRUS

Human respiratory syncytial virus (RSV) is a syncytial virus that causes respiratory tract infections. It is a major cause of lower respiratory tract infections and hospital visits during infancy and childhood. A prophylactic medication, palivizumab, can be employed to prevent human RSV in preterm (under 35 weeks gestation) infants, infants with certain congenital heart defects (CHD) or bronchopulmonary dysplasia (BPD), and infants with congenital malformations of the airway. Treatment is limited to supportive care (e.g., C-PAP), including oxygen therapy.

Human RSV is a negative-sense, single-stranded RNA virus of the family Pneumoviridae. Its name comes from the fact that F proteins on the surface of the virus cause the cell membranes on nearby cells to merge, forming syncytia. It was first isolated in 1956 from a chimpanzee, and called Chimpanzee Coryza Agent (CCA). Also in 1956, a new type of cytopathogenic myxovirus was isolated from a group of human infants with infantile croup.

In temperate climates there is an annual epidemic during the winter months. In tropical climates, infection is most common during the rainy season. In the United States, 60% of infants are infected during their first RSV season, and nearly all children will have been infected with the virus by 2-3 years of age. Of those infected with RSV, 2-3% will develop bronchiolitis, necessitating hospitalization. Natural infection with HRSV induces protective immunity which wanes over time—possibly more so than other respiratory viral infections—and thus people can be infected multiple times. Sometimes an infant can become symptomatically infected more than once, even within a single HRSV season. Severe HRSV infections have increasingly been found among elderly patients. Young adults can be re-infected every five to seven years, with symptoms looking like a sinus infection or a cold (infections can also be asymptomatic).

The incubation time (from infection until symptoms arrive) is 4-5 days. For adults, HRSV produces mainly mild symptoms, often indistinguishable from common colds and minor illnesses. The Centers for Disease Control consider HRSV to be the “most common cause of bronchiolitis (inflammation of the small airways in the lung) and pneumonia in children under 1 year of age in the United States.” For some children, RSV can cause bronchiolitis, leading to severe respiratory illness requiring hospitalization and, rarely, causing death. This is more likely to occur in patients that are immunocompromised or infants born prematurely. Other HRSV symptoms common among infants include listlessness, poor or diminished appetite, and a possible fever.

Recurrent wheezing and asthma are more common among individuals who suffered severe HRSV infection during the first few months of life than among controls; whether HRSV infection sets up a process that leads to recurrent wheezing or whether those already predisposed to asthma are more likely to become severely ill with HRSV has yet to be determined.

Symptoms of pneumonia in immuno-compromised patients such as in transplant patients and especially bone marrow transplant patients should be evaluated to rule out HRSV infection. This can be done by means of polymerase chain reaction (PCR) testing for HRSV nucleic acids in peripheral blood samples if all other infectious processes have been ruled out or if it is highly suspicious for RSV such as a recent exposure to a known source of HRSV infection.

Complications include bronchiolitis or pneumonia, asthma, recurring infections, and acute otitis media.

Transmission. The incubation period is 2-8 days, but is usually 4-6 days. RSV spreads easily by direct contact, and can remain viable for a half an hour or more on hands or for up to 5 hours on countertops. Childcare facilities allow for rapid child-to-child transmission in a short period of time. RSV can last 2-8 days, but symptoms may persist for up to three weeks.

The human RSV is virtually the same as chimpanzee coryza virus and can be transmitted from apes to humans, although transmission from humans to apes is more common. The virus has also been recovered from cattle, goats and sheep, but these are not regarded as major vectors of transmission and there is no animal reservoir of the virus.

Virology. Human RSV is a medium-sized (120-200 nm) enveloped virus that contains a lipoprotein coat and a linear negative-sense RNA genome (must be converted to an anti-sense genome prior to translation). The former contains virally encoded F, G, and SH lipoproteins. The F and G lipoproteins are the only two that target the cell membrane, and are highly conserved among RSV isolates. HRSV is divided into two antigenic subgroups, A and B, on the basis of the reactivity of the virus with monoclonal antibodies against the attachment (G) and fusion (F) glycoproteins. Subtype B is characterized as the asymptomatic strains of the virus that the majority of the population experiences. The more severe clinical illnesses involve subtype A strains, which tend to predominate in most outbreaks.

The genome is ˜15,000 nucleotides in length and is composed of a single strand of RNA with negative polarity. It has 10 genes encoding 11 proteins. To date, 10 HRSV-A genotypes have been designated, GA1 to GA7, SAA1, NA1, and NA2. The HRSV-B genotypes include GB1 to GB4, SAB1 to SAB3, and BA1 to BA6. The genome of HRSV was completely sequenced in 1997.

Diagnosis. Human respiratory syncytial virus may be suspected based on the time of year of the infection; prevalence usually coincides with the winter flu season. Tests include (a) chest X-rays to check for typical bilateral perihilar fullness of bronchiolitis induced by the virus, (b) skin monitoring to check for hypoxemia, a lower than usual level of oxygen in the bloodstream, (c) blood tests to check white cell counts or to look for the presence of viruses, bacteria or other organisms, and (d) lab testing of respiratory secretions.

Several different types of laboratory tests are commercially available for diagnosis of RSV infection. Rapid diagnostic assays performed on respiratory specimens are available commercially. Most clinical laboratories currently utilize antigen detection tests. Compared with culture, the sensitivity of antigen detection tests generally ranges from 80% to 90%. Antigen detection tests and culture are generally reliable in young children but less useful in older children and adults.

Sensitivity of virus isolation from respiratory secretions in cell culture varies among laboratories. RT-PCR assays are now commercially available. The sensitivity of these assays is equal to or exceeds the sensitivity of virus isolation and antigen detections methods. Highly sensitive RT-PCR assays should be considered when testing adults, because they may have low viral loads in their respiratory specimens.

Serologic tests are less frequently used for diagnosis. Although useful for research, a diagnosis using a collection of paired acute and convalescent sera to demonstrate a significant rise in antibody titer to HRSV cannot be made in time to guide care of the patient. On top of that, the antibody level does not always correlate with the acuteness or activity level of the infection.

RSV infection can be confirmed using tests for antigens or antibodies, or viral RNA by reverse transcription PCR. Quantification of viral load can be determined by various assay tests.

Prevention. As the virus is ubiquitous in all parts of the world, avoidance of infection is not possible. However, palivizumab (brand name Synagis manufactured by Medlmmune), a moderately effective prophylactic drug, is available for infants at high risk. Palivizumab is a monoclonal antibody directed against RSV surface fusion protein. It is given by monthly injections, which are begun just prior to the RSV season and are usually continued for five months. HRSV prophylaxis is indicated for infants that are premature or have either cardiac or lung disease, but the cost of prevention limits use in many parts of the world.

Vaccine Research. A vaccine trial in 1960s using a formalin-inactivated vaccine (FI-RSV) increased disease severity in children who had been vaccinated. There is much active investigation into the development of a new vaccine, but at present no vaccine exists. Some of the most promising candidates are based on temperature sensitive mutants which have targeted genetic mutations to reduce virulence.

Scientists are attempting to develop a recombinant human respiratory syncytial virus vaccine that is suitable for intranasal instillation. Tests for determining the safety and level of resistance that can be achieved by the vaccine are being conducted in the chimpanzee, which is the only known animal that develops a respiratory illness similar to humans.

The development of a commercial human RSV vaccine has remained elusive. Recent breakthroughs have sparked continued interest in this highly sought after vaccine as the annual medical burden relating to human RSV has remained high, equal to Influenza and Pneumococcus.

Treatment. To date, treatment has been limited to supportive measures. Adrenaline, bronchodilators, steroids, antibiotics, and ribavirin confer “no real benefit.” Studies of nebulized hypertonic saline have shown that the use of nebulized 3% HS is a safe, inexpensive, and effective treatment for infants hospitalized with moderately severe viral bronchiolitis where respiratory syncytial virus (RSV) accounts for the majority of viral bronchiolitis cases. One study noted a 26% reduction in length of stay: 2.6±1.9 days, compared with 3.5±2.9 days in the normal-saline treated group (p=0.05). Supportive care includes fluids and oxygen until the illness runs its course. Salbutamol may be used in an attempt to relieve any bronchospasm if present. Increased airflow, humidified and delivered via nasal cannula, may be supplied in order to reduce the effort required for respiration.

### II. MONOCLONAL ANTIBODIES AND PRODUCTION THEREOF

A. General Methods

It will be understood that monoclonal antibodies binding to Human respiratory syncytial virus will have several applications. These include the production of diagnostic kits for use in detecting and diagnosing Human respiratory syncytial virus infection, as well as for treating the same. In these contexts, one may link such antibodies to diagnostic or therapeutic agents, use them as capture agents or competitors in competitive assays, or use them individually without additional agents being attached thereto. The antibodies may be mutated or modified, as discussed further below. Methods for preparing and characterizing antibodies are well known in the art (see, e.g., Antibodies: A Laboratory Manual, Cold Spring Harbor Laboratory, 1988; U.S. Pat. No. 4,196,265).

The methods for generating monoclonal antibodies (MAbs) generally begin along the same lines as those for preparing polyclonal antibodies. The first step for both these methods is immunization of an appropriate host or identification of subjects who are immune due to prior natural infection. As is well known in the art, a given composition for immunization may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimyde and bis-biazotized benzidine. As also is well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

In the case of human antibodies against natural pathogens, a suitable approach is to identify subjects that have been exposed to the pathogens, such as those who have been diagnosed as having contracted the disease, or those who have been vaccinated to generate protective immunity against the pathogen. Circulating anti-pathogen antibodies can be detected, and antibody producing B cells from the antibody-positive subject may then be obtained.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster injection, also may be given. The process of boosting and titering is repeated until a suitable titer is achieved. When a desired level of immunogenicity is obtained, the immunized animal can be bled and the serum isolated and stored, and/or the animal can be used to generate MAbs.

Following immunization, somatic cells with the potential for producing antibodies, specifically B lymphocytes (B cells), are selected for use in the MAb generating protocol. These cells may be obtained from biopsied spleens or lymph nodes, or from circulating blood. The antibody-producing B lymphocytes from the immunized animal are then fused with cells of an immortal myeloma cell, generally one of the same species as the animal that was immunized or human or human/mouse chimeric cells. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas). Any one of a number of myeloma cells may be used, as are known to those of skill in the art (Goding, pp. 65-66, 1986; Campbell, pp. 75-83, 1984).

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. Fusion methods using Sendai virus have been described by Kohler and Milstein (1975; 1976), and those using polyethylene glycol (PEG), such as 37% (v/v) PEG, by Gefter et al. (1977). The use of electrically induced fusion methods also is appropriate (Goding, pp. 71-74, 1986). Fusion procedures usually produce viable hybrids at low frequencies, about 1×10⁻⁶ to 1×10⁻⁸. However, this does not pose a problem, as the viable, fused hybrids are differentiated from the parental, infused cells (particularly the infused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine. Ouabain is added if the B cell source is an Epstein Barr virus (EBV) transformed human B cell line, in order to eliminate EBV transformed lines that have not fused to the myeloma.

The preferred selection medium is HAT or HAT with ouabain. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells. When the source of B cells used for fusion is a line of EBV-transformed B cells, as here, ouabain may also be used for drug selection of hybrids as EBV-transformed B cells are susceptible to drug killing, whereas the myeloma partner used is chosen to be ouabain resistant.

Culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays dot immunobinding assays, and the like. The selected hybridomas are then serially diluted or single-cell sorted by flow cytometric sorting and cloned into individual antibody-producing cell lines, which clones can then be propagated indefinitely to provide mAbs. The cell lines may be exploited for MAb production in two basic ways. A sample of the hybridoma can be injected (often into the peritoneal cavity) into an animal (e.g., a mouse). Optionally, the animals are primed with a hydrocarbon, especially oils such as pristane (tetramethylpentadecane) prior to injection. When human hybridomas are used in this way, it is optimal to inject immunocompromised mice, such as SCID mice, to prevent tumor rejection. The injected animal develops tumors secreting the specific monoclonal antibody produced by the fused cell hybrid. The body fluids of the animal, such as serum or ascites fluid, can then be tapped to provide MAbs in high concentration. The individual cell lines could also be cultured in vitro, where the MAbs are naturally secreted into the culture medium from which they can be readily obtained in high concentrations. Alternatively, human hybridoma cells lines can be used in vitro to produce immunoglobulins in cell supernatant. The cell lines can be adapted for growth in serum-free medium to optimize the ability to recover human monoclonal immunoglobulins of high purity.

MAbs produced by either means may be further purified, if desired, using filtration, centrifugation and various chromatographic methods such as FPLC or affinity chromatography. Fragments of the monoclonal antibodies of the disclosure can be obtained from the purified monoclonal antibodies by methods which include digestion with enzymes, such as pepsin or papain, and/or by cleavage of disulfide bonds by chemical reduction. Alternatively, monoclonal antibody fragments encompassed by the present disclosure can be synthesized using an automated peptide synthesizer.

It also is contemplated that a molecular cloning approach may be used to generate monoclonals. For this, RNA can be isolated from the hybridoma line and the antibody genes obtained by RT-PCR and cloned into an immunoglobulin expression vector. Alternatively, combinatorial immunoglobulin phagemid libraries are prepared from RNA isolated from the cell lines and phagemids expressing appropriate antibodies are selected by panning using viral antigens. The advantages of this approach over conventional hybridoma techniques are that approximately 10⁴ times as many antibodies can be produced and screened in a single round, and that new specificities are generated by H and L chain combination which further increases the chance of finding appropriate antibodies.

Other U.S. patents, each incorporated herein by reference, that teach the production of antibodies useful in the present disclosure include U.S. Pat. No. 5,565,332, which describes the production of chimeric antibodies using a combinatorial approach; U.S. Pat. No. 4,816,567 which describes recombinant immunoglobulin preparations; and U.S. Pat. No. 4,867,973 which describes antibody-therapeutic agent conjugates.

B. Antibodies of the Present Disclosure

Antibodies according to the present disclosure may be defined, in the first instance, by their binding specificity. Those of skill in the art, by assessing the binding specificity/affinity of a given antibody using techniques well known to those of skill in the art, can determine whether such antibodies fall within the scope of the instant claims. In one aspect, there are provided monoclonal antibodies having clone-paired CDR's from the heavy and light chains as illustrated in Tables 3 and 4, respectively. Such antibodies may be produced by the clones discussed below in the Examples section using methods described herein.

In a second aspect, the antibodies may be defined by their variable sequence, which include additional “framework” regions. These are provided in Tables 1 and 2 that encode or represent full variable regions. Furthermore, the antibodies sequences may vary from these sequences, optionally using methods discussed in greater detail below. For example, nucleic acid sequences may vary from those set out above in that (a) the variable regions may be segregated away from the constant domains of the light and heavy chains, (b) the nucleic acids may vary from those set out above while not affecting the residues encoded thereby, (c) the nucleic acids may vary from those set out above by a given percentage, e.g., 70%, 75%, 80%, 85%, 90%, 91%, 92%, 93%, 94%, 95%, 96%, 97%, 98% or 99% homology, (d) the nucleic acids may vary from those set out above by virtue of the ability to hybridize under high stringency conditions, as exemplified by low salt and/or high temperature conditions, such as provided by about 0.02 M to about 0.15 M NaCl at temperatures of about 50° C. to about 70° C., (e) the amino acids may vary from those set out above by a given percentage, e.g., 80%, 85%, 90%, 91%, 92%, 93%, 94%, 95%, 96%, 97%, 98% or 99% homology, or (f) the amino acids may vary from those set out above by permitting conservative substitutions (discussed below). Each of the foregoing applies to the nucleic acid sequences set forth as Table 1 and the amino acid sequences of Table 2.

C. Engineering of Antibody Sequences

In various embodiments, one may choose to engineer sequences of the identified antibodies for a variety of reasons, such as improved expression, improved cross-reactivity or diminished off-target binding. The following is a general discussion of relevant techniques for antibody engineering.

Hybridomas may be cultured, then cells lysed, and total RNA extracted. Random hexamers may be used with RT to generate cDNA copies of RNA, and then PCR performed using a multiplex mixture of PCR primers expected to amplify all human variable gene sequences. PCR product can be cloned into pGEM-T Easy vector, then sequenced by automated DNA sequencing using standard vector primers. Assay of binding and neutralization may be performed using antibodies collected from hybridoma supernatants and purified by FPLC, using Protein G columns.

Recombinant full length IgG antibodies were generated by subcloning heavy and light chain Fv DNAs from the cloning vector into an IgG plasmid vector, transfected into 293 Freestyle cells or CHO cells, and antibodies were collected an purified from the 293 or CHO cell supernatant.

The rapid availability of antibody produced in the same host cell and cell culture process as the final cGMP manufacturing process has the potential to reduce the duration of process development programs. Lonza has developed a generic method using pooled transfectants grown in CDACF medium, for the rapid production of small quantities (up to 50 g) of antibodies in CHO cells. Although slightly slower than a true transient system, the advantages include a higher product concentration and use of the same host and process as the production cell line. Example of growth and productivity of GS-CHO pools, expressing a model antibody, in a disposable bioreactor: in a disposable bag bioreactor culture (5 L working volume) operated in fed-batch mode, a harvest antibody concentration of 2 g/L was achieved within 9 weeks of transfection.

Antibody molecules will comprise fragments (such as F(ab′), F(ab′)₂) that are produced, for example, by the proteolytic cleavage of the mAbs, or single-chain immunoglobulins producible, for example, via recombinant means. Such antibody derivatives are monovalent. In one embodiment, such fragments can be combined with one another, or with other antibody fragments or receptor ligands to form “chimeric” binding molecules. Significantly, such chimeric molecules may contain substituents capable of binding to different epitopes of the same molecule.

In related embodiments, the antibody is a derivative of the disclosed antibodies, e.g., an antibody comprising the CDR sequences identical to those in the disclosed antibodies (e.g., a chimeric, or CDR-grafted antibody). Alternatively, one may wish to make modifications, such as introducing conservative changes into an antibody molecule. In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: basic amino acids: arginine (+3.0), lysine (+3.0), and histidine (−0.5); acidic amino acids: aspartate (+3.0±1), glutamate (+3.0±1), asparagine (+0.2), and glutamine (+0.2); hydrophilic, nonionic amino acids: serine (+0.3), asparagine (+0.2), glutamine (+0.2), and threonine (−0.4), sulfur containing amino acids: cysteine (−1.0) and methionine (−1.3); hydrophobic, nonaromatic amino acids: valine (−1.5), leucine (−1.8), isoleucine (−1.8), proline (−0.5±1), alanine (−0.5), and glycine (0); hydrophobic, aromatic amino acids: tryptophan (−3.4), phenylalanine (−2.5), and tyrosine (−2.3).

It is understood that an amino acid can be substituted for another having a similar hydrophilicity and produce a biologically or immunologically modified protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

The present disclosure also contemplates isotype modification. By modifying the Fc region to have a different isotype, different functionalities can be achieved. For example, changing to IgG₁ can increase antibody dependent cell cytotoxicity, switching to class A can improve tissue distribution, and switching to class M can improve valency. Modifications in the Fc region can be introduced to extend the in vivo half-life of the antibody, or to alter Fc mediated functions such as complement activation, antibody dependent cellular cytotoxicity (ADCC), and FcR mediated phagocytosis.

Other types of modifications include residue modification designed to reduce oxidation, aggregation, deamidation, and immunogenicity in humans. Other changes can lead to an increase in manufacturability or yield, or reduced tissue cross-reactivity in humans.

Modified antibodies may be made by any technique known to those of skill in the art, including expression through standard molecular biological techniques, or the chemical synthesis of polypeptides. Methods for recombinant expression are addressed elsewhere in this document.

D. Single Chain Antibodies

A Single Chain Variable Fragment (scFv) is a fusion of the variable regions of the heavy and light chains of immunoglobulins, linked together with a short (usually serine, glycine) linker. This chimeric molecule retains the specificity of the original immunoglobulin, despite removal of the constant regions and the introduction of a linker peptide. This modification usually leaves the specificity unaltered. These molecules were created historically to facilitate phage display where it is highly convenient to express the antigen binding domain as a single peptide. Alternatively, scFv can be created directly from subcloned heavy and light chains derived from a hybridoma. Single chain variable fragments lack the constant Fc region found in complete antibody molecules, and thus, the common binding sites (e.g., protein A/G) used to purify antibodies. These fragments can often be purified/immobilized using Protein L since Protein L interacts with the variable region of kappa light chains.

Flexible linkers generally are comprised of helix- and turn-promoting amino acid residues such as alaine, serine and glycine. However, other residues can function as well. Tang et al. (1996) used phage display as a means of rapidly selecting tailored linkers for single-chain antibodies (scFvs) from protein linker libraries. A random linker library was constructed in which the genes for the heavy and light chain variable domains were linked by a segment encoding an 18-amino acid polypeptide of variable composition. The scFv repertoire (approx. 5×10⁶ different members) was displayed on filamentous phage and subjected to affinity selection with hapten. The population of selected variants exhibited significant increases in binding activity but retained considerable sequence diversity. Screening 1054 individual variants subsequently yielded a catalytically active scFv that was produced efficiently in soluble form. Sequence analysis revealed a conserved proline in the linker two residues after the V_{H} C terminus and an abundance of arginines and prolines at other positions as the only common features of the selected tethers.

The recombinant antibodies of the present disclosure may also involve sequences or moieties that permit dimerization or multimerization of the receptors. Such sequences include those derived from IgA, which permit formation of multimers in conjunction with the J-chain. Another multimerization domain is the Gal4 dimerization domain. In other embodiments, the chains may be modified with agents such as biotin/avidin, which permit the combination of two antibodies.

In a separate embodiment, a single-chain antibody can be created by joining receptor light and heavy chains using a non-peptide linker or chemical unit. Generally, the light and heavy chains will be produced in distinct cells, purified, and subsequently linked together in an appropriate fashion (i.e., the N-terminus of the heavy chain being attached to the C-terminus of the light chain via an appropriate chemical bridge).

Cross-linking reagents are used to form molecular bridges that tie functional groups of two different molecules, e.g., a stablizing and coagulating agent. However, it is contemplated that dimers or multimers of the same analog or heteromeric complexes comprised of different analogs can be created. To link two different compounds in a step-wise manner, hetero-bifunctional cross-linkers can be used that eliminate unwanted homopolymer formation.

An exemplary hetero-bifunctional cross-linker contains two reactive groups: one reacting with primary amine group (e.g., N-hydroxy succinimide) and the other reacting with a thiol group (e.g., pyridyl disulfide, maleimides, halogens, etc.). Through the primary amine reactive group, the cross-linker may react with the lysine residue(s) of one protein (e.g., the selected antibody or fragment) and through the thiol reactive group, the cross-linker, already tied up to the first protein, reacts with the cysteine residue (free sulfhydryl group) of the other protein (e.g., the selective agent).

It is preferred that a cross-linker having reasonable stability in blood will be employed. Numerous types of disulfide-bond containing linkers are known that can be successfully employed to conjugate targeting and therapeutic/preventative agents. Linkers that contain a disulfide bond that is sterically hindered may prove to give greater stability in vivo, preventing release of the targeting peptide prior to reaching the site of action. These linkers are thus one group of linking agents.

Another cross-linking reagent is SMPT, which is a bifunctional cross-linker containing a disulfide bond that is “sterically hindered” by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the target site.

The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido) ethyl-1,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane (Wawrzynczak & Thorpe, 1987). The use of such cross-linkers is well understood in the art. Another embodiment involves the use of flexible linkers.

U.S. Pat. No. 4,680,338, describes bifunctional linkers useful for producing conjugates of ligands with amine-containing polymers and/or proteins, especially for forming antibody conjugates with chelators, drugs, enzymes, detectable labels and the like. U.S. Pat. Nos. 5,141,648 and 5,563,250 disclose cleavable conjugates containing a labile bond that is cleavable under a variety of mild conditions. This linker is particularly useful in that the agent of interest may be bonded directly to the linker, with cleavage resulting in release of the active agent. Particular uses include adding a free amino or free sulfhydryl group to a protein, such as an antibody, or a drug.

U.S. Pat. No. 5,856,456 provides peptide linkers for use in connecting polypeptide constituents to make fusion proteins, e.g., single chain antibodies. The linker is up to about 50 amino acids in length, contains at least one occurrence of a charged amino acid (preferably arginine or lysine) followed by a proline, and is characterized by greater stability and reduced aggregation. U.S. Pat. No. 5,880,270 discloses aminooxy-containing linkers useful in a variety of immunodiagnostic and separative techniques.

E. Intrabodies

In a particular embodiment, the antibody is a recombinant antibody that is suitable for action inside of a cell—such antibodies are known as “intrabodies.” These antibodies may interfere with target function by a variety of mechanism, such as by altering intracellular protein trafficking, interfering with enzymatic function, and blocking protein-protein or protein-DNA interactions. In many ways, their structures mimic or parallel those of single chain and single domain antibodies, discussed above. Indeed, single-transcript/single-chain is an important feature that permits intracellular expression in a target cell, and also makes protein transit across cell membranes more feasible. However, additional features are required.

The two major issues impacting the implementation of intrabody therapeutic are delivery, including cell/tissue targeting, and stability. With respect to delivery, a variety of approaches have been employed, such as tissue-directed delivery, use of cell-type specific promoters, viral-based delivery and use of cell-permeability/membrane translocating peptides. With respect to the stability, the approach is generally to either screen by brute force, including methods that involve phage display and may include sequence maturation or development of consensus sequences, or more directed modifications such as insertion stabilizing sequences (e.g., Fc regions, chaperone protein sequences, leucine zippers) and disulfide replacement/modification.

An additional feature that intrabodies may require is a signal for intracellular targeting. Vectors that can target intrabodies (or other proteins) to subcellular regions such as the cytoplasm, nucleus, mitochondria and ER have been designed and are commercially available (Invitrogen Corp.; Persic et al., 1997).

By virtue of their ability to enter cells, intrabodies have additional uses that other types of antibodies may not achieve. In the case of the present antibodies, the ability to interact with the MUC1 cytoplasmic domain in a living cell may interfere with functions associated with the MUC1 CD, such as signaling functions (binding to other molecules) or oligomer formation. In particular, it is contemplated that such antibodies can be used to inhibit MUC1 dimer formation.

F. Purification

In certain embodiments, the antibodies of the present disclosure may be purified. The term “purified,” as used herein, is intended to refer to a composition, isolatable from other components, wherein the protein is purified to any degree relative to its naturally-obtainable state. A purified protein therefore also refers to a protein, free from the environment in which it may naturally occur. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include, precipitation with ammonium sulfate, PEG, antibodies and the like or by heat denaturation, followed by centrifugation; gel filtration, reverse phase, hydroxylapatite and affinity chromatography; and combinations of such and other techniques.

In purifying an antibody of the present disclosure, it may be desirable to express the polypeptide in a prokaryotic or eukaryotic expression system and extract the protein using denaturing conditions. The polypeptide may be purified from other cellular components using an affinity column, which binds to a tagged portion of the polypeptide. As is generally known in the art, it is believed that the order of conducting the various purification steps may be changed, or that certain steps may be omitted, and still result in a suitable method for the preparation of a substantially purified protein or peptide.

Commonly, complete antibodies are fractionated utilizing agents (i.e., protein A) that bind the Fc portion of the antibody. Alternatively, antigens may be used to simultaneously purify and select appropriate antibodies. Such methods often utilize the selection agent bound to a support, such as a column, filter or bead. The antibodies is bound to a support, contaminants removed (e.g., washed away), and the antibodies released by applying conditions (salt, heat, etc.).

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. Another method for assessing the purity of a fraction is to calculate the specific activity of the fraction, to compare it to the specific activity of the initial extract, and to thus calculate the degree of purity. The actual units used to represent the amount of activity will, of course, be dependent upon the particular assay technique chosen to follow the purification and whether or not the expressed protein or peptide exhibits a detectable activity.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

### III. ACTIVE/PASSIVE IMMUNIZATION AND TREATMENT/PREVENTION OF HUMAN RESPIRATORY SYNCYTIAL VIRUS INFECTION

A. Formulation and Administration

The present disclosure provides pharmaceutical compositions comprising anti-human respiratory syncytial virus antibodies and antigens for generating the same. Such compositions comprise a prophylactically or therapeutically effective amount of an antibody or a fragment thereof, or a peptide immunogen, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in “Remington's Pharmaceutical Sciences.” Such compositions will contain a prophylactically or therapeutically effective amount of the antibody or fragment thereof, preferably in purified form, together with a suitable amount of carrier so as to provide the form for proper administration to the patient. The formulation should suit the mode of administration, which can be oral, intravenous, intraarterial, intrabuccal, intranasal, nebulized, bronchial inhalation, or delivered by mechanical ventilation.

Active vaccines are also envisioned where antibodies like those disclosed are produced in vivo in a subject at risk of Human respiratory syncytial virus infection. Such vaccines can be formulated for parenteral administration, e.g., formulated for injection via the intradermal, intravenous, intramuscular, subcutaneous, or even intraperitoneal routes. Administration by intradermal and intramuscular routes are contemplated. The vaccine could alternatively be administered by a topical route directly to the mucosa, for example by nasal drops, inhalation, or by nebulizer. Pharmaceutically acceptable salts, include the acid salts and those which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Passive transfer of antibodies, known as artificially acquired passive immunity, generally will involve the use of intravenous or intramuscular injections. The forms of antibody can be human or animal blood plasma or serum, as pooled human immunoglobulin for intravenous (IVIG) or intramuscular (IG) use, as high-titer human IVIG or IG from immunized or from donors recovering from disease, and as monoclonal antibodies (MAb). Such immunity generally lasts for only a short period of time, and there is also a potential risk for hypersensitivity reactions, and serum sickness, especially from gamma globulin of non-human origin. However, passive immunity provides immediate protection. The antibodies will be formulated in a carrier suitable for injection, i.e., sterile and syringeable.

Generally, the ingredients of compositions of the disclosure are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### IV. ANTIBODY CONJUGATES

Antibodies of the present disclosure may be linked to at least one agent to from an antibody conjugate. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which have been attached to antibodies include toxins, anti-tumor agents, therapeutic enzymes, radionuclides, antiviral agents, chelating agents, cytokines, growth factors, and oligo- or polynucleotides. By contrast, a reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, photoaffinity molecules, colored particles or ligands, such as biotin.

Antibody conjugates are generally preferred for use as diagnostic agents. Antibody diagnostics generally fall within two classes, those for use in in vitro diagnostics, such as in a variety of immunoassays, and those for use in vivo diagnostic protocols, generally known as “antibody-directed imaging.” Many appropriate imaging agents are known in the art, as are methods for their attachment to antibodies (see, for e.g., U.S. Pat. Nos. 5,021,236, 4,938,948, and 4,472,509). The imaging moieties used can be paramagnetic ions, radioactive isotopes, fluorochromes, NMR-detectable substances, and X-ray imaging agents.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine²¹¹, ¹⁴carbon, ⁵¹chromium, ³⁶chlorine, ⁵⁷cobalt, ⁵⁸cobalt, copper⁶⁷, 152Eu, gallium⁶⁷, ³hydrogen, iodine¹²³, iodine¹²⁵, iodine¹³¹, indium¹¹¹, ⁵⁹iron ³²phosphorus, rhenium¹⁸⁶, rhenium¹⁸⁸, ⁷⁵selenium, ³⁵sulphur, technicium^{99m} and/or yttrium⁹⁰. ¹²⁵I is often being preferred for use in certain embodiments, and technicium^{99m} and/or indium¹¹¹ are also often preferred due to their low energy and suitability for long range detection. Radioactively labeled monoclonal antibodies of the present disclosure may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the disclosure may be labeled with technetium^{99m} by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Another type of antibody conjugates contemplated in the present disclosure are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and avidin and streptavidin compounds. The use of such labels is well known to those of skill in the art and are described, for example, in U.S. Pat. Nos. 3,817,837, 3,850,752, 3,939,350, 3,996,345, 4,277,437, 4,275,149 and 4,366,241.

Yet another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light (Potter and Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; Dholakia et al., 1989) and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. 4,472,509 and 4,938,948). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

In other embodiments, derivatization of immunoglobulins by selectively introducing sulfhydryl groups in the Fc region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature (O'Shannessy et al., 1987). This approach has been reported to produce diagnostically and therapeutically promising antibodies which are currently in clinical evaluation.

### V. IMMUNODETECTION METHODS

In still further embodiments, the present disclosure concerns immunodetection methods for binding, purifying, removing, quantifying and otherwise generally detecting Human respiratory syncytial virus and its associated antigens. While such methods can be applied in a traditional sense, another use will be in quality control and monitoring of vaccine and other virus stocks, where antibodies according to the present disclosure can be used to assess the amount or integrity (i.e., long term stability) of H1 antigens in viruses. Alternatively, the methods may be used to screen various antibodies for appropriate/desired reactivity profiles.

Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. In particular, a competitive assay for the detection and quantitation of Human respiratory syncytial virus antibodies directed to specific parasite epitopes in samples also is provided. The steps of various useful immunodetection methods have been described in the scientific literature, such as, e.g., Doolittle and Ben-Zeev (1999), Gulbis and Galand 0993), De Jager et al. (1993), and Nakamura et al. (1987). In general, the immunobinding methods include obtaining a sample suspected of containing Human respiratory syncytial virus, and contacting the sample with a first antibody in accordance with the present disclosure, as the case may be, under conditions effective to allow the formation of immunocomplexes.

These methods include methods for purifying Human respiratory syncytial virus or related antigens from a sample. The antibody will preferably be linked to a solid support, such as in the form of a column matrix, and the sample suspected of containing the Human respiratory syncytial virus or antigenic component will be applied to the immobilized antibody. The unwanted components will be washed from the column, leaving the Human respiratory syncytial virus antigen immunocomplexed to the immobilized antibody, which is then collected by removing the organism or antigen from the column.

The immunobinding methods also include methods for detecting and quantifying the amount of Human respiratory syncytial virus or related components in a sample and the detection and quantification of any immune complexes formed during the binding process. Here, one would obtain a sample suspected of containing Human respiratory syncytial virus or its antigens, and contact the sample with an antibody that binds Human respiratory syncytial virus or components thereof, followed by detecting and quantifying the amount of immune complexes formed under the specific conditions. In terms of antigen detection, the biological sample analyzed may be any sample that is suspected of containing Human respiratory syncytial virus or Human respiratory syncytial virus antigen, such as a tissue section or specimen, a homogenized tissue extract, a biological fluid, including blood and serum, or a secretion, such as feces or urine.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to Human respiratory syncytial virus or antigens present. After this time, the sample-antibody composition, such as a tissue section, ELISA plate, dot blot or Western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. Patents concerning the use of such labels include U.S. Pat. Nos. 3,817,837, 3,850,752, 3,939,350, 3,996,345, 4,277,437, 4,275,149 and 4,366,241. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two-step approach. A second binding ligand, such as an antibody that has binding affinity for the antibody, is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection uses two different antibodies. A first biotinylated antibody is used to detect the target antigen, and a second antibody is then used to detect the biotin attached to the complexed biotin. In that method, the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

A. ELISAs

Immunoassays, in their most simple and direct sense, are binding assays. Certain preferred immunoassays are the various types of enzyme linked immunosorbent assays (ELISAs) and radioimmunoassays (RIA) known in the art. Immunohistochemical detection using tissue sections is also particularly useful. However, it will be readily appreciated that detection is not limited to such techniques, and western blotting, dot blotting, FACS analyses, and the like may also be used.

In one exemplary ELISA, the antibodies of the disclosure are immobilized onto a selected surface exhibiting protein affinity, such as a well in a polystyrene microtiter plate. Then, a test composition suspected of containing the Human respiratory syncytial virus or Human respiratory syncytial virus antigen is added to the wells. After binding and washing to remove non-specifically bound immune complexes, the bound antigen may be detected. Detection may be achieved by the addition of another anti-Human respiratory syncytial virus antibody that is linked to a detectable label. This type of ELISA is a simple “sandwich ELISA.” Detection may also be achieved by the addition of a second anti-Human respiratory syncytial virus antibody, followed by the addition of a third antibody that has binding affinity for the second antibody, with the third antibody being linked to a detectable label.

In another exemplary ELISA, the samples suspected of containing the Human respiratory syncytial virus or Human respiratory syncytial virus antigen are immobilized onto the well surface and then contacted with the anti-Human respiratory syncytial virus antibodies of the disclosure. After binding and washing to remove non-specifically bound immune complexes, the bound anti-Human respiratory syncytial virus antibodies are detected. Where the initial anti-Human respiratory syncytial virus antibodies are linked to a detectable label, the immune complexes may be detected directly. Again, the immune complexes may be detected using a second antibody that has binding affinity for the first anti-Human respiratory syncytial virus antibody, with the second antibody being linked to a detectable label.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

In another embodiment, the present disclosure contemplates the use of competitive formats. This is particularly useful in the detection of Human respiratory syncytial virus antibodies in sample. In competition based assays, an unknown amount of analyte or antibody is determined by its ability to displace a known amount of labeled antibody or analyte. Thus, the quantifiable loss of a signal is an indication of the amount of unknown antibody or analyte in a sample.

Here, the inventors propose the use of labeled Human respiratory syncytial virus monoclonal antibodies to determine the amount of Human respiratory syncytial virus antibodies in a sample. The basic format would include contacting a known amount of Human respiratory syncytial virus monoclonal antibody (linked to a detectable label) with Human respiratory syncytial virus antigen or particle. The Human respiratory syncytial virus antigen or organism is preferably attached to a support. After binding of the labeled monoclonal antibody to the support, the sample is added and incubated under conditions permitting any unlabeled antibody in the sample to compete with, and hence displace, the labeled monoclonal antibody. By measuring either the lost label or the label remaining (and subtracting that from the original amount of bound label), one can determine how much non-labeled antibody is bound to the support, and thus how much antibody was present in the sample.

B. Western Blot

The Western blot (alternatively, protein immunoblot) is an analytical technique used to detect specific proteins in a given sample of tissue homogenate or extract. It uses gel electrophoresis to separate native or denatured proteins by the length of the polypeptide (denaturing conditions) or by the 3-D structure of the protein (native/non-denaturing conditions). The proteins are then transferred to a membrane (typically nitrocellulose or PVDF), where they are probed (detected) using antibodies specific to the target protein.

Samples may be taken from whole tissue or from cell culture. In most cases, solid tissues are first broken down mechanically using a blender (for larger sample volumes), using a homogenizer (smaller volumes), or by sonication. Cells may also be broken open by one of the above mechanical methods. However, it should be noted that bacteria, virus or environmental samples can be the source of protein and thus Western blotting is not restricted to cellular studies only. Assorted detergents, salts, and buffers may be employed to encourage lysis of cells and to solubilize proteins. Protease and phosphatase inhibitors are often added to prevent the digestion of the sample by its own enzymes. Tissue preparation is often done at cold temperatures to avoid protein denaturing.

The proteins of the sample are separated using gel electrophoresis. Separation of proteins may be by isoelectric point (pI), molecular weight, electric charge, or a combination of these factors. The nature of the separation depends on the treatment of the sample and the nature of the gel. This is a very useful way to determine a protein. It is also possible to use a two-dimensional (2-D) gel which spreads the proteins from a single sample out in two dimensions. Proteins are separated according to isoelectric point (pH at which they have neutral net charge) in the first dimension, and according to their molecular weight in the second dimension.

In order to make the proteins accessible to antibody detection, they are moved from within the gel onto a membrane made of nitrocellulose or polyvinylidene difluoride (PVDF). The membrane is placed on top of the gel, and a stack of filter papers placed on top of that. The entire stack is placed in a buffer solution which moves up the paper by capillary action, bringing the proteins with it. Another method for transferring the proteins is called electroblotting and uses an electric current to pull proteins from the gel into the PVDF or nitrocellulose membrane. The proteins move from within the gel onto the membrane while maintaining the organization they had within the gel. As a result of this blotting process, the proteins are exposed on a thin surface layer for detection (see below). Both varieties of membrane are chosen for their non-specific protein binding properties (i.e., binds all proteins equally well). Protein binding is based upon hydrophobic interactions, as well as charged interactions between the membrane and protein. Nitrocellulose membranes are cheaper than PVDF, but are far more fragile and do not stand up well to repeated probings. The uniformity and overall effectiveness of transfer of protein from the gel to the membrane can be checked by staining the membrane with Coomassie Brilliant Blue or Ponceau S dyes. Once transferred, proteins are detected using labeled primary antibodies, or unlabeled primary antibodies followed by indirect detection using labeled protein A or secondary labeled antibodies binding to the Fc region of the primary antibodies.

C. Immunohistochemistry

The antibodies of the present disclosure may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors, and is well known to those of skill in the art (Brown et al., 1990; Abbondanzo et al., 1990; Allred et al., 1990).

Briefly, frozen-sections may be prepared by rehydrating 50 ng of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections from the capsule. Alternatively, whole frozen tissue samples may be used for serial section cuttings.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections. Again, whole tissue samples may be substituted.

D. Immunodetection Kits

In still further embodiments, the present disclosure concerns immunodetection kits for use with the immunodetection methods described above. As the antibodies may be used to detect Human respiratory syncytial virus or Human respiratory syncytial virus antigens, the antibodies may be included in the kit. The immunodetection kits will thus comprise, in suitable container means, a first antibody that binds to Human respiratory syncytial virus or Human respiratory syncytial virus antigen, and optionally an immunodetection reagent.

In certain embodiments, the Human respiratory syncytial virus antibody may be pre-bound to a solid support, such as a column matrix and/or well of a microtitre plate. The immunodetection reagents of the kit may take any one of a variety of forms, including those detectable labels that are associated with or linked to the given antibody. Detectable labels that are associated with or attached to a secondary binding ligand are also contemplated. Exemplary secondary ligands are those secondary antibodies that have binding affinity for the first antibody.

Further suitable immunodetection reagents for use in the present kits include the two-component reagent that comprises a secondary antibody that has binding affinity for the first antibody, along with a third antibody that has binding affinity for the second antibody, the third antibody being linked to a detectable label. As noted above, a number of exemplary labels are known in the art and all such labels may be employed in connection with the present disclosure.

The kits may further comprise a suitably aliquoted composition of the Human respiratory syncytial virus or Human respiratory syncytial virus antigens, whether labeled or unlabeled, as may be used to prepare a standard curve for a detection assay. The kits may contain antibody-label conjugates either in fully conjugated form, in the form of intermediates, or as separate moieties to be conjugated by the user of the kit. The components of the kits may be packaged either in aqueous media or in lyophilized form.

The container means of the kits will generally include at least one vial, test tube, flask, bottle, syringe or other container means, into which the antibody may be placed, or preferably, suitably aliquoted. The kits of the present disclosure will also typically include a means for containing the antibody, antigen, and any other reagent containers in close confinement for commercial sale. Such containers may include injection or blow-molded plastic containers into which the desired vials are retained.

### VI. EXAMPLES

The following examples are included to demonstrate preferred embodiments. It should be appreciated by those of skill in the art that the techniques disclosed in the examples that follow represent techniques discovered by the inventors to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### Materials and Methods

Enzyme linked immunosorbent assay (ELISA) for binding to RSV F protein. For recombinant protein capture ELISA, 384-well plates were treated with 2 μg/mL of antigen for one hour at 37° C. or overnight at 4° C. Following this, plates were blocked for one hour with 2% milk supplemented with 2% goat serum. Primary mAbs and culture supernatants were applied to wells for one hour following three washes with PBS-T. Plates were washed with PBS-T four times before applying 25 μL secondary antibody (goat anti-human IgG Fc, Meridian Life Science) at a dilution of 1:4,000 in blocking solution. After a one-hour incubation, the plates were washed five times with PBS-T, and 25 μL of phosphatase substrate solution (1 mg/mL phosphatase substrate in 1 M Tris aminomethane, Sigma) was added to each well. The plates were incubated at room temperature before reading the optical density at 405 nm on a Biotek plate reader. The palivizumab competition assay ELISA was conducted by coating ELISA plates with the desired 2 μg/mL of the desired antigen. Following this, serially diluted competing mAbs spiked with 50 ng/mL biotinylated palivizumab were added to the plates. Alternatively, serially diluted serum was spiked with 50 ng/mL biotinylated palivizumab. Control wells contained PBS with 50 ng/mL biotinylated palivizumab. Palivizumab was biotinylated using the EZ-Link NHS PEG4 Biotinylation Kit (ThermoFisher) following the manufacturer's protocol. After a one hour incubation, the plates were washed with PBS-T and streptavidin-HRP (ThermoFisher) diluted 1:4000 in blocking solution was applied for one hour. After a washing step, plates were incubated with 1-step Ultra TMB solution (ThermoFisher). The reaction was stopped by adding an equal volume of 1M HCl. Plates were read on a Biotek plate reader at 450 nm.

Human hybridoma generation. Participation of healthy human adult subjects was approved by the Vanderbilt University Institutional Review Board, and blood samples were obtained only after informed consent. PBMCs were isolated from human donor blood samples using Ficoll-Histopaque density gradient centrifugation. Approximately ten million PBMCs were mixed with 17 mL of ClonaCell-HY Medium A (StemCell Technologies), 8 μg/mL of CpG (phosphorothioate-modified oligodeoxynucleotide ZOEZOEZZZZZOEEZOEZZZT (SEQ ID NO: 19), Invitrogen), 3 μg/mL of Chk2 inhibitor II (Sigma), 1 μg/mL of cyclosporine A (Sigma), and 4.5 mL of filtered supernatant from a culture of B95.8 cells (ATCC VR4492) containing Epstein-Barr virus (EBV) and plated in a 384-well plate. After seven to ten days, culture supernatants were screened for binding to recombinant, post-fusion RSV strain A2 F protein and FFL_001. Cells from positive wells were expanded into single wells in a 96-well culture plate using culture medium containing 8 μg/mL CpG, 3 μg/mL Chk2 inhibitor II, and irritated heterologous human PBMCs (Nashville Red Cross). After one week, culture supernatants were screened by ELISA for binding to recombinant, post-fusion RSV A2 F protein and FFL_001. Cells from positive wells were fused with HMMA2.5 myeloma cells by electrofusion (26). Fused cells were plated in 384-well plates in growth medium containing 100 μM hypoxanthine, 0.4 μM aminopterin, 16 μM thymidine (HAT Media Supplement, Sigma), and 7 μg/mL ouabain (Sigma). Hybridomas were screened after two weeks for mAb production by ELISA, and cells from wells with reactive supernatants were expanded to 48-well plates for one week before being screened again by ELISA, and then subjected to single-cell fluorescence-activated sorting. After cell sorting into 384-well plates containing Medium E (StemCell Technologies), hybridomas were screened by ELISA before expansion into both 48-well and 12-well plates.

Human mAb and Fab production and purification. Hybridoma cells lines were expanded in Medium E until 80% confluent in 75-cm² flasks. For antibody production, cells from one 75-cm² cell culture flask were collected with a cell scraper and expanded to four 225-cm² cell culture flasks in serum-free medium (Hybridoma-SFM, GIBCO). After 21 days, supernatants were sterile filtered using 0.45 μm pore size filter devices. For antibody purification, HiTrap MabSelectSure columns (GE Healthcare Life Sciences) were used to purify antibodies using the manufacturer's protocol. To obtain Fab fragments, papain digestion was used (Pierce Fab Preparation Kit, Thermo Scientific). Fab fragments were purified by removing IgG and Fc contaminants using a HiTrap MabSelectSure followed by purification with an anti-CH1 column (GE Healthcare Life Sciences).

Production and purification of recombinant RSV F protein RSV mAbs, and epitope immunogens. Plasmids encoding cDNAs for RSV subgroup A strain A2 or subgroup B strain 18537 pre-fusion (DsCav1) and post-fusion F protein constructs (a gift from Barney Graham) were expanded in E. coli DH5α cells and plasmids were purified using Qiagen Plasmid Maxiprep kits (Qiagen). Pre-fusion-stabilized RSV F SC-TM was synthesized (Genscript). Plasmids encoding cDNAs for the protein sequences of mAb 101F and mAb D25 were synthesized (Genscript), and heavy and light chain sequences were cloned into vectors encoding human IgG1 and lambda or kappa light chain constant regions, respectively. Mab 131-2a protein was obtained from Sigma. Commercial preparations of palivizumab (Medimmune) were obtained from the pharmacy at Vanderbilt University Medical Center. For each liter of protein expression, 1.3 mg of plasmid DNA was mixed with 2 mg of polyethylenimine in Opti-MEM I+GlutaMAX cell culture medium (Fisher). After 10 min, the DNA mixture was added to HEK293 cells at 1×10⁶ cells/mL. The culture supernatant was harvested after 6 days, and the protein was purified by HiTrap Talon crude (GE Healthcare Life Sciences) column for RSV F protein variants or HiTrap MabSelectSure columns for mAbs, following the manufacturer's protocol. 14N4Fab heavy and light variable region DNA was synthesized (Genscript) and cloned into vectors containing human CH1 and kappa sequences. 14N4Fab was expressed in Expi293 (Invitrogen) cells using Expifectamine 293 (Invitrogen) following the manufacturer's protocol. Recombinant Fab was purified using Anti-CH1 Capture Select column (GE Healthcare Life Sciences). Recombinant Fab was purified using Anti-CH1 Capture Select column (GE Healthcare Life Sciences). FFL_001, FFL_001 mutant proteins, and RPM-1 were expressed and purified as described previously (10, 25). MAb 17HD9 was expressed in expi293F cells following the manufacturer's protocol, and using the vectors described previously (Correia et al., 2014).

RSV plaque neutralization experiments. MAbs isolated from hybridoma supernatants were incubated 1:1 with a suspension of infectious RSV strain A2 for 1 hr. Following this, confluent HEp-2 cells, maintained in Opti-MEM I+GlutaMAX (Fisher) supplemented with 2% fetal bovine serum at 37° C. in a CO₂ incubator, in 24-well plates were inoculated with 50 μL of the antibody:virus or serum:virus mixture for 1 hr. After the hour, cells were overlaid with 1 mL of 0.75% methylcellulose dissolved in Opti-MEM I+GlutaMAX. Cells were incubated for four days after which the plaques were visualized by fixing cells with 10% neutral-buffered formalin and staining with crystal violet. Plaques were counted and compared to a virus control. Data were analyzed with Prism software (GraphPad) to obtain IC₅₀ values. To determine competition with 12I1, virus was first mixed with 40 μg/mL 12I1 for one hour. The virus:12I1 mixture was overlaid onto serial dilutions of 14N4 and palivizumab for one hour. The rest of the process was completed as described above.

Assays for competition-binding. After obtaining an initial baseline in kinetics buffer (ForteBio, diluted 1:10 in PBS), 10 μg/mL of his-tagged RSV F protein was immobilized onto anti-penta-his biosensor tips for a biolayer interferometry instrument (Octet Red, ForteBio) for 120 s. The baseline signal was measured again for 60 s before biosensor tips were immersed into wells containing 100 μg/mL primary antibody for 300 s. Following this, biosensors were immersed into wells containing 100 μg/mL of a second mAb for 300 s. Percent binding of a second mAbs in the presence of the first mAb was determined by comparing the maximal signal of the second mAb after the first mAb was added to the maximum signal of the second mAb alone. MAbs were considered non-competing if maximum binding of the second mAb was ≥66% of its un-competed binding. A level between 33%-66% of its un-competed binding was considered intermediate competition, and 33% was considered competing.

Antibody epitope mapping. Shotgun mutagenesis epitope mapping of anti-RSV-F antibodies was performed using an alanine scanning mutagenesis library for RSV F protein (hRSV-A2; NCBI ref # FJ614814), covering 368 surface-exposed residues identified from crystal structures of both the prefusion and postfusion conformations of RSV F. An RSV F expression construct was mutated to change each residue to an alanine (and alanine residues to serine). The resulting 368 mutant RSV F expression constructs were sequence confirmed and arrayed into a 384-well plate (one mutation per well).

Library screening was performed essentially as described previously (Davidson and Doranz, 2014). The RSV F alanine scan library clones were transfected individually into human HEK-293T cells and allowed to express for 16 hr before fixing cells in 4% paraformaldehyde (Electron Microscopy Sciences) in PBS plus calcium and magnesium. Cells were incubated with mAbs, diluted in 10% normal goat serum (NGS), for 1 hour at room temperature, followed by a 30 minute incubation with 3.75 μg/mL Alexa Fluor 488-conjugated secondary antibody (Jackson ImmunoResearch Laboratories) in 10% NGS. Cells were washed twice with PBS without calcium or magnesium and resuspended in Cellstripper (Cellgro, Manassas, Va.) plus 0.1% BSA (Sigma-Aldrich). Cellular fluorescence was detected using the Intellicyt high throughput flow cytometer (Intellicyt). Prior to library screening, to ensure that the signals were within the linear range of detection, the optimal screening concentrations for each mAb were determined using an independent immunofluorescence titration curve against cells expressing wild-type RSV F.

Antibody reactivity against each mutant protein clone was calculated relative to wild-type protein reactivity by subtracting the signal from mock-transfected controls and normalizing to the signal from wild-type protein-transfected controls. Mutations within clones were identified as critical to the mAb epitope if they did not support reactivity of the test MAb, but supported reactivity of other antibodies. This counter-screen strategy facilitates the exclusion of RSV F protein mutants that are misfolded or have an expression defect. The detailed algorithms used to interpret shotgun mutagenesis data are described elsewhere (Davidson and Doranz, 2014).

Crystallization and structure determination of 14N4-Fab and 14N4-Fab-RSV F. Recombinant 14N4-Fab was concentrated to 10 mg/mL and a crystal was obtained in Hampton Index HT screen condition 20% PEG 3350, 50 mM zinc acetate. The crystal was harvested directly from the screening tray, cryoprotected in the mother liquor with 20% glycerol, and data was collected using a Bruker Microstar microfocus rotating-anode X-ray generator equipped with a Bruker Proteum PT135 CCD area detector, and Proteium2 software (Bruker-AXS). Data was processed with XPREP (Sheldrick, 2007) to 2.0 Å. The structure of 14N4-Fab were determined by molecular replacement in Phaser (Adams et al., 2010) using the separate constant and variable domain models from PDB 4Q9Q. The model was improved through iterative refinements in Phenix (Adams et al., 2010) and manual building in Coot (Emsley and Cowtan, 2004), guided by composite omit maps.

To crystallize 14N4 in complex with RSV F, both hybridoma-cleaved 14N4 and RSV A2 F were buffer-exchanged in excess into 50 mM Tris pH 7.5, 50 mM NaCl. 14N4-Fab was mixed in excess with RSV A2 F post-fusion protein and incubated at 37° C. for two hours. Following this, the sample was subjected to size exclusion chromatography (S200, 16/300, GE Healthcare Life Sciences) in 50 mM Tris pH 7.5, 50 mM NaCl. The complex was concentrated to 10 mg/mL and crystals were obtained in Hampton Crystal Screen HT in 2 M ammonium sulfate, 5% 2-propanol. Approximately forty crystals were screened for diffraction, and numerous cryoprotectants were tried, however, the best diffraction obtained was to 4.1 A using the mother liquor with 20% glycerol as a cryoprotectant. X-ray diffraction data were collected at the Advanced Photon Source LS-CAT beamline 21-ID-F. Data were indexed and scaled using XDS (Kabsch, 2010). A molecular replacement solution was obtained in Phaser (Adams et al., 2010) using RSV A2 F protein trimer PDB 3RRR and the structure of 14N4-Fv region. Significant density, albeit shifted from the apo-structure, was observed for the constant region, and a solution could be obtained in Phaser with the constant region. The structure was refined using Group B-factors, coordinates, NCS restraints, and 14N4-Fab and PDB 3RRR as reference models restraints. The density around the 14N4-RSV F interface was well defined and CDR loops matched well with the apo-14N4 structure. Data collection and refinement statistics are shown in Table S1.

Negative-stain electron microscopy. 14N4-Fab was mixed in excess with RSV 18537 B post-fusion F protein and incubated at 37° C. for one hour. Following this, the complex was purified by size exclusion chromatography (S200, 16/300, GE Healthcare Life Sciences) in 50 mM Tris pH 7.5, 50 mM NaCl. Carbon-coated copper grids were overlaid with the complex at 5 μg/mL for three minutes. The sample was washed in water twice and then stained with 0.75% uranyl formate for one minute. Negative stain micrographs were acquired using an FEI Tecnai F-20 transmission EM scope and a Gatan 4k×4k CCD camera using 50,000× magnification at a defocus of −1.5 μm. Micrographs were rescaled by a factor of two resulting in a final image with 4.36 Å/px. Particles were picked manually using EMAN Boxer (Tang et al., 2007) with a box size of 75 pixels and pixel size of 5.25 nm/px. Reference-free 2D classification was performed using Spider (Shaikh et al., 2008).

Surface plasmon resonance. Binding experiments using surface plasmon resonance were carried out on a ProteON XPR36 instrument (Bio-Rad). For this experiment, the inventors used GLC sensor chips (Bio-Rad). To determine detection of Fab binding, FFL_001 was captured using the anti-his mab (Immunology Consultants Laboratory, Clone 7B8). Mutated FFL_001 (R33C, N72Y, K82E) was used as a binding control. Fabs were injected as analytes in running buffer HBSEP+ (Teknova) with 1 mg/ml BSA at a flow rate of 50 μl/min. The surface was regenerated with 0.85% phosphoric acid (Bio-Rad), 4 injections, 15 seconds contact time each. The inventors analyzed data using Proteon Manager software (Bio-Rad, version 3.1.0.6). Binding responses were double referenced against inter-spot and reference channel. They fit the data with Simple Binding Langmuir model.

Hydrogen-deuterium exchange mass spectrometry. Deuterium exchange was initiated by addition of 6.6 μL 14N4 Fab (2.0 mg/mL) and 3.3 μL either scaffold (1.1 mg/mL) or water into 40 μL exchange buffer (100 mM NaCl, 20 mM Tris-HCl, pH 7.5) made in D₂O. For a nondeuterated control the reaction was performed in the same buffer made in water. The reaction was allowed to proceed for 15, 30, or 60 minutes, and was quenched by addition of 50 μL quenching buffer (0.2% formic acid, 200 mM TCEP, 4 M urea, pH 2.45). The reaction was placed on ice, and 6.6 μL of porcine gastric pepsin (20 mg/mL) (Sigma-Aldrich) was added. Protease digestion was allowed to proceed for 5 minutes on ice, after which 100 L was used for HPLC separation and mass spectrometric analysis. Each time point was performed in triplicate and the results averaged for analysis. The individual peptides were separated and analyzed for deuterium incorporation using a Rheodyne 7010 manual injector (Sigma-Aldrich) connected to a ThermoFinnigan Surveyor HPLC. Peptides were separated using Phenomenex 50×2.1 mm C18 reverse-phase column at 100 μL/min. Separation was performed using a 5-65% acetonitrile/H₂O gradient over 25 min, with 0.1% formic acid added to each buffer. The sample loop and column, as well as the chromatographic buffers, were completely submerged in an ice-water slurry to prevent excessive back exchange of deuterium atoms into the solvent. Mass spectra were recorded using a ThermoFinnigan LTQ XL ion trap mass spectrometer using positive ion electrospray ionization (ESI). The mass spectrometer was set to scan in the m/z range of 300-2,000, with the first 2 minutes of elution diverted to waste to eliminate early-eluting salts. For deuterium exchange experiments, data were collected in MS1 mode. For peptide identification the same chromatography gradient was used, with the mass spectrometer run in data-dependent mode collecting seven scan events using collusion-induced dissociation fragmentation with a collision energy of 25

V. Peptide identification was done using PEAKS software (Version 7.0, Bioinformatics Solutions Inc.). Peptides were searched using a parent mass error tolerance of 0.5 Da and a fragment mass error tolerance of 0.5 Da, using non-specific enzymatic cleavage and a charge state of 1-4. Post-translation modifications of methionine oxidation and asparagine/glutamine deamidation were considered in peptide identification. Peptides were matched against a database consisting of 14N4 heavy and light chains, as well as porcine pepsin. Only peptides with a −10 log P score of 35.3 or better were selected for deuterium exchange analysis, corresponding to a 0.05 false discovery rate (FDR). Out of all peptides identified, 15 with consistent signal and optimal coverage of all CDR loops were selected for deuterium exchange analysis. The centroid mass of each peptide was calculated for each time point and compared to the non-deuterated control to calculate the extent of deuterium incorporation. The shift in mass compared to non-deuterated control was normalized by the theoretical upper limit of deuteration for each peptide to obtain the percent deuteration. Deuterium incorporation for an individual residue was calculated as a weighted average of all fragments containing the residue, weighted by the inverse of the peptide length. This normalization strategy has been used successfully to convert deuterium exchange values to a per-residue basis for structural visualization (Sevy et al., 2013).

### Example 2

### Results

Antibody isolation, binding, and neutralization. The inventors isolated 9 human mAbs from four human donors targeting the RSV F protein using human hybridoma technology (Smith and Crowe, 2015). Transformed B cells generated from the B cells of adult human donors were screened by enzyme-linked immunosorbent assay (ELISA) for reactivity to the RSV A2 F protein. Reactive cells were fused with myelomas to create hybridoma cell lines and plated in a 384-well plate. After seven to ten days, culture supernatants were screened for binding to recombinant, post-fusion RSV A2. F protein. Cells from positive wells were expanded respectively into single wells in a 96-well culture plate using culture medium containing CpG, Chk2 inhibitor IF and irradiated heterologous human PBMCs. After one week, culture supernatants were screened by ELISA for binding to recombinant, post-fusion RSV A2 F protein. Clonal hybridomas were obtained by single-cell flow cytometric sorting, and isotyping analysis of purified mAbs showed them to be primarily of the IgG₁ subclass (Table 5).

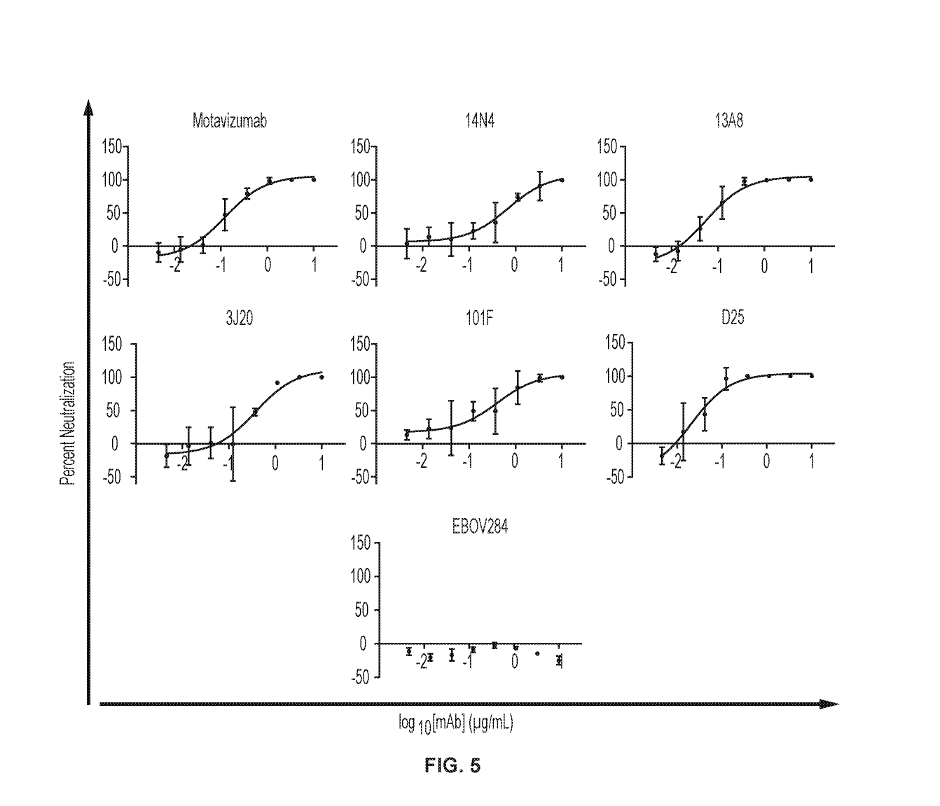

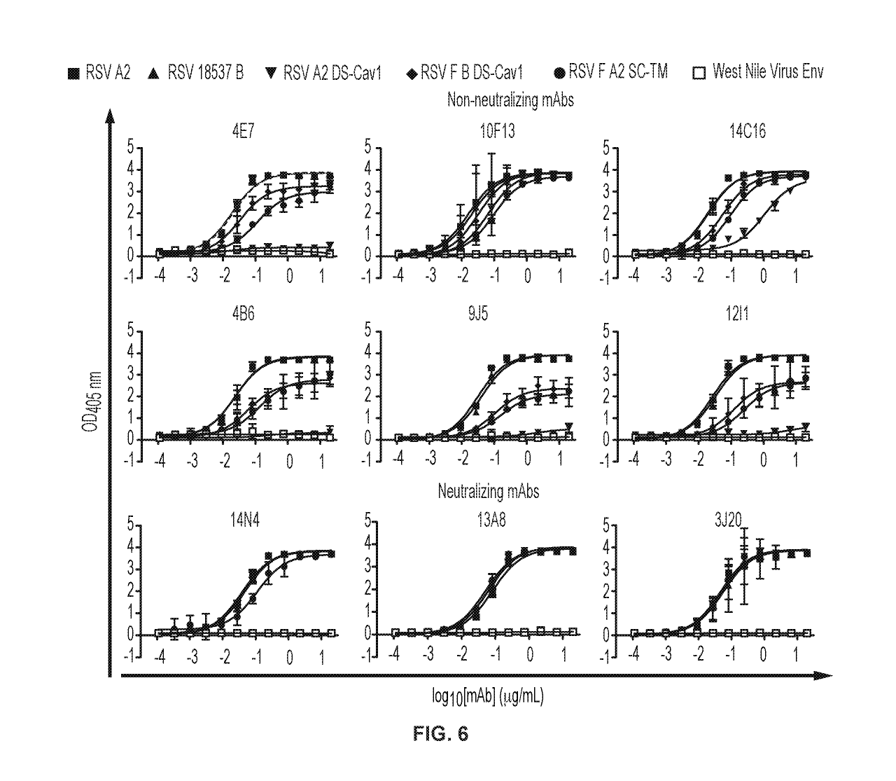

To assess whether the mAbs possessed neutralizing activity, purified mAbs were tested by plaque reduction neutralization assay using RSV strain A2. As expected, serum from two donors neutralized RSV (FIG. 5). Of the mAbs isolated, 14N4, 13A8, and 3J20 neutralized virus, while the remaining mAbs failed to show neutralization activity when tested at concentrations up to 100 μg/mL. These three neutralizing mAbs had IC₅₀ values less than 1 μg/mL (Table 5, FIG. 5). Recombinantly expressed site II mAb motavizumab (Wu et al., 2007b), and previously described mAbs to site IV (101F) (Wu et al., 2007a) and site Ø (D25) (McLellan et al., 2013a) also were tested for comparison. Mab 13A8 possessed potency similar to that of motavizumab and D25. MAbs were tested for binding by ELISA to post-fusion or pre-fusion-stabilized (Ds-Cav1 or SC-TM) RSV strain A2 F proteins (McLellan et al., 2013b; Krarup et al., 2015) and post-fusion F from RSV strain 18537 B (Table 5, FIG. 6). Determination of EC₅₀ values revealed that the three neutralizing mAbs bound to both pre-fusion and post-fusion F proteins with equal affinity, agreeing with the conservation of the antigenic site II epitope between pre- and post-fusion RSV F (Table 5, FIG. 6). Furthermore, the inventors did not detect major differences between binding to purified DS-Cav1 or SC-TM pre-fusion-stabilized F protein variants, suggesting the conformation of these antigens is similar at site II. Although the remaining mAbs did not neutralize RSV, EC₅₀ values for binding in ELISA to post-fusion F protein were similar for the neutralizing and non-neutralizing mAbs. These data suggest that the binding location or pose, rather than the affinity, is the critical determinant for RSV neutralization in this set of mAbs. MAbs 4E7, 4B6, 9J5, and 12I1 favored the post-fusion conformation, based on differences in binding to stabilized pre-fusion versus post-fusion F protein. Serum from two donors was als tested for binding, and no siginificant differences were observed among the two (FIG. 6).

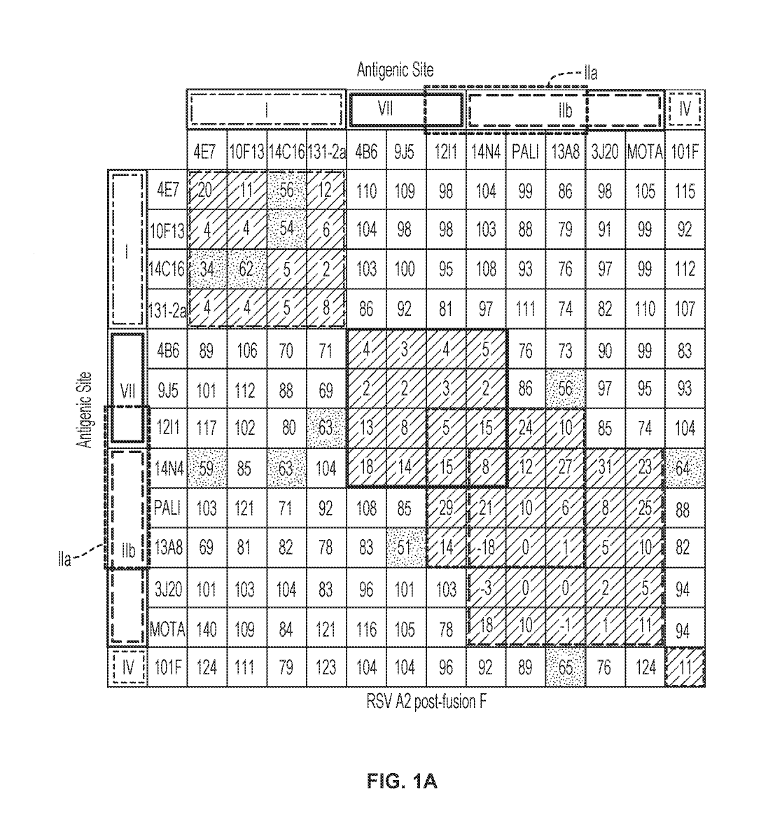

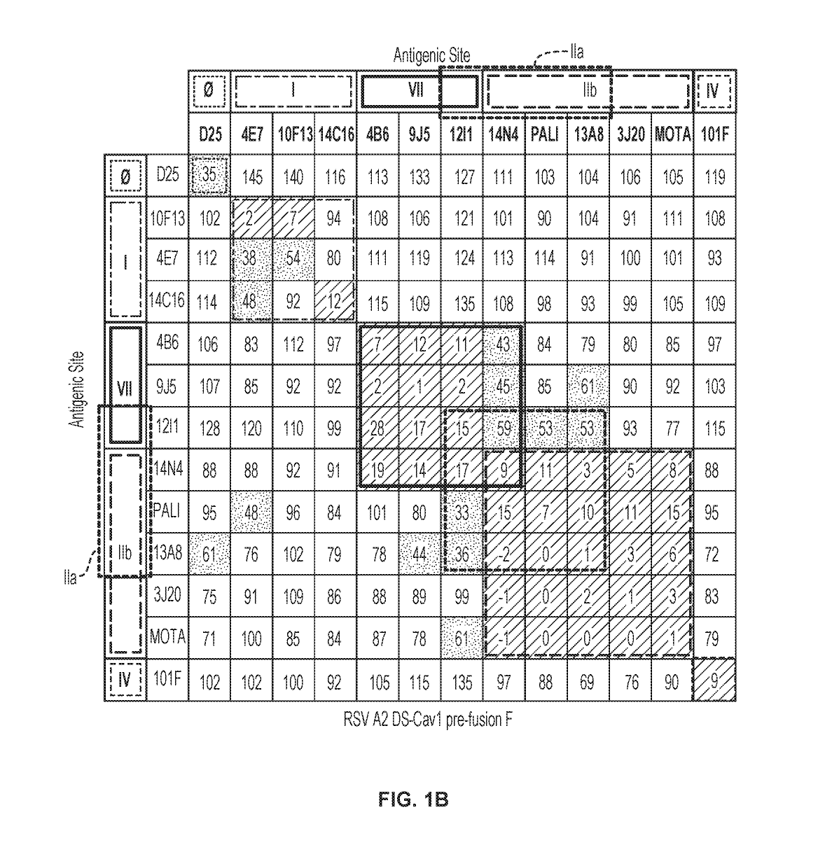

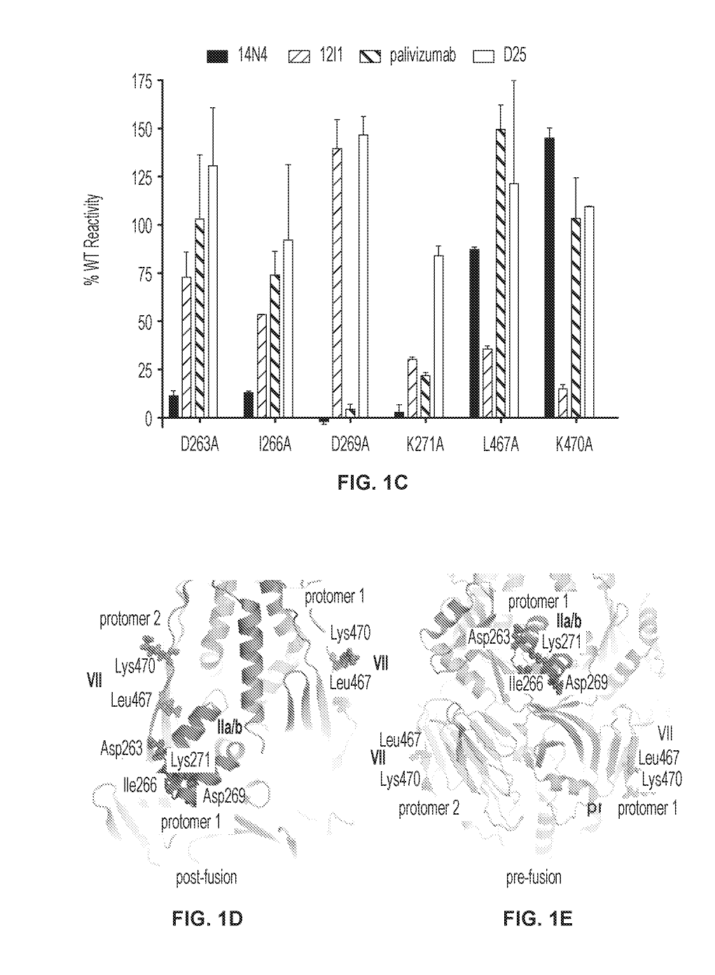

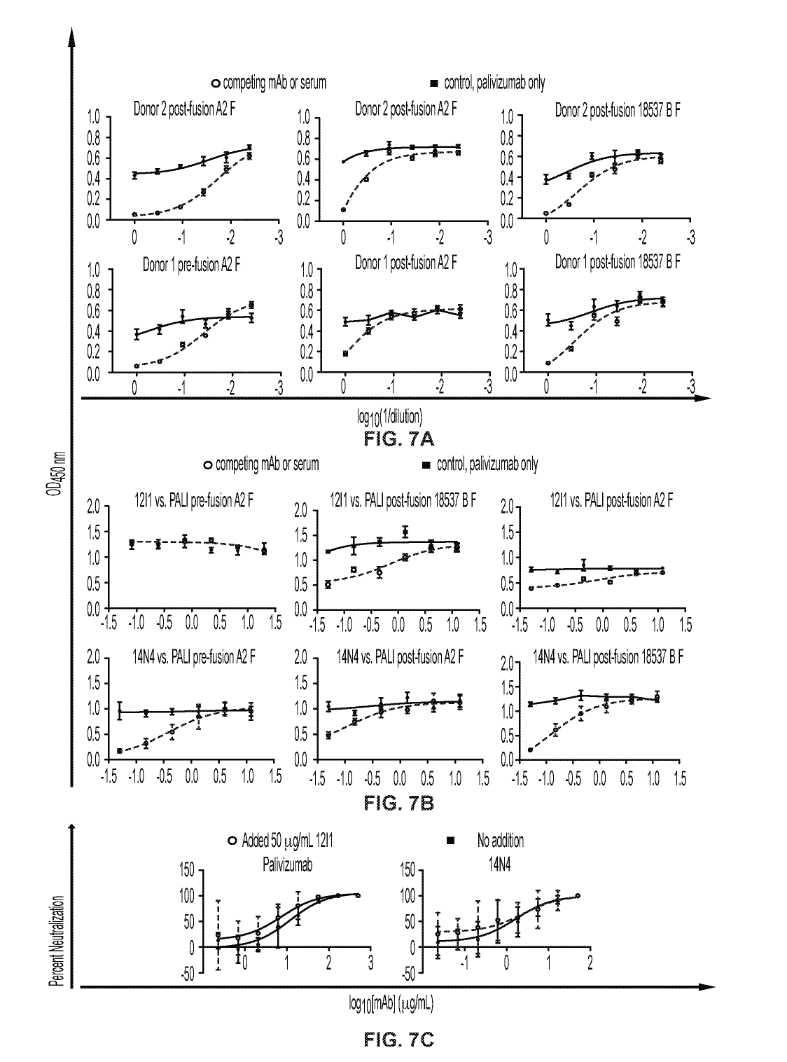

Epitope binning reveals the complexity of site II. In order to determine putative binding sites for the isolated mAbs, real-time competition-binding studies were conducted with his-tagged RSV F proteins coupled to anti-penta-his biosensor tips. The inventors included recombinant forms of the previously described RSV mAbs 101F (site IV), 131-2a (site I) (Anderson et al., 1985), palivizumab (site II), and motavizumab (site II) for comparative purposes in the competition-binding study on post-fusion and pre-fusion F, since the epitopes for those mAbs have been defined previously. A complex array of five distinct competition-binding groups was observed for binding to post-fusion F (FIG. 1A). The groups containing mAbs binding to antigenic sites I, II, and IV were identified using the control mAbs. Three mAbs targeted site I, a neutralizing epitope present near the membrane proximal region of the F protein. However, none of these mAbs possessed neutralizing activity. The previously reported murine mAb 131-2a exhibits a low level of neutralizing activity (McLellan et al., 20013a), but recognition of this epitope by human mAbs was not associated with neutralization, suggesting antigenic site I is not a major target of the human neutralizing antibody response. The remaining mAbs competed with antibodies directed to antigenic site II. Three mAbs (4B6, 9J5, 12I1) competed with site II-specific antibodies, yet failed to neutralize RSV, suggesting they do not bind in the correct orientation or they do not contact the full complement of critical amino acid residues in the site. The three neutralizing mAbs 14N4, 13A8, and 3J20 competed for binding to post-fusion F with both palivizumab and motavizumab, as would be expected for mAbs targeting antigenic site II, yet subtle differences were observed among the competition patterns. MAb 3J20 differed from the other two by competing only with other neutralizing mAbs. The most potent mAb, 13A8, showed approximately 50% competition with the non-neutralizing mAb 9J5 and directly competed with 12I1. Interestingly, mAb 14N4 directly competed with all three non-neutralizing mAbs, forming a block of four mAbs containing both neutralizing and non-neutralizing mAbs. Furthermore, intermediate one-directional competition was observed for 14N4 with site I mAbs 4E7 and 14C16. Based on these data, it is apparent that mAbs competing for antigenic site II constitute at least three groups, which the inventors designated antigenic sites IIa and IIb for neutralizing poses, and site VII for the non-neutralizing site. Antigenic site VII is represented by the non-neutralizing mAb 12I1. Antigenic site IIb, containing mAb 3J20 and motavizumab, is a discrete competition group containing only neutralizing mAbs. Antigenic site IIa is an intermediate site, distinguished from site IIb as competing with both neutralizing and non-neutralizing mAbs, and is recognized by mAbs 14N4, 13A8, and palivizumab. Further differences in competition patterns within the site IIa group of mAbs were observed, as 14N4 competes with all three non-neutralizing mAbs, 13A8 competes with two, and palivizumab competes with one, suggesting a gradient of binding poses occur at antigenic site IIa between sites VII and IIb. The inventors also tested competition using pre-fusion F (DS-Cav1) as the immobilized antigen, and included the pre-fusion-specific mAb D25 for comparison (FIG. 1B). Although site VII mAbs do not bind well to pre-fusion F protein by ELISA, the inventors observed significant binding in biolayer interferometry experiments, allowing competition studies to be conducted with pre-fusion F. A similar pattern of three distinct groups was observed for antigenic site II in pre-fusion F, however competition at site IIa was weaker among mAbs in the group, suggesting sites VII and IIa may be further apart in the pre-fusion than in the post-fusion conformation. Such a complex array of competition-binding groups was unexpected, since the site II mAb palivizumab, which is used in prophylactic treatment, also bi-directionally competed with the non-neutralizing mAb 12I1. A palivizumab-competition assay designed to detect the presence of site II antibodies in immune serum by competing with palivizumab (Smith et al., 2012; Raghunandan et al., 2014) has been proposed as a correlate of immunity for an RSV post-fusion F protein vaccine candidate. Indeed, the inventors repeated the competition using published palivizumab competition assay protocols (Smith et al., 2012) where biotinylated palivizumab was spiked into control mAbs, as well as donor serum. As expected, tjeu observed donor serum neutralized RSV and competed with palivizumab at low dilutions (FIGS. 7A-C). Furthermore, mAbs 14N4 and 12I1 both competed with palivizumab, with 12I1 showing competition only on post-fusion F, similar to the competition data in FIGS. 1A-E. Based on the data described, it appears motavizumab and 3J20-like mAbs may be better candidates for this purpose, as competition with these mAbs is observed only with neutralizing mAbs, but the palivizumab-competing antibody population contains a proportion of non-neutralizing mAbs. To determine if the non-neutralizing mAb 12I1 blocked neutralization of palivizumab or 14N4, the inventors incubated mAb 12I1 with virus initially before applying the neutralizing mAbs. No significant difference was observed between those samples incubated with 12I1 and control mAbs (FIGS. 7A-C). This finding is expected as 12I1 favors the post-fusion conformation (Table 5), which allows membrane fusion by the F protein before 12I1 binding. Thus, the site VII mAbs do not inhibit neutralization, yet are likely produced in response to a post-fusion F immunogen, and also affect the palivizumab competition assay.

Saturation alanine scanning mutagenesis. To better understand the complexity of antigenic site II and the specificity of mAbs recognizing the site, the inventors performed saturation alanine scanning mutagenesis to identify residues critical for the binding of the neutralizing mAb 14N4 or non-neutralizing mAb 12I1. Residues Asp263, Ile266, Asp269, and Lys271 were critical for 14N4 binding (FIG. 1C). Interestingly, the inventors previously identified a Ile266Met mutation when generating monoclonal antibody-resistant mutant (MARM) virus by in vitro selection using the RSV F targeting human Fab19 (Crowe et al., 1998a), isolated from a phage display library. Based on the X-ray crystal structure of the RSV F protein (FIG. 1D), Ile266 is positioned at the bottom of the antigenic site II helix-loop-helix motif and is pointed toward the inner protein core, suggesting the residue disrupts the antigenic motif by allosteric effects. In the same study (Crowe et al., 1998a), selection with several murine mAbs produced MARM viruses with Lys272Asn, and similarly, selection with palivizumab in vitro or in vivo generated similar MARM viruses with the following mutations: Lys272Met, Lys272Gln, as well as Asn2681Ile (Zhao et al., 2004a; Zhao and Sullender, 2005). The Lys272Gln MARM virus completely resisted prophylactic palivizumab treatment (Zhao et al., 2004b). Unexpectedly, mutagenesis scanning for the site VII mAb 12I1 revealed critical residues over 40 Å away in the RSV F monomer: Leu467 and Lys470 (FIGS. 1C-D). While the site VII mAb 12I1 and site IIa mAb 14N4 competed for binding, the critical residues for binding were quite different, with site VII residues falling on the 47 Å extended loop connecting the lower structured portion to the helix bundle in a single protomer of F in post-fusion conformation (FIG. 1D). However, when the F protein is viewed as a trimeric structure, all residues in antigenic sites VII and IIa come in close proximity through quaternary interactions. Antigenic site IIa in one protomer of F in the trimer is within 13 Å of antigenic site VII on an adjacent protomer. While a quaternary epitope for RSV F has been described for the mAb AM14 (Gilman et al., 2015), the site VII/IIa mAb competition is the first described example of quaternary interactions contributing to non-neutralizing mAb competition with a neutralizing mAb. In the pre-fusion conformation (FIG. 1E), antigenic sites VII and IIa are farther apart than in the post-fusion form. Antigenic site IIa is equidistant from site VII on the same and the adjacent protomer. This difference confirms the observation in the epitope binning studies in which competition on pre-fusion F between antigenic sites IIa and VII was less pronounced than in the post-fusion conformation. The intermediate level of competition for binding to the pre-fusion form of F between sites VII and IIa mAbs was consistent for mAbs 14N4, 13A8, and palivizumab.

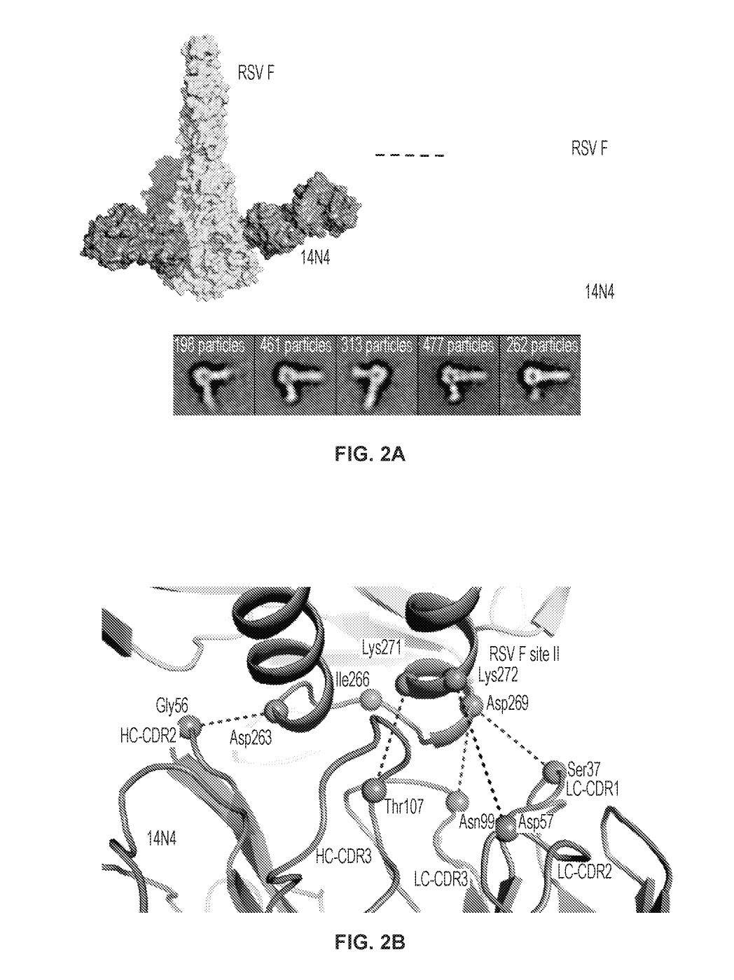

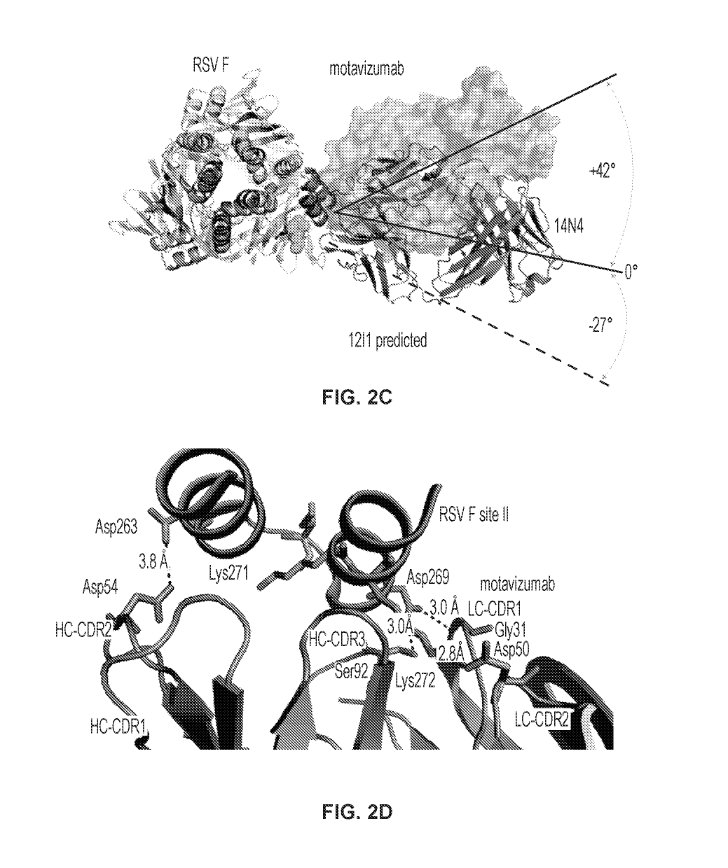

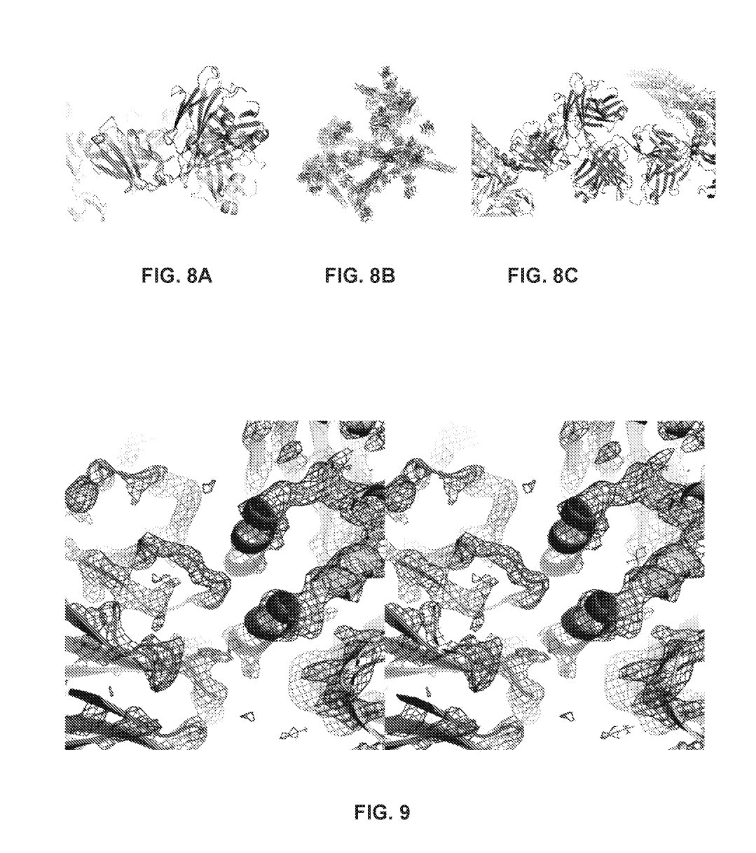

Structure of the 14N4-Fab-RSV F complex. Since 14N4 is a unique mAb, competing not only with palivizumab, but also with non-neutralizing mAbs, the inventors next sought to determine the structural basis for competition of 14N4 with other mAbs recognizing site II. The 14N4 fragment antigen-binding region (14N4-Fab) was crystallized in spacegroup P 1 2₁ 1 and the structure was solved to 2.0 Å with R_{work}/R_{free}=19.5/21.0% (Table S1). 14N4-Fab then was incubated with post-fusion RSV A2 F, and the complex was isolated by size exclusion chromatography and crystallized in spacegroup P 4₂ 2₁ 2. After screening with numerous cryoprotectants and attempts at data collection at room temperature, the best X-ray diffraction of the complex was to 4.1 Å (Table S1). The crystal structures of post-fusion RSV F and 14N4 variable and constant Fab regions were used in molecular replacement to solve the structure of the complex with R_{work}/R_{free}=25.6/28.2%, refined using NCS torsion and reference-model restraints. Separate searches were needed for the variable and constant regions of the 14N4-Fab region as the constant region was shifted 56° from the apo-14N4-Fab structure, an observation likely attributed to crystal packing, as the constant region makes contacts to the next asymmetric unit (FIGS. 8A-C). The asymmetric unit is composed of the RSV F trimer with three 14N4-Fab molecules, one at each protomer of RSV F (FIG. 2A). Electron density for the RSV F protein and the three 14N4-Fab variable regions was well defined, especially at each interface between the two molecules (FIG. 9). To confirm binding at antigenic site II in RSV strain 18537 B, the inventors complexed 14N4 with RSV 18537 B post-fusion F and class-averages determined from negative-stain electron microscopy images indicated the position and orientation of the 14N4-Fab molecules were similar to those in the X-ray crystal structure (FIG. 2A). The HCDR3 of 14N4-Fab nestles between the two helices in the antigenic site II motif, where several hydrophobic residues exist. Residues in the RSV F structure important for binding based on alanine scanning mutagenesis are highlighted in FIG. 2B, where they make key interactions with 14N4-Fab. Asp263 is within hydrogen bonding distance of the backbone Gly56 on 14N4, and Lys271 likely interacts with the heavy chain CDR3 by hydrogen bonding with Thr107 (FIG. 2B). Furthermore, the light chain also appears important for binding, since Asn99 and Ser37 of the light chain CDR1 are in close contact with Asp269. Lys272 is near of the light chain CDR2 Asp57, although this residue was not critical for binding in mutagenesis scanning experiments. As expected, interactions were not observed for Ile266, as this residue is buried at the base of the helix-loop-helix motif. When compared to the structure of motavizumab in complex with the site II peptide, striking differences were observed. Overlaying at antigenic site II, the motavizumab angle of binding is significantly different, as it is shifted 42° from the 14N4 binding region in the direction away from the 12I1 site VII (FIG. 2C). This structural difference correlates with the lack of competition between antigenic site IIb mAbs motavizumab and 3J20, and the antigenic site VII non-neutralizing mAbs binding at Leu467 and Lys470. 14N4 could indeed block the binding of 12I1, since its binding pose is predicted to be shifted just 27° from site VII. Yet motavizumab is shifted away from site IIa enough to prevent competition with mAb 12I1. Considering critical binding interactions, the inventors noted that motavizumab hydrogen bonds to Asp263 using Asp54 (HCDR2) distantly, to Lys272 with Asp50 (LCDR2), and Asp269 using Ser92 (LCDR3) (FIG. 2D). Interestingly, motavizumab bypasses Lys271, leaving no residues in the vicinity with which to interact. This positioning causes a shift away from site VII, as the majority of the interactions are involved on the right helix, rather than the left helix, where only hydrophobic interactions exist with the motavizumab HCDR3.

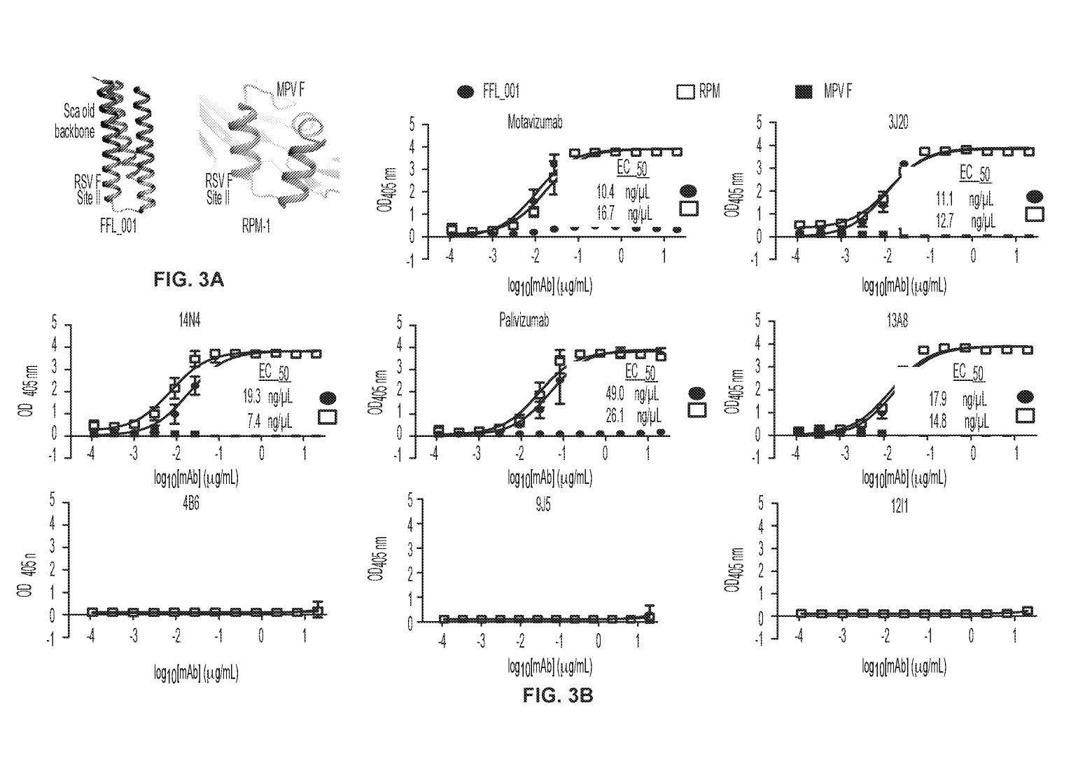

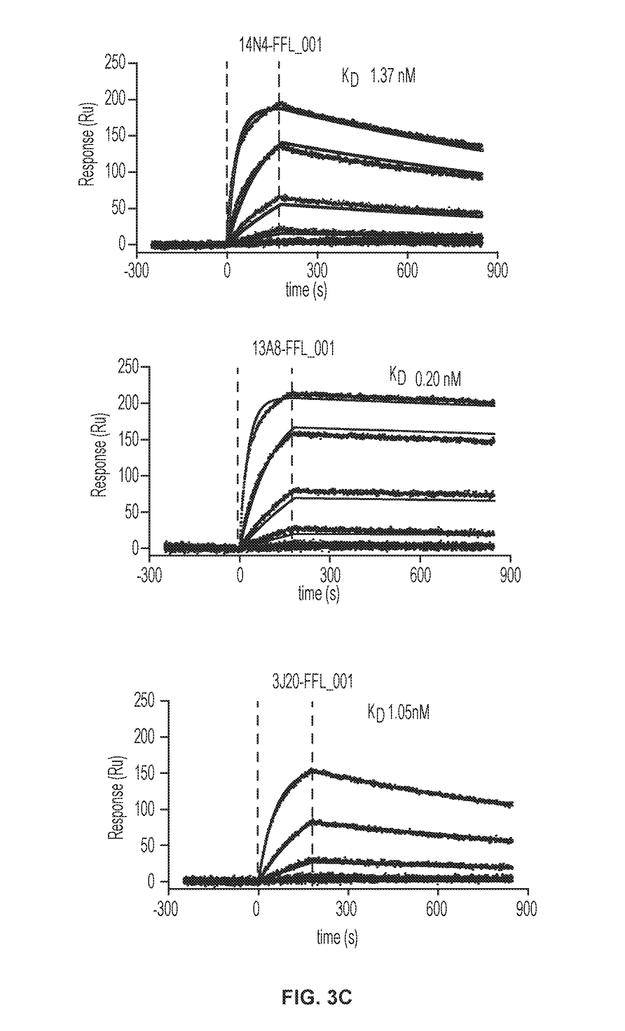

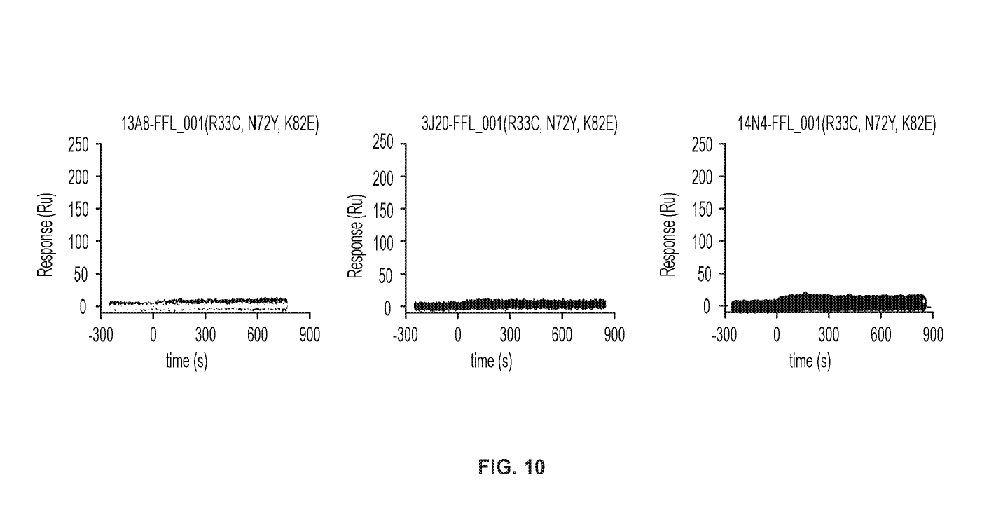

Human antibodies bind scaffold-based immunogens. Attempts to generate a vaccine against RSV have been largely unsuccessful, and the presence of non-neutralizing mAbs competing with neutralizing mAbs may contribute to this problem. The inventor and others have recently reported structure-based designed vaccine candidates for presenting the site II immunogen. Strategies included an epitope helix-loop-helix motif of antigenic site II grafted onto a stable tri-helix scaffold protein (FFL_001) (Correia et al., 2014), an immunoglobulin-based scaffold for site II (Luo et al., 2015), and also a strategy in which the RSV site II was grafted onto the metapneumovirus F protein (RPM-1) in order to generate a chimeric protein capable of inducing a cross-reactive immunogenic response (Wen et al., 2016) (FIG. 3A). Each of these three epitope-based scaffolds induced at least partial immune responses in mice to RSV F, and the FFL_001 vaccine candidate induced reasonable titers of neutralizing mAbs from immunized macaques. The inventors tested binding by ELISA of the three neutralizing site II human mAbs 14N4, 13A8, and 3J20 to FFL_001 and RPM-1 and found that they did bind, as did palivizumab and motavizumab used as positive controls (FIG. 3B). EC₅₀ values for binding of the mAbs to the scaffolded epitopes were similar to those obtained for the RSV F protein, suggesting antigenic site II is the primary region necessary for human mAb binding. This finding also is consistent with the X-ray crystallography and EM structural data for the 14N4-Fab-RSV F complex. Interestingly, binding was not detected for the non-neutralizing mAb 12I1 or other antigenic site VII mAbs to either FFL_001 or RPM-1 scaffold proteins. Therefore, binding to the scaffolded epitopes distinguishes neutralizing from non-neutralizing site VII competing antibodies. Surface plasmon resonance revealed very low K_{D} values for the three neutralizing mAbs (FIG. 3C) suggesting limited residues are needed for Fab binding to antigenic site II, a finding consistent with the X-ray structure of 14N4-Fab with RSV F, as no molecular contacts were observed outside site II. However, additional interacting residues may be present in 14N4 binding to pre-fusion RSV F. Binding was not detected to a mutated FFL_001 control (FIG. 10).

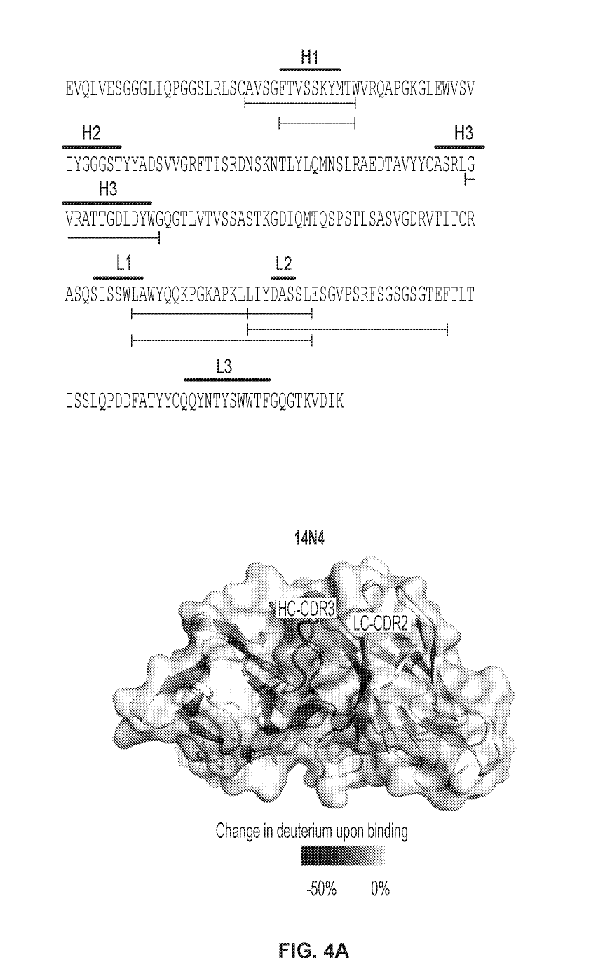

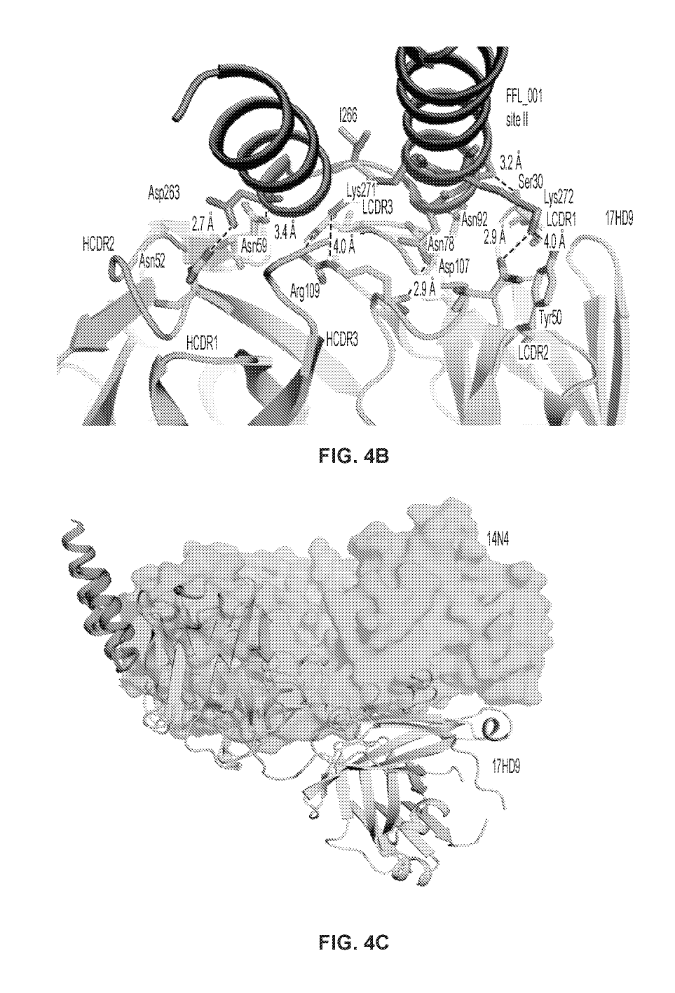

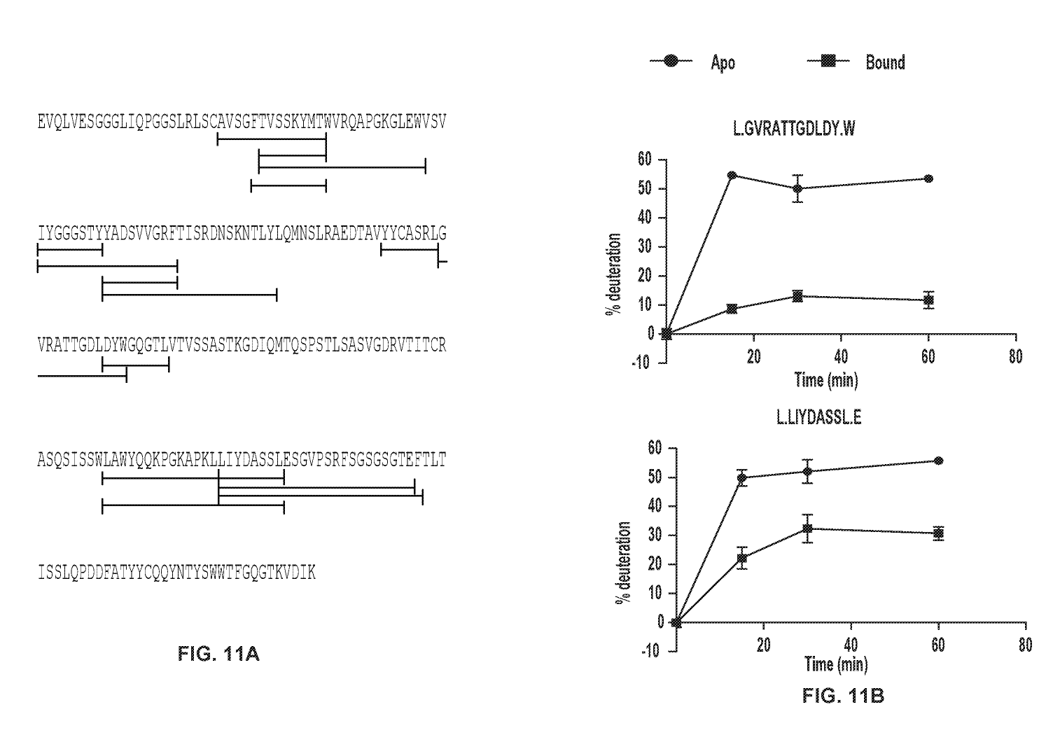

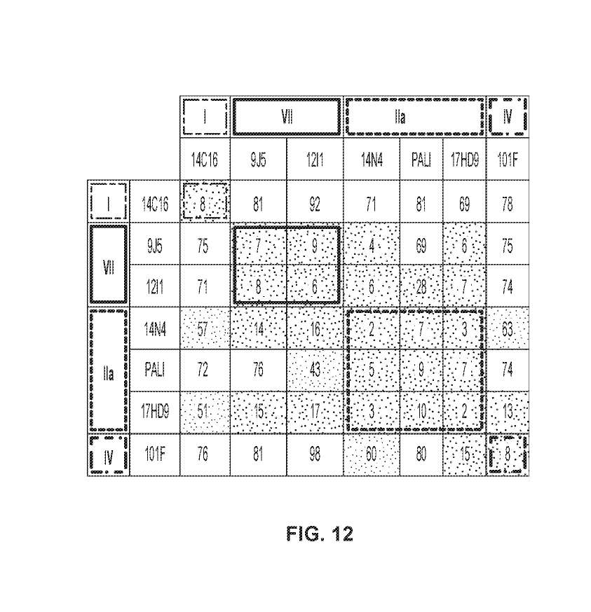

In order to confirm the binding location for 14N4 to the FFL_001 scaffolded epitope, the performed hydrogen-deuterium exchange mass spectrometry (FIG. 4A). The inventors mapped the majority of the 14N4-Fab region (FIGS. 11A-B), and the peptides with the largest decrease in deuterium exchange in the bound state were localized to the HCDR3 loop, with a limited effect in the LCDR2. This finding is largely consistent with the crystal structure of 14N4-Fab with RSV F, as the HCDR3 is buried in the antigenic site II motif, and the LCDR2 makes interactions through Asp57. These data suggest 14N4 binds the scaffolded epitope using similar residues as with RSV F. Indeed, significant differences were not observed between X-ray structures of motavizumab in complex with FFL_001 and motavizumab in complex with the antigenic site II peptide (Correia et al., 2014), further suggesting the scaffold-based approach allows similar binding poses. The inventors also compared the binding poses of the neutralizing macaque mAb 17HD9, isolated following FFL_001 immunization, and crystallized in complex with FFL_001 (Correia et al., 2014). MAb 17HD9 has an extended HCDR3 compared to 14N4 and motavizumab, and is positioned horizontally across the antigenic site II motif, unlike 14N4, where the HCDR3 is positioned vertically, inserting itself between the two helices (FIG. 4B). The extended CDR3 residues Arg109 and Asp107 make contacts with Lys271 and Lys272. Furthermore, the LC-CDR loops are positioned to make key contacts with the bottom of helix 2, a feature that allows mAb 17HD9 to interact with antigenic site II at a different angle, where the Fab is shifted downward as compared to 14N4 and motavizumab (FIG. 4C). MAb 17HD9 is positioned further left than 14N4, close to antigenic site VII, suggesting that 17HD9 would compete with 12I1 and other site VII mAbs. Indeed, the inventors observed such competition between recombinantly expressed mAb 17HD9 and site VII mAbs (FIG. 12).

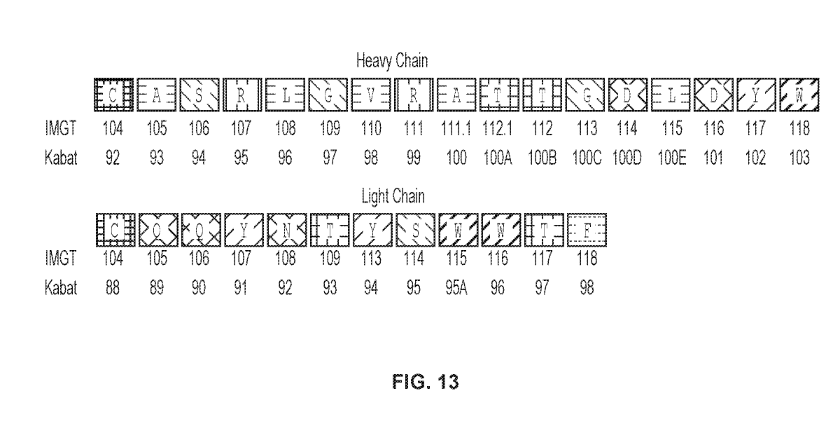

MAb 14N4 uses V_{H}3-53 and J_{H}4 gene segments to encode the expressed antibody (HCDR3 numbering in FIG. 13). Because of the paucity of human antibodies that target RSV antigenic site II, it was unclear if this mAb is unique among human donors, or if 14N4-like mAbs exist that do compete with non-neutralizing mAbs in the general population. To help answer this question, the inventors searched a database of 50 million antibody heavy chain variable sequences obtained from 31 adult human subjects, and found similar sequences in 31 individuals that used V_{H}3-53 and J_{H}4 gene segments and shared 85% similarity in the HCDR3 (Table 6). When the HCDR3 identity cutoff for matching was extended to 100%, the majority of sequence matches remained. These sequence homology data suggest that 14N4-like mAbs may be common in the human population, and the presence of non-neutralizing mAbs competing with neutralizing mAbs may be a common feature in human RSV immune responses.

### Example 3

### Discussion

Although palivizumab has been used as a prophylactic treatment for high-risk infants during RSV season for nearly two decades, no vaccine is currently approved for protection against RSV. Vaccine strategies have been proposed that focus on the 150 kDa post-fusion RSV F trimeric protein to elicit an immune response, yet antibody production is directed toward both protective and non-protective epitopes. The inventors have shown in the newly described human mAbs evidence for substantial neutralizing/non-neutralizing mAb competition binding at antigenic site II. Considering the competition patterns, antigenic site II was delineated into two sub-sites based on epitopes on adjacent protomers of the RSV F trimer, and a new region, site VII, was characterized as a non-neutralizing antigenic site that competes with site II. Based on the X-ray structure of 14N4 in complex with RSV F, subtle changes in the binding pose can cause substantial effects in competing antibodies. While the competition was described here for RSV, these data may inform general vaccine design, as non-neutralizing antibody production is a common occurrence during viral infection. Furthermore, studying the B cell response of vaccinated individuals in clinical trials will assist in determining the extent of neutralizing/non-neutralizing mAb competition in human sera.

Competition between 14N4 and 12I1 mAbs on post-fusion F is readily observed, as the 12I1 site VII is in close proximity to antigenic site IIa. However, the competition was less pronounced in the pre-fusion conformation, as sites VII and IIa are not in close proximity before the pre- to post-fusion rearrangement. As 12I1 favors the post-fusion conformation (Table 5), vaccine strategies involving pre-fusion F may be more beneficial to avoiding the competing interactions at antigenic site II. Indeed, 12I1 was likely generated against the RSV F post-fusion conformation, and these 12I1-like mAbs may not have been isolated if prefusion F was used in the initial B cell isolation. Future experiments detailing the mAb response to pre-fusion F will be beneficial in determining the overall impact of the competition with non-neutralizing mAbs. When assessing vaccine efficacy using competition with palivizumab, non-neutralizing antibody competition with palivizumab must be taken into account, especially in vaccine candidates utilizing post-fusion RSV F. The inventors further propose using motavizumab or other 3J20-like mAbs rather than palivizumab in serum antibody competition-binding assays to monitor neutralizing mAbs, as motavizumab competes only with neutralizing mAbs.

As an alternative to full-length RSV F as a vaccine strategy, these data support the concept of using scaffold-based epitopes for immunization against RSV. For example, FFL_001 avoids the potential for non-neutralizing 12I1-like mAb production to compete for binding with neutralizing 14N4-like mAbs, since only the neutralizing epitope is present for an immune response, unlike RSV F where the 12I1 site VII is on an adjacent protomer. Binding to RPM-1 also provides insight into the neutralizing site II epitope, as homologous residues exist in the MPV protein near site VII, yet non-neutralizing RSV-specific antibodies do not bind RPM-1. Thus these scaffold-based immunogens can be used to identify neutralizing mAbs targeting site II, instead of intact RSV F, which also binds non-neutralizing antibodies. As potential vaccines, epitope-scaffold immunogens would not induce site VII mAbs, likely producing only neutralizing mAbs to antigenic site II.

In summary, careful study of the fine specificity of new human antibodies to the RSV F antigenic site II revealed important structural features that inform next-generation vaccine design and testing, and provide new potently neutralizing candidate prophylactic human mAbs.

TABLE 1
NUCLEOTIDE SEQUENCES FOR ANTIBODY VARIABLE REGIONS

| | | SEQ |
|---|---|---|
| Clone | Variable Sequence Region | ID NO: |
| 13A8 | CAGGTGCAGCTGGTGGAGTCTGGGGGAGGCGTGGTCCAGCCTGGGAAGTCCCTG | 15 |
| heavy | AGACTCTCCTGTGCAGCCTCTGGATACATCTTCAGTAGCTATGACATGCACTGG | |
| | GTCCGCCAGGCTCCAGGCAAGGGGCTGGAGTGGGTGGCAGTTATTTCATTTGAC | |
| | GGAACTACTCAACACTATGCAGACTCTGTGAGGGGCCGATTCACCGTCTCCAGA | |
| | GACAATTCCCAGAACACGGTGTTTCTGCAAATGAACAGCCTGAGACCTGAGGAC | |
| | ACGGCTGTGTATTACTGTGTGAAGGAATATGTGATTGTGTCGACTTTCTTTGAC | |
| | TACTGGGGCCAGGGAACCCTGGTCACCGTCTCCTCAG | |
| 13A8 | GACATCGTGATGACCCAGTCTCCATCCTCCCTGTCTGCATCTGTAGGAGACAGA | 16 |
| light | GTCACCATCACTTGCCGGGCAAGTCAGGGCATTAGAAATGCTTTAGGCTGGTAT | |
| | CAGCACAAACCAGGGAAAGCCCCTAAGGTCCTGATCTATGCTGCATCCCGTTTA | |
| | CAAAGTGGGGTCCCATCAAGGTTCAGCGGCAGTGGATCTGGCACAGATTTCACT | |
| | CTCACCATCAGCAGCCTGCAGCCTGAAGATTTTGCAACTTATTACTGTCTTCAA | |
| | GATTTCAATTACCCGTGGACGTTCGGCCACGGGACCAAGGTGGAAATCAAAC | |
| 4E7 | CAGGTGCAGCTGGTGCAGTCTGGGGCTGAGGTGAAGAAGCCTGGGGCCTCACTG | 1 |
| heavy | AAGGTCTCCTACAAGGCCTCTGGATACACCTTCATCGCCTACTATGCGCACTGG | |
| | GTGCGACAGGCCCCTGGACAAGGGCTTGAGTGGATGGGACGGATCAACCCGAAC | |
| | AGTGGTGGCACAAAGTATCACAGAGGTTTCAGGGCAGGGTCACCGTGACCAGGG | |
| | ACACGTCCTTCACCACAGCCTGCCTGGAAATGAACAGGCTAACATCTGACGACA | |
| | CGGCCGTATTTACTTGTGCGAGTAAATATTGCGCTATTGTAGTAGGAGCAGCTG | |
| | CCGTACTCGAGATAGCAACAGCCAAGACCGTCCCCCTCAAGATCGGATGATGGG | |
| | GCCAGGGAACCCTGGTCAGAAGGGATTTGG | |
| 4E7 | CAGTCTGTGGTGACTCAACCACCCTCGACGTCTGGGACCCCCGGGCAGAGGGTC | 2 |
| light | ACCATCTCTTGTTCTGGAAGCAGCGCCAACATCGGAAGAAATGTTGTGAACTGG | |
| | TACCAGCAGGTCCCAGGAACGGCCCCCAAACTCCTCATCTTTGGTAATAGTCAG | |
| | CGGCCCTCAAGGGTCCCTGACCGATTCTCTGGCTCCAAGTCTGGCACCTCAGCC | |
| | TCCCTGGCCATCAGTGGGCTCCAGTCTGAGGATGAGGCTGATTATTATTGTGCA | |
| | ACGTGGGATGACAGCCTGAATGGTCCGGTCTTCGGCGGAGGGACCCAGGTGACC | |
| | GTCCTAG | |
| 10F13 | CAGGTGCAGCTGGTGCAGTCTGGGGGAGGCGTGGTCCAGCCTGGGAGGTCCCTG | 3 |
| heavy | AGACTCTCCTGTGCAGCCTCTGGATTCCCCTTCAGAATCTACTCTATGCACTGG | |
| | GTCCGCCAGGCTCCAGGCAAGGGGCTGGAGTGGGTGGCACTCATCTCATATGAT | |
| | GGAACCAATAAACAGTACGCAGACTCCGTGAACGGCCGATTCACCATCTCCAGA | |
| | GACAATTCCGAGAACACGATGTATTTGCAAATGAACAGTCTGAGACCTGAGGAC | |
| | ACGGCTATCTATTACTGCGCGACAGATATTGTCGAACTGGTGACTGCTACTGAC | |
| | TACTGGGGCCAGGGAACCCTGGTCACCGTCTCCTCAG | |
| 10F13 | AGGCTGTGGTGACTCAGTCTCCAGGCACCCTGTCTTTGTCTCCAGGGGACAGGA | 4 |
| light | GCCACCCTCTCCTGCAGGGCCAGTCAGAGTGTTAGCACCTCCTTAGGCTGGTAC | |
| | CAGCAGAAACCTGGCCAGTCGCCCAGGCTCCTCATCTATGGGACATCCAGAAGG | |
| | GCCACTGGCGTCCCGGACAGGTTCAGTGGCAGTGGATCTGAGACAGACTTCACT | |
| | CTCACCATCAGCAGACTGGAGCCTGAAGATTTTTCAGTGTATTACTGTCAGCAG | |
| | TATGGTAGTTCACCTTACACTTTTGGCCAGGGGACCAGGCTGGAGATCAAAC | |
| 14C16 | CAGGTCCAGCTGGGGGAGTCTGGTCCTGCGCTGGTGAAACCCACACAGACCCTC | 5 |
| heavy | ACACTGACCTGCACGTTCTCTGGGTTCTCACTCAGCACGAGTGAAATGTGTGTG | |
| | AGCTGGATCCGTCAGCCCCCAGGGAAGGCCCTGGAGTGGCTTGCACTCATTGAT | |
| | TGGGATGGTGATAAATTCTTCAGTACATCTCTGCAGTCCAGGCTCACCATCTCC | |
| | AAGAGCCCCTCCAATAACCAGGTGGTCCTTACAATGACCAACATGGACCCTGTG | |
| | GACTCAGGCACCTATTTCTGTGCACGGTCTACTGTTCGCAGGTCGTCCGGCTAC | |
| | TACTACTATGTTTTGGACGTCTGGGGCCAAGGAACCCTGGTCACCGTCTCCTCA | |
| 14C16 | CAGATTGTGATGACTCAGTCTCCATCCTCCCTGTCCGCCTCTGTCGGAGACAGA | 6 |
| light | GTCACCATCAGTTGTCGGGCAAGTCAGAGCATCGGCACCTATGTAAATTGGTAT | |
| | CAACACAAGCCAGGGAAAGCCCCTAAGGTCCTGATCTCTGGTGCCTCCAATTTG | |
| | CACAGTGGGGTCCCATCCAGGTTCAGTGGCAGTGGATCTGGGACAGACTTCACT | |
| | CTCACCATCAGCAGTCTGCAACCTGAAGATTTTGCAACTTACTACTGTCAACAG | |
| | AGTTACAGTCCGCTCACTTTCGGCGGAGGGACCACGGTGGAGATGAAAG | |
| 4B6 | CAGGTGCAGCTGGTGCAGTCTGGGGGAGGCCTGGCACAGCCAGGGCGGTCCCTG | 7 |
| heavy | AGACTCTCCTGTAGAGCTTCTGGGTTCACCTTTGGTGATTTTAATATGAACTGG | |
| | TTCCGCCAGGCTCCAGGGAAGGGGCTGGAGTGGGTAGGATTCATTAGAAGGAAA | |
| | GCTTTTGGTGGGGCAACAGAATACGCCGCGTCGGTGAAAGGCAGACTCACCATC | |
| | TCAAGGGATGATTCCAAGAGCATCGCCTATCTGCAAATGAACAGCCTGAAAACC | |
| | GAGGACACAGCCGTGTATTACTGTACTAGAGAACGGGGATATGTTGGTTCGGGG | |
| | GGGCCCTTCTTTGACTACTGGGGCCAGGGAACCCTGGTCACCGTCTCCTCAG | |
| 4B6 | CAGGCTGTGGTGACTCAGCCGCCCTCAGTGTCTGGGGCCCCAGGGCAGAGGGTC | 8 |
| light | ACCATCTCCTGCACTGGGAGCAGCTCCAACATCGGGGCAGGTTATGATGTACAC | |
| | TGGTACCAGCAACTTCCAGGAACAGCCCCCAAACTCCTCATCTATGGTGACAGC | |
| | AATCGGCCCTCAGGGGTCCCTGACCGATTCTCTGGCTCCAGCTCTGGCACCTCA | |
| | GCCTCCCTGGCCATCACTGGGCTCCAGGCTGAGGATGAGGCTGATTATTACTGC | |
| | CAGTCCTATGACAACAGCCTGAGTGGTTCTGTCTTCGGAACTGGGACCAAGGTC | |
| | ACCGTCCTAG | |
| 9J5 | CAGGTGCAGCTGGTGCAGTCTGGGGCTGAGGTGAAGAAGCCTGGGTCCTCAGTG | 9 |
| heavy | ACGGTCTCCTGCAAGGCTTCTGGAGGCAGCTTCACCAACTATGCTTTCAGCTGG | |
| | GTGCGACAGGCCCCTGGACAAGGGCTTGAGTGGATGGGCGGGATCATCCCTCTC | |
| | CTTAATATGCCAAATTACGCACAGAAGTTTCGGGGCAGAGTCACGATTTCCGCG | |
| | GACCAATCCACCACCACAGCCTACATGGAACTGAGCAGACTGACATCTGAAGAC | |
| | ACGGCCATCTATTTCTGTGCGAGAGGGGGTCAAGTTGGAGATTTTATCGTTCTT | |
| | CGTCACTTTGACTCCTGGGGCCAAGGAACCCTGGTCACCGTCTCCTCAG | |
| 9J5 | CCACCCTCTCCTGCAGGGCCAGTGAGAGTGTTAGCAACTACTTAGCCTGGTATC | 10 |
| light | AGCAGAAACCTGGGCAGACTCCCAGACTCCTCATCTATGGTGCATCCACGAGGG | |
| | CCACTGGTATCCCAGCCAGGTTCAGTGGCAGTGGGTCTGGGTCAGAGTTCACTC | |
| | TCACCATCAGCAGCCTGCAGTCTGAAGATTTTGCGGTTTATTATTGTCAGCAGT | |
| | ATAATGACTGGCCCAGGTTCAGTTTTGGCCAGGGGACCAAGCTGGAGATCAAAC | |
| 12I1 | CAGGTGCAGCTGGTGCAGTCTGGGGGAGGCGTGGTCCAGCCTGGGCAGTCCCTG | 11 |
| heavy | AGACTCTCCTGTGCAGCCTCTGGATTCAGTTTCAGTGACTATCCTATACACTGG | |
| | GTCCGCCAGGCTCCAGGCAAGGGGCTGGAATGGGTGGCAGGAATTTCATATTAT | |
| | GGATCCAATAAATTTTACGCAGACTCCGTGAGGGGCCGCTTCACCATCTCCCGA | |
| | GACACTTCCAAGAACACATTTAATCTGCAAATGAACAGCCTGAAAAGTGAGGAC | |
| | ACGGCTGTGTATTACTGTGCGAGAGATGGCAACCCCCCCCGATTTTTGGAATAC | |
| | TTTGACTACTGGGGCCAGGGAACCCTGGTCACCGTCTCCTCAG | |
| 12I1 | CAGCCTGTGGTGACTCAGCCTCGCTCAGTGTCCGGGTCTCCTGGACAGTCAGTC | 12 |
| light | ACCATCTCCTGCACTGGGAGCAGCAGTGATGTCGGTGGTTATAACTTTGTCTCC | |
| | TGGTACCGACATCACCCAGGCAAGGCCCCCAAACTCCTCATTTATCATGTCACT | |
| | AAGCGGCCCTCAGGGGTCCCTGATCGCTTCTCTGGCTCCAAGTCTGGCAACACG | |
| | GCCTCCCTGACCATCTCTGGGCTCCAGGCTGAGGATGAGGCTGATTATTACTGC | |
| | TGCTCATATGCAGGCAGCTATACTTATGTTCTATTCGGCGGAGGGACCAAGCTG | |
| | ACCGTCCTAG | |
| 14N4 | CAGGTGCAGCTGGTGGAGTCTGGAGGAGGCTTGATCCAGCCTGGGGGGTCCCTG | 13 |
| heavy | AGACTCTCCTGTGCAGTCTCGGGGTTCACCGTCAGTAGCAAGTACATGACCTGG | |
| | GTCCGCCAGGCTCCAGGGAAGGGGCTGGAATGGGTCTCAGTTATTTATGGCGGT | |
| | GGTAGCACATACTACGCAGACTCCGTGGTGGGCCGATTCACCATCTCCAGAGAC | |
| | AATTCCAAGAACACGTTGTATCTTCAAATGAACAGCCTGAGAGCCGAGGACACG | |
| | GCCGTGTATTACTGTGCGAGTCGATTAGGGGTTCGGGCAACTACGGGCGATCTT | |
| | GACTACTGGGGCCAGGGAACCCTGGTCACCGTCTCCTCAG | |
| 14N4 | CAGATTGTGATGACCCAGTCTCCTTCCACCCTGTCTGCATCTGTAGGAGACAGA | 14 |
| light | GTCACCATCACTTGCCGGGCCAGTCAGAGTATTAGTAGCTGGTTGGCCTGGTAT | |
| | CAGCAGAAACCAGGGAAAGCCCCTAAACTCCTGATCTATGATGCCTCCAGTTTG | |
| | GAAAGTGGGGTCCCATCAAGGTTCAGCGGCAGTGGATCTGGGACAGAATTCACT | |
| | CTCACCATCAGCAGCCTGCAGCCTGATGATTTTGCAACTTATTACTGCCAACAG | |
| | TATAATACTTATTCTTGGTGGACGTTCGGCCAAGGGACCAAGGTGGAAATCAAA | |
| | C | |
| 3J20 | GAGGTGCAGCTGGTGGAGTCTGGGGGAGGCTTGGTACAGCCTGGGGGGTCCCTG | 17 |
| heavy | AGACTCTCCTGTGCGGCCTCTGGATTCACCTTTAGCAGTTTTACCATGAACTGG | |
| | GTCCGCCAGGCTCCAGGGAAGGGGCTGCAGTGGGTCTCAACTATTAGTGGTAGT | |
| | GGTGGTCTCACATACTACGCAGACTCCGTGAAGGGCCGGTTCACCATCTCCAGA | |
| | GACAATTCCAAGAACACGCTGTCTCTGCAAATGAACAGCCTGAGAGCCGAGGAC | |
| | ACGGCCGTATATTACTGTGCGAGAGATCTCGAATTTACGGTGACTTCCTACGGG | |
| | GGATACTACTTTGAGTACTGGGGCCAGGGAACCCTGGTCACCGTCTCCTCAG | |
| 3J20 | GAAATTGTGTTGACTCAGTCTCCAGGCACCCTGTCTTTGTCTCCAGGGGAAAGA | 18 |
| light | GCCACCCTCTCCTGCAGGGCCAGTCAGAGTGTTAGCAGCAACTACTTAGCCTGG | |
| | TACCAGCAGAAACCTGGCCAGGCTCCCAGGCTCCTCATCTATGGTGCATCCAGC | |
| | AGGGCCACTGGCATCCCAGACAGGTTCAGTGGCAGTGGGTCTGGGACAGACTTC | |
| | ACTCTCACCATCAGCAGACTGGAGCCTGAAGATTTTGCAGTGTATTACTGTCAG | |
| | CAGTTTGGTAGCTCACCCCGATTCACTTTCGGCCCTGGGACCAAAGTGGATATC | |
| | AAAC | |

TABLE 2
PROTEIN SEQUENCES FOR ANTIBODY VARIABLE REGIONS

| | | SEQ ID |
|---|---|---|
| Clone | Variable Sequence | NO: |
| 13A8 | QVQLVESGGGVVQPGKSLRLSCAASGYIFSSYDMHWVRQAPGKGLEWVA | 34 |
| heavy | VISFDGTTQHYADSVRGRFTVSRDNSQNTVFLQMNSLRPEDTAVYYCVK | |
| | EYVIVSTFFDYWGQGTLVTVSS | |
| 13A8 | DIVMTQSPSSLSASVGDRVTITCRASQGIRNALGWYQHKPGKAPKVLIY | 35 |
| light | AASRLQSGVPSRFSGSGSGTDFILTISSLQPEDFATYYCLQDFNYPWIF | |
| | GHGTKVEIK | |
| 4E7 | QVQLVQSGAEVKKPGASLKVSYKASGYTFIAYYAHWVRQAPGQGLEWMG | 20 |
| heavy | RINPNSGGTKYTQRFQGRVIVIRDTSFTTACLEMNRLTSDDTAVFICAS | |
| | KYCAIVVGAAAVLEIATAKTVPLKIG(N)WGQGTLVRRDL | |
| 4E7 | QSVVTQPPSTSGTPGQRVTISCSGSSANIGRNVVNWYQQVPGTAPKLLI | 21 |
| light | FGNSQRPSRVPDRFSGSKSGTSASLAISGLQSEDEADYYCATWDDSLNG | |
| | PVFGGGTQVTVL | |
| 10F13 | QVQLVQSGGGVVQPGRSLRLSCAASGFPFRIYSMHWVRQAPGKGLEWVA | 22 |
| heavy | LISYDGTNKQYADSVNGRFTISRDNSENTMYLQMNSLRPEDTAIYYCAT | |
| | DIVELVTATDYWGQGTLVTVSS | |
| 10F13 | LSLQAPCLCLQGTGATLSCRASQSVSTSLGWYQQKPGQSPRLLIYGTSR | 23 |
| light | RATGVPDRFSGSGSETDFTLTISRLEPEDFSVYYCQQYGSSPYTFGQGT | |
| | RLEIK | |
| 14C16 | QVQLGESGPALVKPTQTLTLICTFSGFSLSTSEMCVSWIRQPPGKALEW | 24 |
| heavy | LALIDWDGDKFFSTSLQSRLTISKSPSNNQVVLTMTNMDPVDSGTYFCA | |
| | RSTVRRSSGYYYYVLDVWGQGTLVTVSS | |
| 14C16 | QIVMTQSPSSLSASVGDRVTISCRASQSIGTYVNWYQHKPGKAPKVLIS | 25 |
| light | GASNLHSGVPSRFSGSGSGTDFILTISSLQPEDFATYYCQQSYSPLIFG | |
| | GGTTVEMK | |
| 4B6 | QVQLVQSGGGLAQPGRSLRLSCRASGFTFGDFNMNWFRQAPGKGLEWVG | 26 |
| heavy | FIRRKAFGGATEYAASVKGRLTISRDDSKSIAYLQMNSLKTEDTAVYYC | |
| | TRERGYVGSGGPFFDYWGQGTLVTVSS | |
| 4B6 | QAVVTQPPSVSGAPGQRVTISCTGSSSNIGAGYDVHWYQQLPGTAPKLL | 27 |
| light | IYGDSNRPSGVPDRFSGSSSGTSASLAITGLQAEDEADYYCQSYDNSLS | |
| | GSVFGTGTKVTVL | |
| 9J5 | QVQLVQSGAEVKKPGSSVTVSCKASGGSFTNYAFSWVRQAPGQGLEWMG | 28 |
| heavy | GIIPLLNMPNYAQKFRGRVTISADQSITTAYMELSRLTSEDTAIYFCAR | |
| | GGQVGDFIVLRHFDSWGQGTLVTVSS | |
| 9J5 | TLSCRASESVSNYLAWYQQKPGQTPRLLIYGASTRATGIPARFSGSGSG | 29 |
| light | SEFTLTISSLQSEDFAVYYCQQYNDWPRFSFGQGTKLEIK | |
| 12I1 | QVQLVQSGGGVVQPGQSLRLSCAASGFSFSDYPIHWVRQAPGKGLEWVA | 30 |
| heavy | GISYYGSNKFYADSVRGRFTISRDTSKNTFNLQMNSLKSEDTAVYYCAR | |
| | DGNPPRFLEYFDYWGQGTLVTVSS | |
| 12I1 | QPVVTQPRSVSGSPGQSVTISCTGSSSDVGGYNFVSWYRHHPGKAPKLL | 31 |
| light | IYHVTKRPSGVPDRFSGSKSGNTASLTISGLQAEDEADYYCCSYAGSYT | |
| | YVLFGGGTKLTVL | |
| 14N4 | QVQLVESGGGLIQPGGSLRLSCAVSGFTVSSKYMTWVRQAPGKGLEWVS | 32 |
| heavy | VIYGGGSTYYADSVVGRFTISRDNSKNTLYLQMNSLRAEDTAVYYCASR | |
| | LGVRATTGDLDYWGQGTLVTVSS | |
| 14N4 | QIVMTQSPSTLSASVGDRVTITCRASQSISSWLAWYQQKPGKAPKLLIY | 33 |
| light | DASSLESGVPSRFSGSGSGTEFTLTISSLQPDDFATYYCQQYNTYSWWT | |
| | FGQGTKVEIK | |
| 3J20 | EVQLVESGGGLVQPGGSLRLSCAASGFTFSSFTMNWVRQAPGKGLQWVS | 36 |
| heavy | TISGSGGLTYYADSVKGRFTISRDNSKNTLSLQMNSLRAEDTAVYYCAR | |
| | DLEFTVTSYGGYYFEYWGQGTLVTVSS | |
| 3J20 | EIVLTQSPGTLSLSPGERATLSCRASQSVSSNYLAWYQQKPGQAPRLLI | 37 |
| light | YGASSRATGIPDRFSGSGSGTDFTLTISRLEPEDFAVYYCQQFGSSPRF | |
| | TFGPGTKVDIK | |

TABLE 3
CDR HEAVY CHAIN SEQUENCES

| | CDRH1 | CDRH2 | CDRH3 |
|---|---|---|---|
| | (SEQ ID | (SEQ ID | (SEQ ID |
| Antibody | NO:) | NO:) | NO:) |
| 13A8 | GYIFSSYD | ISFDGTTQ | VKEYVIVSTFFDY (61) |
| | (59) | (60) | |
| 4E7 | GYTFIAYY | INPNSGGT | CASKYCAIVVGAAAVLEIATAKTVPLKIG(N)W (40) |
| | (38) | (39) | |
| 10F13 | GFPFRIYS | ISYDGTNK | ATDIVELVTATDY (43) |
| | (41) | (42) | |
| 14C16 | GFSLSTSEMC | IDWDGDK | ARSTVRRSSGYYYYVLDV (46) |
| | (44) | (45) | |
| 4B6 | GFTFGDFN | IRRKAFGGAT | TRERGYVGSGGPFFDY (49) |
| | (47) | (48) | |
| 9J5 | GGSFTNYA | IIPLLNMP | ARGGQVGDFIVLRHFDS (52) |
| | (50) | (51) | |
| 12I1 | GFSFSDYP | ISYYGSNK | ARDGNPPRFLEYFDY (55) |
| | (53) | (54) | |
| 14N4 | GFTVSSKY | IYGGGST | ASRLGVRATTGDLDY (58) |
| | (56) | (57) | |
| 3J20 | GFTFSSFT | ISGSGGLT | ARDLEFTVTSYGGYYFEY (64) |
| | (62) | (63) | |

TABLE 4
CDR LIGHT CHAIN SEQUENCES

| | CDRL1 (SEQ | CDRL2 (SEQ | CDRL3 (SEQ |
|---|---|---|---|
| Antibody | ID NO:) | ID NO:) | ID NO:) |
| 13A8 | QGIRNA | AAS | LQDFNYPWT |
| | (86) | (87) | (88) |
| 4E7 | SANIGRNV | GNS | ATWDDSLNGPV |
| | (65) | (66) | (67) |
| 10F13 | QSVSTS | GTS | QQYGSSPYT |
| | (68) | (69) | (70) |
| 14C16 | QSIGTY | GAS | QQSYSPLT |
| | (71) | (72) | (73) |
| 4B6 | SSNIGAGYD | GDS | QSYDNSLSGSV |
| | (74) | (75) | (76) |
| 9J5 | ESVSNY | GAS | QQYNDWPRFS |
| | (77) | (78) | (79) |
| 12I1 | SSDVGGYNF | HVT | CSYAGSYTYVL |
| | (80) | (81) | (82) |
| 14N4 | QSISSW | DAS | QQYNTYSWWT |
| | (83) | (84) | (85) |
| 3J20 | QSVSSNY | GAS | QQFGSSPRFT |
| | (89) | (90) | (91) |

TABLE 5
Isotype, binding and neutralization features of nine
new RSV F-specific human mAbs or control mAbs

| | | | | | Binding to F protein for indicated | | | |
|---|---|---|---|---|---|---|---|---|
| | | | | | strain (EC₅₀; ng/mL) | | | |
| | Mono- | | | Neutralization | | | | RSV |
| | \|clonal | IgG | Light | (IC₅₀; ng/mL) | | RSV A2 | RSV A2 | 18537 |
| Donor | antibody | subclass | chain | RSV A2 | RSV A2 | DSCav1 | SC-TM | B |
| 2 | 4E7 | 1 | λ | > | 19 | > | 110 | 21 |
| 2 | 10F13 | 1 | κ | > | 17 | 66 | 93 | 21 |
| 1 | 14C16 | 1 | κ | > | 19 | 110 | 95 | 20 |
| 3 | 4B6 | 3 | λ | > | 24 | > | 130 | 24 |
| 1 | 9J5 | 1 | κ | > | 30 | > | 150 | 40 |
| 1 | 12I1 | 1 | λ | > | 26 | > | 250 | 33 |
| 1 | 14N4 | 1 | κ | 695 | 78 | 70 | 57 | 57 |
| 4 | 13A8 | 1 | κ | 55 | 82 | 62 | 52 | 64 |
| 2 | 3J20 | 1 | κ | 377 | 84 | 60 | 48 | 50 |
| Control | motavizumab | 1 | κ | 123 | 30 | 37 | 28 | 35 |
| mAbs | 101F | 1 | κ | 402 | 50 | 62 | 80 | 45 |
| | D25 | 1 | κ | 21 | > | 89 | 72 | > |

EC₅₀ values correspond to the concentration at which half-maximum signal was obtained in enzyme-linked immunosorbent assay, based on optical density at 405 nm.
Neutralization values were determined using a plaque-reduction assay, where the IC₅₀ corresponds to the mAb concentration at which 50% plaque reduction was observed.
> indicates no signal was detected below 100 μg/mL.
DsCav1 and SC-TM represent pre-fusion stabilized RSV F.

TABLE 6
Identification of mAb 14N4-like sequences in a healthy human
donor antibody heavy chain variable gene sequence database

Number of variable region
sequences identified at indicated
percentage match in the HCDR3

| Donor | 85% | 100% |
|---|---|---|
| A | 118 | 99 |
| B | 39 | 33 |
| C | 37 | 36 |
| D | 458 | 398 |
| E | 437 | 387 |
| F | 1 | 1 |
| G | 1 | 1 |
| H | 5 | 5 |
| I | 81 | 68 |
| J | 3 | 3 |
| K | 1 | 1 |
| L | 3 | 3 |
| M | 1 | 1 |
| N | 2 | 2 |

Sequences related to 14N4 are found in many donors. From the inventors' database of 50M+sequences, the inventors identified unique functional sequences (i.e., sequences without stop codons) related to 14N4 using the following clustering protocol: to be considered related, sequences must utilize the same V and J gene as 14N4 (here, IGHV3-53/IGHJ4) and their HCDR3 amino acid sequence must group with 14N4 when clustered at 85% identity using CD-HIT. Of the related sequences, many of them utilized the 14N4 HCDR3 with no amino acid mutations (100% match).

TABLE S1
Data collection and refinement statistics

| | 14N4-Fab | | 14N4-Fab + RSV A2 F | |
|---|---|---|---|---|
| Data collection* | | | | |
| Beamline | Bruker Microstar | | LS-CAT 21-ID-F | |
| Number of crystals | 1 | | 1 | |
| Space group | P 1 2₁ 1 | | P 4₂ 2₁ 2 | |
| Cell dimensions | | | | |
| a, b, c (Å) | 44.5, 75.1, 61.4 | | 235.1, 235.1, 220.1 | |
| α, β, γ (°) | 90, 93.9, 90.0 | | 90, 90, 90 | |
| Resolution (Å) | 28.36-2.00 | (2.07-2.00) | 49.50-4.10 | (4.25-4.10) |
| R_{merge} | 0.118 | (0.496) | 0.296 | (1.19) |
| CC_{1/2} | 0.993 | (0.785) | 0.986 | (0.511) |
| I/σI | 8.8 | (2.2) | 6.0 | (1.9) |
| Completeness (%) | 100 | (100) | 98.1 | (98.7) |
| Redundancy | 4.4 | (3.2) | 8.3 | (8.5) |
| Refinement | | | | |
| Resolution (Å) | 28.36-2.00 | | 49.50-4.10 | |
| No. | 27310 | (2718) | 48002 | (4751) |
| unique reflections | | | | |
| R_{work}/R_{free} | 0.1976/0.2102 | | 0.2562/0.2821 | |
| No. atoms | | | | |
| Protein | 3295 | | 19902 | |
| Water | 411 | | 0 | |
| B-factors | | | | |
| Protein | 19.43 | | 161.69 | |
| Water | 28.03 | | N/A | |
| R.m.s. deviations | | | | |
| Bond lengths (Å) | 0.011 | | 0.009 | |
| Bond angles (°) | 1.35 | | 1.32 | |
| Ramachandran | | | | |
| statistics | | | | |

| | | |
|---|---|---|
| Favored regions (%) | 95.6 | 93.5 |
| Allowed regions | 4.4 | 6.3 |
| (%) | | |
| Outliers (%) | 0 | 0.24 |

*Values in parentheses are for highest-resolution shell.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VII. REFERENCES

The following references, to the extent that they provide exemplary procedural or other details supplementary to those set forth herein, are specifically incorporated herein by reference.
Adams P D, et al. (2010) PHENIX: a comprehensive Python-based system for macromolecular structure solution. Acta Cryst 66(Pt 2):213-21.
“Antibodies: A Laboratory Manual,” Cold Spring Harbor Press, Cold Spring Harbor, N.Y., 1988.
Abbondanzo et al., Am. J Pediatr. Hematol. Oncol., 12(4), 480-489, 1990.
Allred et al., Arch. Surg., 125(1), 107-113, 1990.
Anderson L J, et al. (1985) Antigenic characterization of respiratory syncytial virus strains with monoclonal antibodies. J Infect Dis 151(4):626-633.
Atherton et al., Biol. of Reproduction, 32, 155-171, 1985.
Beeler J A, van Wyke Coelingh K (1989) Neutralization epitopes of the F glycoprotein of respiratory syncytial virus: effect of mutation upon fusion function. J Virol 63(7):2941-2950.
Brown et al., J. Immunol. Meth., 12;130(1), :111-121, 1990.
Campbell, In: Monoclonal Antibody Technology, Laboratory Techniques in Biochemistry and Molecular Biology, Vol. 13, Burden and Von Knippenberg, Eds. pp. 75-83, Amsterdam, Elsevier, 1984.
Capaldi et al., Biochem. Biophys. Res. Comm., 74(2):425-433, 1977.
Correia B E, et al. (2014) Proof of principle for epitope-focused vaccine design. Nature 507:201-206.
Crowe J E, et al. (1998a) Monoclonal antibody-resistant mutants selected with a respiratory syncytial virus-neutralizing human antibody fab fragment (Fab 19) define a unique epitope on the fusion (F) glycoprotein. Virology 252:373-375.
Crowe J E, et al. (1998b) Isolation of a second recombinant human respiratory syncytial virus monoclonal antibody fragment (Fab RSVF2-5) that exhibits therapeutic efficacy in vivo. J Infect Dis 177:1073-1076.
Davidson, E. & Doranz, B. J., Immunology 143, 13-20, 2014.
De Jager et al., Semin. Nucl. Med. 23(2), 165-179, 1993.
Dholakia et al., J. Biol. Chem., 264, 20638-20642, 1989.
Doolittle and Ben-Zeev, Methods Mol. Biol., 109, :215-237, 1999.
Emsley P, Cowtan K (2004) Coot: model-building tools for molecular graphics. Acta Cryst 60(Pt 12 Pt 1):2126-32.
Gefter et al., Somatic Cell Genet., 3:231-236, 1977.
Gilman M S A, et al. (2015) Characterization of a prefusion-specific antibody that recognizes a quaternary, cleavage-dependent epitope on the RSV fusion glycoprotein. PLoS Pathog 11(7):e1005035.
Glenn G M, et al. (2015) A randomized, blinded, controlled, dose-ranging study of a respiratory syncytial virus recombinant fusion (F) nanoparticle vaccine in healthy women of childbearing age. J Infect Dis 213:411-422.
Group TIm-RS (1998) Palivizumab, a humanized respiratory syncytial virus monoclonal antibody, reduces hospitalization from respiratory syncytial virus infection in high-risk infants. Pediatrics 102:531-537.
Gulbis and Galand, Hum. Pathol. 24(12), 1271-1285, 1993.
Hall C B, et al. (2009) The burden of respiratory synctial virus in young children. N Engl J Med 360:588-598.
Kabsch W (2010) Xds. Acta Cryst 66(2):125-132.
Khatoon et al., Ann. of Neurology, 26, 210-219, 1989.
King et al., J. Biol. Chem., 269, 10210-10218, 1989.
Kohler and Milstein, Eur. J. Immunol., 6, 511-519, 1976.
Kohler and Milstein, Nature, 256, 495-497, 1975.
Krarup A, et al. (2015) A highly stable prefusion RSV F vaccine derived from structural analysis of the fusion mechanism. Nat Commun 6:8143.
Kyte and Doolittle, J. Mol. Biol., 157(1):105-132, 1982.
Luo X, et al. (2015) An epitope-specific respiratory syncytial virus vaccine based on an antibody scaffold. Angew Chem Int Ed 54:14531-14534.
McLellan J S, et al. (2013a) Structure of RSV fusion glycoprotein trimer bound to a prefusion-specific neutralizing antibody. Science 340:1113-1117.
McLellan J S, et al. (2013b) Structure-based design of a fusion glycoprotein vaccine for respiratory syncytial virus. Science 342(6158):592-598.
McLellan J S (2015) Neutralizing epitopes on the respiratory syncytial virus fusion glycoprotein. Curr Opin Virol 11:70-75.
Nair H, et al. (2010) Global burden of acute lower respiratory infections due to respiratory syncytial virus in young children: a systematic review and meta-analysis. Lancet 375(9725):1545-1555.
Nakamura et al., In: Enzyme Immunoassays: Heterogeneous and Homogeneous Systems, Chapter 27, 1987.
O'Shannessy et al., J Immun. Meth., 99, 153-161, 1987.
Persic et al., Gene 187:1, 1997
Potter and Haley, Meth. Enzymol., 91, 613-633, 1983.
Raghunandan R, et al. (2014) An insect cell derived respiratory syncytial virus (RSV) F nanoparticle vaccine induces antigenic site II antibodies and protects against RSV challenge in cotton rats by active and passive immunization. Vaccine 32:6485-6492.
Remington's Pharmaceutical Sciences, 15th Ed., 3:624-652, 1990.
Sevy A M, et al. (2013) Epitope mapping of inhibitory antibodies targeting the C2 domain of coagulation factor VIII by hydrogen-deuterium exchange mass spectrometry. J Thromb Haemost 11(12):2128-2136.
Shaikh T R, et al. (2008) SPIDER image processing for single-particle reconstruction of biological macromolecules from electron micrographs. Nat Protoc 3(12):1941-74.
Sheldrick G M (2007) A short history of SHELX. Acta Cryst A64(1):112-122.
Shefali-Patel D, et al. (2012) RSV hospitalisation and healthcare utilisation in moderately prematurely born infants. Eur J Pediatr 171:1055-1061.
Smith G, et al. (2012) Respiratory syncytial virus fusion glycoprotein expressed in insect cells form protein nanoparticles that induce protective immunity in cotton rats. PLoS One 7(11):e50852.
Smith S A, Crowe J E (2015) Use of human hybridoma technology to isolate human monoclonal antibodies. Microbiol Spectr 3:1-12.
Tang et al., J. Biol. Chem., 271:28324-28330, 1996.
Tang G, et al. (2007) EMAN2: An extensible image processing suite for electron microscopy. J Struct Biol 157(1):38-46.
U.S. Pat. No. 3,817,837
U.S. Pat. No. 3,850,752
U.S. Pat. No. 3,939,350
U.S. Pat. No. 3,996,345
U.S. Pat. No. 4,196,265
U.S. Pat. No. 4,275,149
U.S. Pat. No. 4,277,437
U.S. Pat. No. 4,366,241
U.S. Pat. No. 4,472,509
U.S. Pat. No. 4,554,101
U.S. Pat. No. 4,680,338
U.S. Pat. No. 4,816,567
U.S. Pat. No. 4,867,973
U.S. Pat. No. 4,938,948
U.S. Pat. No. 5,021,236
U.S. Pat. No. 5,141,648
U.S. Pat. No. 5,196,066
U.S. Pat. No. 5,563,250
U.S. Pat. No. 5,565,332
U.S. Pat. No. 5,856,456
U.S. Pat. No. 5,880,270
Wawrzynczak & Thorpe, In: Immunoconjugates, Antibody Conuugates In Radioimaging And Therapy Of Cancer, Vogel (Ed.), New York, Oxford University Press, 28, 1987.
Wen X, et al. (2016) A chimeric pneumovirus fusion protein carrying neutralizing epitopes of both MPV and RSV. PLoS One 11:e0155917.
Wu S J, et al. (2007a) Characterization of the epitope for anti-human respiratory syncytial virus F protein monoclonal antibody 101F using synthetic peptides and genetic approaches. J Gen Virol 88(10):2719-2723.
Wu H, et al. (2007b) Development of motavizumab, an ultra-potent antibody for the prevention of respiratory syncytial virus infection in the upper and lower respiratory tract. J Mol Biol 368(3):652-665.
Yu X, McGraw P A, House F S, Crowe J E (2008) An optimized electrofusion-based protocol for generating virus-specific human monoclonal antibodies. J Immunol Methods 336(2):142-151.
Zhao X, Chen F P, Sullender W M (2004) Respiratory syncytial virus escape mutant derived in vitro resists palivizumab prophylaxis in cotton rats. Virology 318:608-612.
Zhao X, Sullender W M (2005) In vivo selection of respiratory syncytial viruses resistant to palivizumab. J Virol 79:3962-3968.
Zhao X, Chen F-P, Megaw A G, Sullender W M (2004) Variable resistance to palivizumab in cotton rats by respiratory syncytial virus mutants. J Infect Dis 190(11):1941-1946.

1. A method of detecting a human respiratory syncytial virus infection in a subject comprising:
(a) contacting a sample from said subject with an antibody or antibody fragment having clone-paired heavy and light chain CDR sequences from Tables 3 and 4, respectively; and
(b) detecting human respiratory syncytial virus in said sample by binding of said antibody or antibody fragment to a Human respiratory syncytial virus antigen in said sample.

2-12. (canceled)

13. A method of treating a subject infected with human respiratory syncytial virus, or reducing the likelihood of infection of a subject at risk of contracting human respiratory syncytial virus, comprising delivering to said subject an antibody or antibody fragment having clone-paired heavy and light chain CDR sequences from Tables 3 and 4, respectively.

14. The method of claim 13, the antibody or antibody fragment is encoded by clone-paired light and heavy chain variable sequences as set forth in Table 1.

15. The method of claim 13, the antibody or antibody fragment is encoded by clone-paired light and heavy chain variable sequences having 95% identify to as set forth in Table 1.

16. The method of claim 13, wherein said antibody or antibody fragment is encoded by light and heavy chain variable sequences having 70%, 80%, or 90% identity to clone-paired sequences from Table 1.

17. The method of claim 13, wherein said antibody or antibody fragment comprises light and heavy chain variable sequences according to clone-paired sequences from Table 2.

18. The method of claim 13, wherein said antibody or antibody fragment comprises light and heavy chain variable sequences having 70%, 80% or 90% identity to clone-paired sequences from Table 2.

19. The method of claim 13, encoded by light and heavy chain variable sequences having 95% identity to clone-paired sequences from Table 2.

20. The method of claim 13, wherein the antibody fragment is a recombinant ScFv (single chain fragment variable) antibody, Fab fragment, F(ab′)₂ fragment, or Fv fragment, a chimeric antibody and/or is an IgG.

21. The method of claim 13, wherein said antibody or antibody fragment recognizes an epitope on RSV F protein in antigenic site II.

22. The method of claim 21, wherein said antibody or antibody fragment escapes competition with non-neutralizing site II antibodies.

23. The method of claim 13, wherein said antibody or antibody fragment is administered prior to infection.

24. The method of claim 13, wherein said antibody or antibody fragment is administered after infection.

25. The method of claim 13, wherein delivering comprises antibody or antibody fragment administration, or genetic delivery with an RNA or DNA sequence or vector encoding the antibody or antibody fragment.

26-35. (canceled)

36. A hybridoma or engineered cell encoding an antibody or antibody fragment wherein the antibody or antibody fragment is characterized by clone-paired heavy and light chain CDR sequences from Tables 3 and 4, respectively.

37-46. (canceled)

47. A vaccine formulation comprising one or more antibodies or antibody fragments characterized by clone-paired heavy and light chain CDR sequences from Tables 3 and 4, respectively.

48-52. (canceled)

53. The vaccine formulation of claim 47, wherein at least one of said antibody fragments is a recombinant ScFv (single chain fragment variable) antibody, Fab fragment, F(ab′)₂ fragment, or Fv fragment, or wherein at least one of said antibodies is a chimeric antibody, is bispecific antibody, and/or is an IgG.

54. (canceled)

55. The vaccine formulation of claim 47, wherein said antibody or antibody fragment recognizes an epitope on RSV F protein in antigenic site II, and optionally escapes competition with non-neutralizing site II antibodies.

56. The vaccine formulation of claim 47, wherein at least one of said antibodies or antibody fragments further comprises a cell penetrating peptide and/or is an intrabody.

57. A method of identifying an anti-human respiratory syncytial virus (hRSV) protein F site II-specific neutralizing antibody comprising:
(a) contacting a candidate antibody with hRSV protein F in the presence of a known site II-specific neutralizing antibody or antigen binding fragment thereof;
(b) assessing binding of said candidate antibody to hRSV protein F; and
(c) identifying said candidate antibody as a protein F site II-specific neutralizing antibody when said known site II-specific neutralizing antibody or antigen binding fragment thereof blocks binding of said candidate antibody to hRSV protein F.

58-66. (canceled)
